(19) United States
(12) Patent Application Publication
(10) Pub. No.: US 2024/0382258 A1
(43) Pub. Date: Nov. 21, 2024

(54) Inflatable Balloon and Vessel Diameter Correlation System for an Intravascular Lithotripsy Device

(71) Applicant: BOLT MEDICAL, INC., Carlsbad, CA (US)

(72) Inventors: Eric SCHULTHEIS, San Clemente, CA (US); Christopher A. COOK, Laguna Niguel, CA (US)

(21) Appl. No.: 18/430,050

(22) Filed: Feb. 1, 2024

Related U.S. Application Data
(60) Provisional application No. 63/514,481, filed on Jul. 19, 2023.
(60) Provisional application No. 63/443,487, filed on Feb. 6, 2023.

(51) Int. Cl. A61B 18/26; A61B 18/00; A61B 18/04; A61B 18/20

(52) U.S. Cl. CPC A61B 18/26; A61B 18/042; A61B 2018/0022; A61B 2018/00404; A61B 2018/00642; A61B 2018/00666; A61B 2018/2035

(57) ABSTRACT

A catheter system (100) for treating a treatment site (106) within or adjacent to a vessel wall (108A) of a blood vessel (108) includes a balloon (104), a first assembly illumination source (366) and a contact detector assembly (142). The balloon (104) is positionable substantially adjacent to the vessel wall (108A) at the treatment site (106). The balloon (104) has a balloon wall (130) that defines a balloon interior (146). The first assembly illumination source (366) generates a first assembly illumination beam (366B) that moves in a first direction (121F) into the balloon interior (146). The contact detector assembly (142) is configured to optically analyze a first returning energy beam (366R) from the balloon interior (146) that moves in a second direction (121S) that is opposite the first direction (121F), the contact detector assembly (142) being configured to analyze the first returning energy beam (366R) to determine a contact condition between the balloon wall (130) and the vessel wall (108A).

### RELATED APPLICATIONS

This application claims priority from U.S. Provisional Patent application Ser. No. 63/443,487, filed on Feb. 6, 2023, and entitled “VESSEL WALL CONTACT DETECTOR ASSEMBLY FOR INTRAVASCULAR LITHOTRIPSY DEVICE”, and from U.S. Provisional Patent Application Ser. No. 63/514,481, filed on Jul. 19, 2023, and entitled “INFLATABLE BALLOON AND VESSEL DIAMETER CORRELATION SYSTEM FOR AN INTRAVASCULAR LITHOTRIPSY DEVICE”. To the extent permissible, the contents of U.S. Provisional Patent Application Ser. No. 63/443,487, and U.S. Provisional Patent Application Ser. No. 63/514,481 are incorporated in their entirety herein by reference.

### BACKGROUND

Vascular lesions within vessels in the body can be associated with an increased risk for major adverse events, such as myocardial infarction, embolism, deep vein thrombosis, stroke, and the like. Severe vascular lesions can be difficult to treat and achieve patency for a physician in a clinical setting.

Vascular lesions may be treated using interventions such as drug therapy, balloon angioplasty, atherectomy, stent placement, and vascular graft bypass, to name a few. Such interventions may not always be ideal or may require subsequent treatment to address the lesion.

Angioplasty balloons are typically semi-compliant inflatable balloons (also sometimes referred to herein as “balloons”), which means that the outer diameter of the balloon changes slightly as pressure is applied to the balloon. An “Instructions For Use” (IFU) manual is typically provided for each catheter, and a balloon compliance chart is typically provided within the IFU manual. The balloon compliance chart plots the outer diameter of the balloon versus the internal balloon pressure so that the physician can know the outer diameter of the balloon as pressure is applied. Since the IFU manual is not sterile, it is challenging for the physician to have easy access to it during the procedure since the physician must remain sterile. For this reason, the balloon compliance chart and/or the IFU manual is often not used during procedures.

The outer diameter of the balloon is also an important metric for the physician to understand since it is necessary that the outer diameter of the balloon be sized appropriately relative to the inner diameter of the vessel in which the balloon is being used. The correct sizing of the outer diameter of the balloon relative to the inner diameter of the vessel allows for optimal energy delivery because the wall of the balloon must be in contact with the vessel wall to most effectively deliver the desired lithotripsy therapy. If the balloon is not in contact with the vessel wall, then the mechanics of the energy delivery are significantly reduced. It is further appreciated that overstretching the vessel, which can occur when the outer diameter of the balloon exceeds the inner diameter of the vessel (measured at a point in time prior to the vessel being overstretched by the balloon), can be a safety concern that could result in serious vessel trauma, such as vessel tear or dissections.

In many implementations, intravascular lithotripsy (IVL) is performed in the peripheral vessel prior to subsequent use of a drug-coated balloon catheter (DCB). The IVL therapy breaks apart the calcium and allows for the drug on the DCB catheter to wick into the cracked areas and treat deep into the vessel wall, including delivering drug to the adventitia wall (outermost wall). If the outer diameter of the DCB catheter is smaller than the inner diameter of the vessel wall, then the delivery of the drug on the DCB catheter is reduced and may result in ineffective treatment.

Several DCB clinical trial studies in below-the-knee (BTK) blood vessels have been unsuccessful, and it is speculated that inappropriate balloon sizing may have caused the poor result. In these studies, angiograms (contrast injections into the vessel while viewing under fluoroscopy) were used to assess and measure vessel diameter. Separate studies have shown that this method is an inaccurate way to measure the vessel and could result in measurements off by as much as one millimeter (mm). Considering that some of the peripheral vessels have diameters that range from two mm to eight mm, the inaccuracy can be significant and result in balloon outer diameter to vessel inner diameter mismatch. This is particularly true for the BTK blood vessels that have vessel diameters of between two mm and four mm.

As a result of these studies, many physicians have been measuring the vessel diameter with intravascular ultrasound (IVUS) catheters and/or optical coherence topography (OCT) catheters to get an accurate vessel morphology and size prior to DCB use. These catheters are expensive, adding thousands of dollars to the procedural cost. Also, the preparation, use, and resulting interpretation of the catheters is time consuming, adding significant time to the procedure. It also requires staff that are trained to use the diagnostic catheter and console.

### SUMMARY

The present invention is directed toward a catheter system for treating a treatment site within or adjacent to a vessel wall of a blood vessel. In various embodiments, the catheter system includes a balloon, a first assembly illumination source and a contact detector assembly. The balloon is positionable substantially adjacent to the vessel wall at the treatment site. The balloon has a balloon wall that defines a balloon interior. The first assembly illumination source generates a first assembly illumination beam that moves in a first direction into the balloon interior. The contact detector assembly is configured to optically analyze a first returning energy beam from the balloon interior that moves in a second direction that is opposite the first direction, the contact detector assembly being configured to analyze the first returning energy beam to determine a contact condition between the balloon wall and the vessel wall.

In many embodiments, the balloon is configured to receive and retain a catheter fluid within the balloon interior.

In certain embodiments, the catheter system further includes a pressure sensor assembly including a pressure sensor that is configured to sense an internal balloon pressure of the catheter fluid within the balloon interior.

In some embodiments, the pressure sensor is in fluid communication with the catheter fluid retained within the balloon interior.

In many embodiments, the catheter system further includes a balloon compliance chart that plots an outer diameter of the balloon versus the internal balloon pressure, and a balloon diameter determination system that determines the outer diameter of the balloon based on the sensed internal balloon pressure and the balloon compliance chart.

In certain embodiments, the catheter system further includes a vessel diameter determination system that is configured to determine an inner diameter of the blood vessel.

In some embodiments, the contact detector assembly is configured to analyze the first returning energy beam to determine when good contact exists between the balloon wall and the vessel wall.

In certain embodiments, when the contact detector assembly determines that good contact exists between the balloon wall and the vessel wall, the vessel diameter determination system determines that the inner diameter of the blood vessel is equal to the outer diameter of the balloon.

In some embodiments, the treatment site has a site length; and the vessel diameter determination system is configured to determine the inner diameter of the blood vessel at multiple locations along the site length of the treatment site.

In many embodiments, the catheter system further includes a system controller including one or more processors, and a diameter correlation system that is at least partially integrated within the system controller.

In some embodiments, the diameter correlation system is configured to utilize data from the balloon diameter determination system regarding the outer diameter of the balloon, and data from the vessel diameter determination system regarding the inner diameter of the blood vessel at the multiple locations along the site length of the treatment site, for purposes of ensuring a proper correlation of the outer diameter of the balloon and the inner diameter of the blood vessel during use of the catheter system in a therapeutic procedure.

In some embodiments, the catheter system further includes a beam guide. The first assembly illumination beam moves in the first direction through the beam guide from a guide proximal end to a guide distal end that is positioned within the balloon interior. The first illumination beam includes first light energy The first returning energy beam moves in the second direction through the beam guide from the guide distal end to the guide proximal end. The first returning energy beam includes second light energy from at least a portion of the first light energy being reflected from the vessel wall.

In many embodiments, the catheter system further includes a second assembly illumination source that generates a second assembly illumination beam that moves in the first direction into the balloon interior.

In some embodiments, the contact detector assembly is configured to optically analyze a second returning energy beam from the balloon interior that moves in the second direction that is opposite the first direction, the contact detector assembly being configured to analyze the first returning energy beam and the second returning energy beam to determine the contact condition between the balloon wall and the vessel wall.

In certain embodiments, the second assembly illumination beam moves in the first direction through the beam guide from the guide proximal end to the guide distal end that is positioned within the balloon interior, the second assembly illumination beam including first light energy; and the second returning energy beam moves in the second direction through the beam guide from the guide distal end to the guide proximal end, the second returning energy beam including second light energy from at least a portion of the first light energy from the second assembly illumination beam being reflected from blood that is positioned between the balloon wall and the vessel wall.

In various embodiments, the first assembly illumination beam is at a first wavelength, and the second assembly illumination beam is at a second wavelength that is different than the first wavelength.

In some embodiments, the catheter system further includes an energy source that generates a source beam that is directed into the balloon interior. In certain embodiments, the source beam is directed through the beam guide from a guide proximal end to a guide distal end that is positioned within the balloon interior. In some embodiments, the balloon is configured to receive and retain a catheter fluid within the balloon interior; and the source beam being directed into the balloon interior induces generation of a plasma in the catheter fluid within the balloon interior.

In other embodiments, the catheter system further includes an energy source that generates a source beam that is directed into the balloon interior; and an energy guide that is separate from the beam guide. In some embodiments, the source beam is directed through the energy guide from a guide proximal end to a guide distal end that is positioned within the balloon interior. In certain embodiments, the balloon interior is configured to receive and retain a catheter fluid within the balloon interior, and the source beam being directed into the balloon interior induces generation of a plasma in the catheter fluid within the balloon interior.

In several embodiments, the contact detector assembly includes a beamsplitter and a photodetector, the beamsplitter being configured to receive returning energy that has moved through the beam guide in the second direction from the guide distal end to the guide proximal end, and direct at least a portion of the returning energy to the photodetector.

In some embodiments, the photodetector generates a signal based at least in part on the portion of the returning energy that is directed to the photodetector, the signal being used by control electronics to determine the contact condition between the balloon wall and the vessel wall.

The present invention is further directed toward a method for treating a treatment site within or adjacent to a vessel wall of a blood vessel, including the steps of positioning a balloon substantially adjacent to the vessel wall at the treatment site, the balloon having a balloon wall that defines a balloon interior; generating a first assembly illumination beam with a first assembly illumination source; moving the first assembly illumination beam in a first direction into the balloon interior; moving a first returning energy beam from the balloon interior in a second direction that is opposite the first direction; and optically analyzing the first returning energy beam from the balloon interior with a contact detector assembly to determine a contact condition between the balloon wall and the vessel wall.

The present invention is also directed toward a catheter system for treating a treatment site within or adjacent to a vessel wall of a blood vessel, the treatment site having a site length, the catheter system including (A) a balloon that is positionable substantially adjacent to the vessel wall at the treatment site, the balloon having a balloon wall that defines a balloon interior, the balloon being configured to receive and retain a catheter fluid within the balloon interior; (B) a pressure sensor assembly including a pressure sensor that is configured to sense an internal balloon pressure of the catheter fluid within the balloon interior, the pressure sensor being in fluid communication with the catheter fluid retained within the balloon interior; (C) a balloon compliance chart that plots an outer diameter of the balloon versus the internal balloon pressure; (D) a balloon diameter determination system that determines the outer diameter of the balloon based on the sensed internal balloon pressure and the balloon compliance chart, (E) a beam guide including a guide proximal end and a guide distal end, the guide distal end being positioned within the balloon interior; (F) a first assembly illumination source that generates a first assembly illumination beam that moves through the beam guide in a first direction from the guide proximal end to the guide distal end, the first assembly illumination beam including first light energy that is at a first wavelength, the first assembly illumination beam being directed from the guide distal end toward the vessel wall; (G) a second assembly illumination source that generates a second assembly illumination beam that moves through the beam guide in the first direction from the guide proximal end to the guide distal end, the second assembly illumination beam including first light energy that is at a second wavelength that is different than the first wavelength, the second assembly illumination beam being directed from the guide distal end toward the vessel wall; (H) a contact detector assembly that is configured to optically analyze (i) a first returning energy beam from the balloon interior that moves through the beam guide in a second direction from the guide distal end to the guide proximal end, the first returning energy beam including second light energy from at least a portion of the first light energy being reflected from the vessel wall, and (ii) a second returning energy beam from the balloon interior that moves through the beam guide in the second direction from the guide distal end to the guide proximal end, the second returning energy beam including second light energy from at least a portion of the first light energy from the second assembly illumination beam being reflected from blood that is positioned between the balloon wall and the vessel wall, the contact detector assembly being configured to analyze the first returning energy beam and the second returning energy beam to determine a contact condition between the balloon wall and the vessel wall; (I) a vessel diameter determination system that is configured to determine an inner diameter of the blood vessel; wherein when the contact detector assembly determines that good contact exists between the balloon wall and the vessel wall, the vessel diameter determination system determines that the inner diameter of the blood vessel is equal to the outer diameter of the balloon, the vessel diameter determination system being configured to determine the inner diameter of the blood vessel at multiple locations along the site length of the treatment site; (J) a system controller including one or more processors; and (K) a diameter correlation system that is at least partially integrated within the system controller, the diameter correlation system being configured to utilize data from the balloon diameter determination system regarding the outer diameter of the balloon, and data from the vessel diameter determination system regarding the inner diameter of the blood vessel at the multiple locations along the site length of the treatment site, for purposes of ensuring a proper correlation of the outer diameter of the balloon and the inner diameter of the blood vessel during use of the catheter system in a therapeutic procedure.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic cross-sectional view illustration of an embodiment of a catheter system in accordance with various embodiments herein, the catheter system including a pressure sensor assembly and a vessel wall contact detector assembly that are usable as part of an inflatable balloon and vessel diameter correlation system (also sometimes referred to herein as “diameter correlation system”) having features of the present invention;

FIG. 2 is a flowchart illustrating a first operational mode for an embodiment of the catheter system including an embodiment of the vessel wall contact detector assembly;

FIG. 3A is a simplified schematic view illustration of a portion of the embodiment of the catheter system including the embodiment of the vessel wall contact detector assembly usable within the first operational mode as described in relation to FIG. 2;

FIG. 3B is a simplified schematic view illustration of the portion of the embodiment of the catheter system including the embodiment of the vessel wall contact detector assembly illustrated in FIG. 3A, with a step within the first operational mode being illustrated;

FIG. 3C is a simplified schematic view illustration of the portion of the embodiment of the catheter system including the embodiment of the vessel wall contact detector assembly illustrated in FIG. 3A, with another step within the first operational mode being illustrated;

FIG. 3D is a simplified schematic view illustration of the portion of the embodiment of the catheter system including the embodiment of the vessel wall contact detector assembly illustrated in FIG. 3A, with still another step within the first operational mode being illustrated;

FIG. 3E is a simplified schematic view illustration of the portion of the embodiment of the catheter system including the embodiment of the vessel wall contact detector assembly illustrated in FIG. 3A, with yet another step within the first operational mode being illustrated;

FIG. 3F is a simplified schematic view illustration of the portion of the embodiment of the catheter system including the embodiment of the vessel wall contact detector assembly illustrated in FIG. 3A, with still yet another step within the first operational mode being illustrated;

FIG. 4 is a flowchart illustrating a second operational mode for the embodiment of the catheter system including the embodiment of the vessel wall contact detector assembly of FIG. 3A;

FIG. 5A is a simplified schematic view illustration of the portion of the embodiment of the catheter system including the embodiment of the vessel wall contact detector assembly of FIG. 3A, with a step within the second operational mode being illustrated;

FIG. 5B is a simplified schematic view illustration of the portion of the embodiment of the catheter system including the embodiment of the vessel wall contact detector assembly of FIG. 3A, with another step within the second operational mode being illustrated;

FIG. 5C is a simplified schematic view illustration of the portion of the embodiment of the catheter system including the embodiment of the vessel wall contact detector assembly illustrated in FIG. 3A, with still another step within the second operational mode being illustrated;

FIG. 6 is a flowchart illustrating an operational mode for another embodiment of the catheter system including another embodiment of the vessel wall contact detector assembly;

FIG. 7A is a simplified schematic view illustration of a portion of the embodiment of the catheter system including the embodiment of the vessel wall contact detector assembly usable within the operational mode as described in relation to FIG. 6;

FIG. 7B is a simplified schematic view illustration of the portion of the embodiment of the catheter system including the embodiment of the vessel wall contact detector assembly illustrated in FIG. 7A, with a step within the operational mode being illustrated;

FIG. 7C is a simplified schematic view illustration of the embodiment of the catheter system including the embodiment of the vessel wall contact detector assembly illustrated in FIG. 7A, with another step within the operational mode being illustrated;

FIG. 7D is a simplified schematic view illustration of the portion of the embodiment of the catheter system including the embodiment of the vessel wall contact detector assembly illustrated in FIG. 7A, with still another step within the operational mode being illustrated;

FIG. 8 is a simplified schematic view illustration of still another embodiment of the catheter system including still another embodiment of the vessel wall contact detector assembly;

FIG. 9 is a simplified schematic view illustration of an embodiment of the diameter correlation system having features of the present invention, the diameter correlation system incorporating input from and/or output for the pressure sensor assembly, a balloon compliance chart, a balloon diameter determination system, the vessel wall contact detector assembly, a vessel diameter determination system, and a vessel diameter mapping system;

FIG. 10 is a flowchart illustrating a first (testing and/or mapping) mode of operation for the diameter correlation system of FIG. 9;

FIG. 11A is an image illustrating a method for ensuring proper positioning of a catheter, a balloon and/or emitters of the catheter system relative to a treatment site;

FIG. 11B is an image illustrating a full site length of the treatment site, with markings at specified periodic locations along the site length showing an inner diameter of a blood vessel at such locations as part of the vessel diameter mapping system; and

FIG. 12 is a flowchart illustrating a second (therapeutic use) mode of operation for the diameter correlation system of FIG. 9.

While embodiments of the present invention are susceptible to various modifications and alternative forms, specifics thereof have been shown by way of example and drawings, and are described in detail herein. It is understood, however, that the scope herein is not limited to the particular embodiments described. On the contrary, the intention is to cover modifications, equivalents, and alternatives falling within the spirit and scope herein.

### DESCRIPTION

Treatment of vascular lesions at treatment sites within a body of a patient can reduce major adverse events or death in affected subjects. As referred to herein, a major adverse event is one that can occur anywhere within the body due to the presence of a vascular lesion. Major adverse events can include, but are not limited to, major adverse cardiac events, major adverse events in the peripheral or central vasculature, major adverse events in the brain, major adverse events in the musculature, or major adverse events in any of the internal organs.

In various embodiments, the catheter systems and related methods disclosed herein can include a catheter configured to advance to a vascular lesion, such as a calcified vascular lesion or a fibrous vascular lesion, at a treatment site located within a body of a patient. As used herein, the “treatment site” can be located at or near a vessel wall of a blood vessel of the patient. For example, in some implementations, the “treatment site” can be found in below-the-knee (BTK) blood vessels, which can have a longer site length (sometimes up to 30 centimeters or more), and which can have somewhat narrower inner diameters that can vary along the site length. In other implementations, the “treatment site” can be located at or near a vessel wall of any other blood vessel of the patient. Additionally, or in the alternative, the “treatment site” can be at or near a heart valve of the patient. Further, or in the alternative, the “treatment site” can be at another suitable location within the body of the patient.

Moreover, as used herein, the terms “intravascular lesion”, “vascular lesion” and “treatment site” are used interchangeably unless otherwise noted. The intravascular lesions and/or the vascular lesions are sometimes referred to herein simply as “lesions”.

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it is appreciated that such a development effort might be complex and time-consuming, but would nevertheless be a routine undertaking of engineering for those of ordinary skill in the art having the benefit of this disclosure.

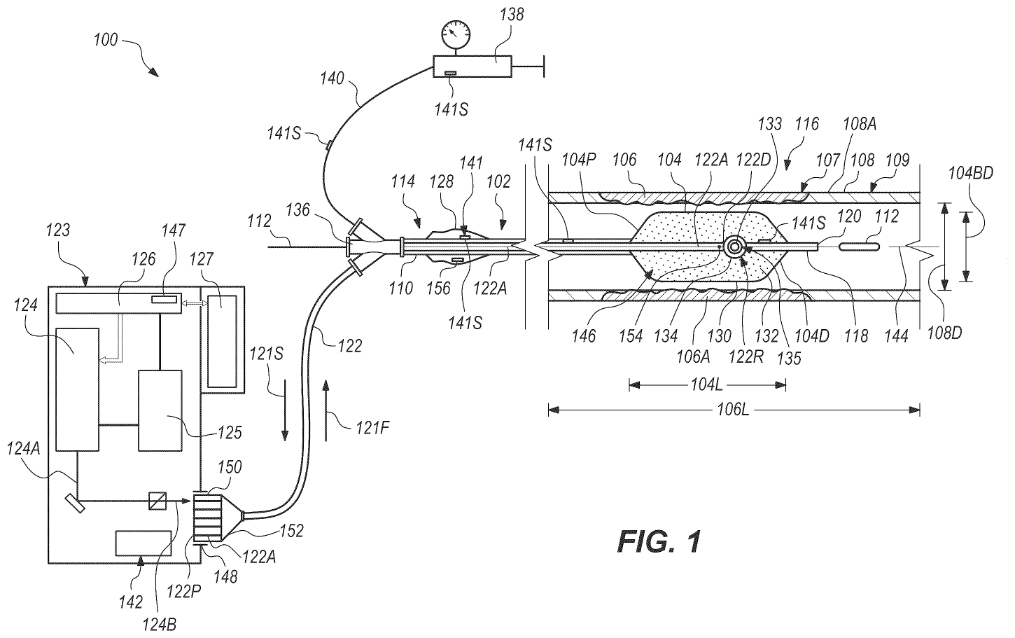

The catheter systems disclosed herein can include many different forms. Referring now to FIG. 1, a schematic cross-sectional view illustration is shown of a catheter system 100 in accordance with various embodiments. In some implementations, the catheter system 100 is suitable for imparting pressure waves to induce fractures in one or more treatment sites within or adjacent to a vessel wall of a blood vessel, or on or adjacent to a heart valve, within a body of a patient. In certain implementations, the catheter system 100 is suitable for delivering drugs to the vessel wall in the form of anti-inflammatory agents, anti-neoplastic agents, anti-angiogenic agents, and the like.

In the embodiment illustrated in FIG. 1, the catheter system 100 can include (i) a catheter 102 including one or more of an inflatable balloon 104 (sometimes referred to herein as a “balloon”), a catheter shaft 110, a guidewire 112, an energy guide bundle 122 including one or more energy guides 122A, a source manifold 136, a fluid pump 138, a handle assembly 128, and a pressure sensor assembly 141 including one or more pressure sensors 141S; and (ii) a system console 123 including one or more of an energy source 124, a power source 125, a system controller 126, a graphic user interface 127 (a “GUI”), and a vessel wall contact detector assembly 142 (also sometimes referred to as a “contact detector assembly” or a “contact detector”). Alternatively, the catheter system 100, the catheter 102 and/or the system console 123 can include more components or fewer components than those specifically illustrated and described in relation to FIG. 1.

The catheter 102 is configured to move to a treatment site 106 at any suitable location within a body 107 of a patient 109. In some implementations, the treatment site 106 can be within or adjacent to a vessel wall 108A of a blood vessel 108 within a body 107 of a patient 109. Alternatively, in other implementations, the catheter 102 can be used at a treatment site 106 within or adjacent to a heart valve within the body 107 of the patient 109.

As shown, the treatment site 106 can have a site length 106L that can be any suitable or desired length. For example, the site length 106L can be up to 30 centimeters or more, such as when the treatment site 106 is in below-the-knee (BTK) blood vessels. Alternatively, the treatment site 106 can have another suitable site length 106L.

The treatment site 106 can include one or more vascular lesions 106A such as calcified vascular lesions, for example. Additionally, or in the alternative, the treatment site 106 can include vascular lesions 106A such as fibrous vascular lesions. It is appreciated that the vascular lesions 106A found at the treatment site 106 will help define the site length 106L of the treatment site 106.

In certain embodiments, the balloon 104 can be coupled to the catheter shaft 110. The balloon 104 can include a balloon proximal end 104P and a balloon distal end 104D. The catheter shaft 110 can extend from a proximal portion 114 of the catheter system 100 to a distal portion 116 of the catheter system 100. The catheter shaft 110 can include a longitudinal axis 144. The catheter 102 and/or the catheter shaft 110 can also include a guidewire lumen 118 which is configured to move over the guidewire 112. As utilized herein, the guidewire lumen 118 defines a conduit through which the guidewire 112 extends. The catheter shaft 110 can further include an inflation lumen (not shown) and/or various other lumens for various other purposes. In some embodiments, the catheter 102 can have a distal end opening 120 and can accommodate and be tracked over the guidewire 112 as the catheter 102 is moved and positioned at or near the treatment site 106. In some embodiments, the balloon proximal end 104P can be coupled to the catheter shaft 110, and the balloon distal end 104D can be coupled to the guidewire lumen 118.

The balloon 104 includes a balloon wall 130 that defines a balloon interior 146. The balloon 104 can be selectively inflated with a catheter fluid 132 to expand from a deflated state suitable for advancing the catheter 102 through a patient's vasculature, to an inflated state (as shown in FIG. 1) suitable for anchoring the catheter 102 in position relative to the treatment site 106. Stated in another manner, when the balloon 104 is in the inflated state, the balloon wall 130 of the balloon 104 is configured to be positioned substantially directly adjacent to and/or in contact with the treatment site 106. It is appreciated that although FIG. 1 illustrates the balloon wall 130 of the balloon 104 being shown spaced apart from the treatment site 106 of the blood vessel 108 when in the inflated state, this is done merely for ease of illustration. It is recognized that the balloon wall 130 of the balloon 104 will typically, as desired, be substantially directly adjacent to and/or abutting or in contact with the treatment site 106 when the balloon 104 is in the inflated state.

In many embodiments, the present invention discloses a system and method for accurately estimating balloon diameter and vessel diameter, and thus ensuring the desired correlation between an outer diameter 104BD of the balloon 104 and an inner diameter 108D of the blood vessel 108, in order to provide enhanced treatment efficacy during use of an intravascular lithotripsy device and/or a drug-coated balloon catheter in a manner that is both time-efficient and cost-effective. Stated in another manner, the present invention discloses a system and method for ensuring that the balloon wall 130 of the balloon 104 is substantially directly adjacent to and/or abutting or in contact with the vessel wall 108A of the blood vessel 108 at the treatment site 106 when the balloon 104 is in the inflated state. It is appreciated that the systems and methods disclosed herein are equally applicable whether the catheter system 100 is being utilized as an intravascular lithotripsy device for purposes of breaking apart the vascular lesions 106A at the treatment site 106 and/or whether the catheter system 100 is being utilized as a drug-coated balloon catheter for purposes of delivering drug therapy to the treatment site 106 in order to treat deep into the vessel wall 108A of the blood vessel 108.

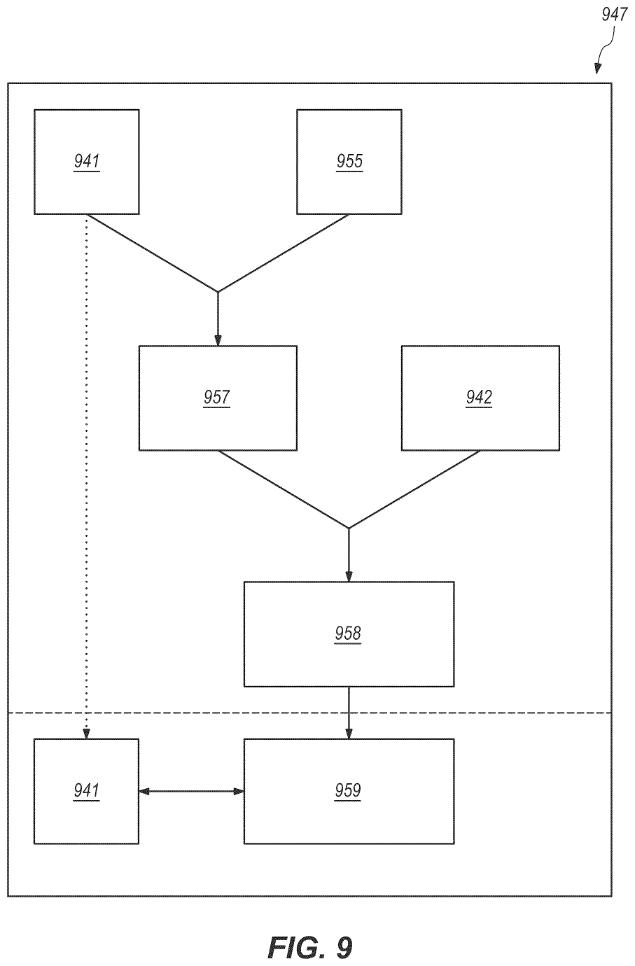

As an overview, in many embodiments, the system controller 126 can utilize data and/or information from the pressure sensor assembly 141, such as from the one or more pressure sensors 141S, and the contact detector assembly 142 as part of a balloon and vessel diameter correlation system 147 (illustrated as a box positioned within the system controller 126, and sometimes referred to herein as a “diameter correlation system”) for purposes of ensuring a proper correlation and/or matching of the outer diameter 104BD of the balloon 104 and the inner diameter 108D of the blood vessel 108 for most effective lithotripsy therapy and/or drug delivery at the treatment site 106. In particular, the system controller 126 can utilize (i) data and/or information from the pressure sensor assembly 141 regarding an internal balloon pressure, such as from the one or more pressure sensors 141S, in conjunction with a balloon compliance chart 955 (illustrated in FIG. 9) that plots the outer diameter 104BD of the balloon 104 versus the internal balloon pressure, to determine the outer diameter 104BD of the balloon 104 as part of a balloon diameter determination system 957 (illustrated in FIG. 9); (ii) data and/or information from the balloon diameter determination system 957 with regard to the outer diameter 140BD of the balloon 104, in conjunction with data and/or information from the contact detector assembly 142 with regard to whether or not good contact exists between the balloon wall 130 and the vessel wall 108A, to determine the inner diameter 108D of the blood vessel 108 as part of a vessel diameter determination system 958 (illustrated in FIG. 9); (iii) data and/or information from the vessel diameter determination system 958 at a plurality of locations along the site length 106L of the treatment site 106 as part of a vessel diameter mapping system 959 (illustrated in FIG. 9); and (iv) data and/or information from the vessel diameter mapping system 959, in conjunction with data and/or information from the balloon diameter determination system 957, as part of the diameter correlation system 147 for purposes of increasing the likelihood of obtaining a proper correlation (or matching) of the outer diameter 104BD of the balloon 104 and the inner diameter 108D of the blood vessel 108 for most effective lithotripsy therapy and/or drug delivery at the treatment site 106.

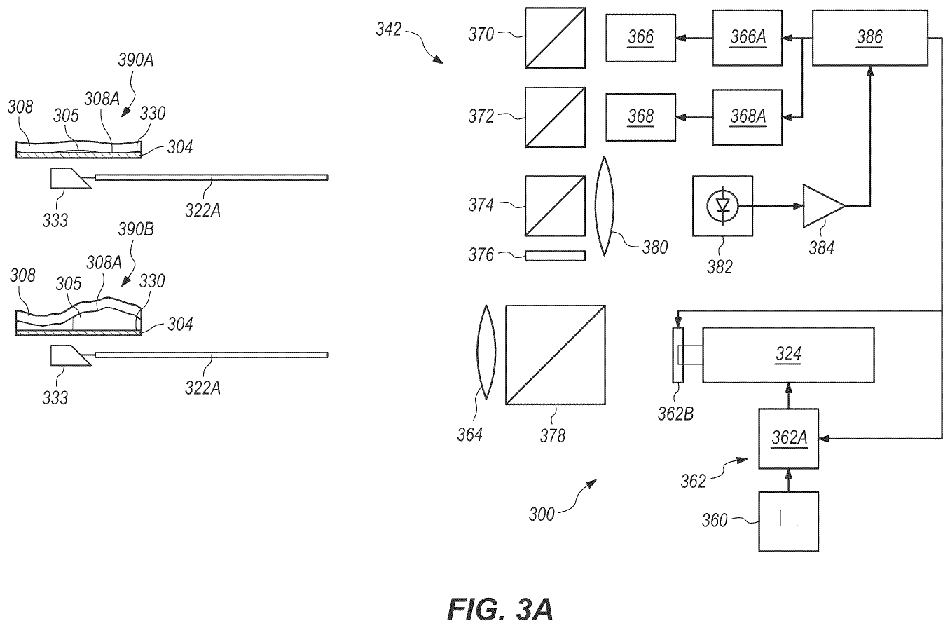

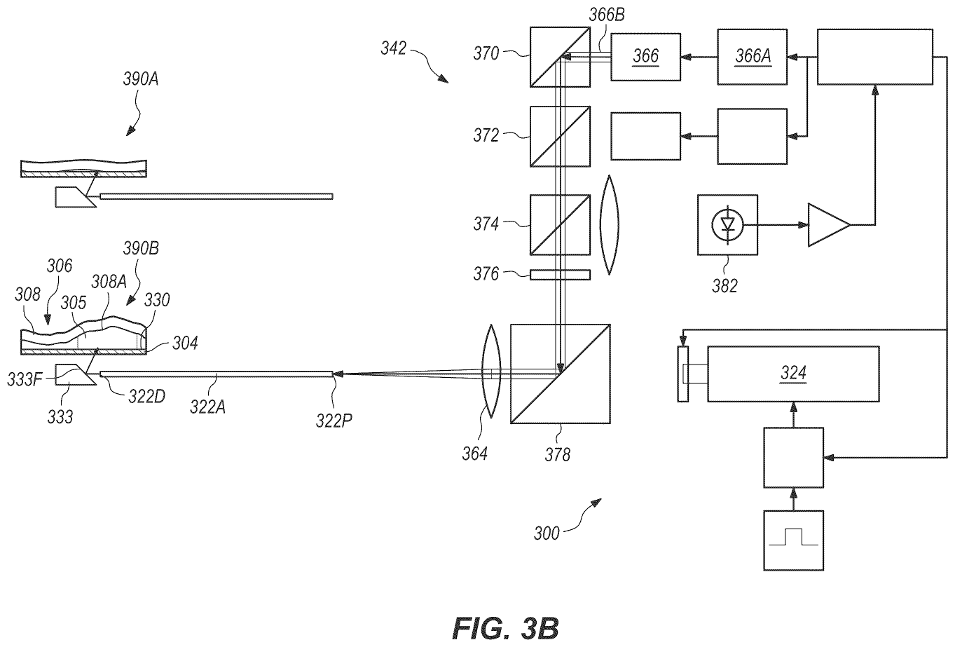

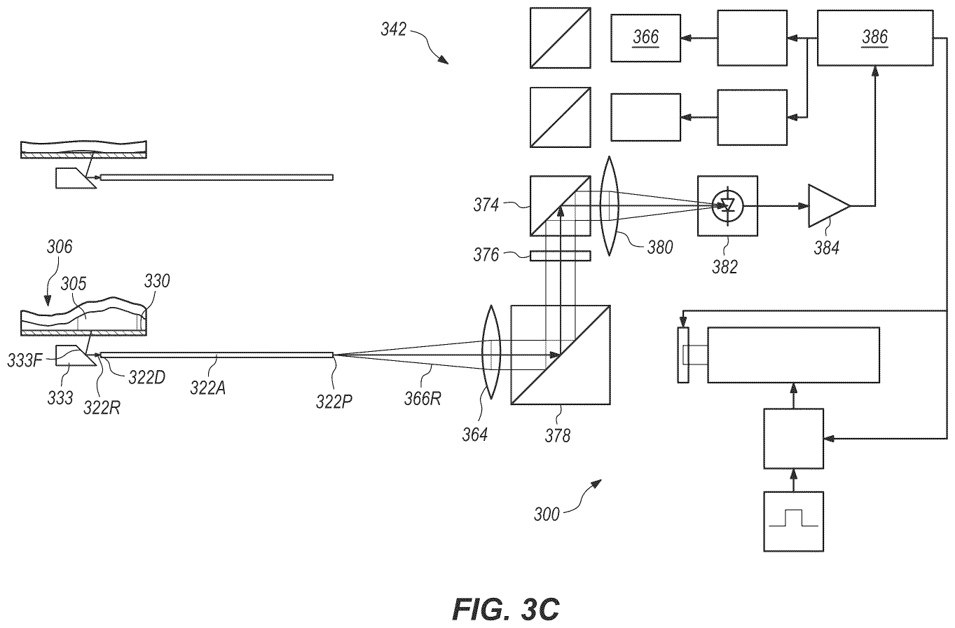

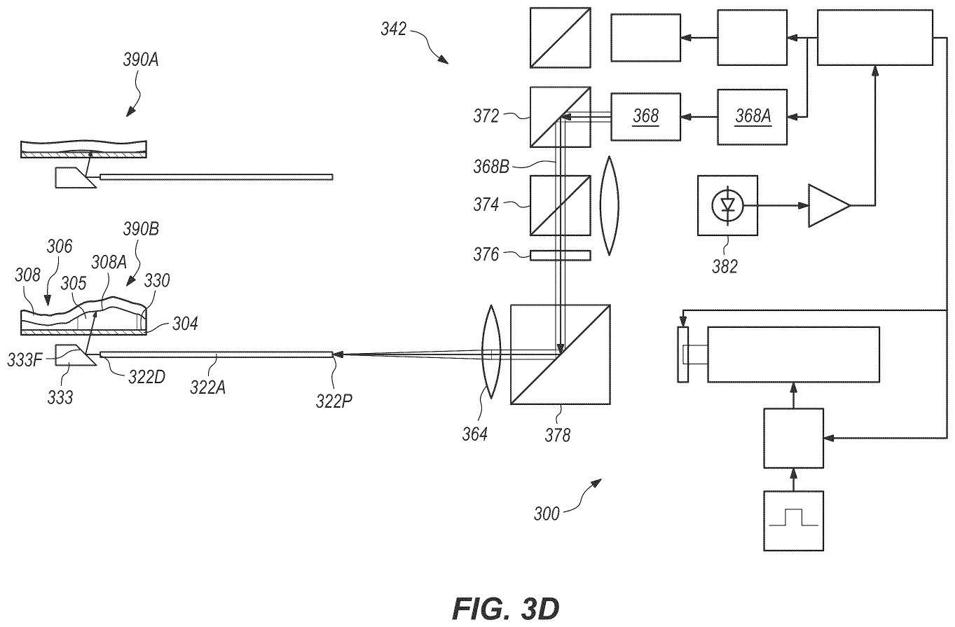

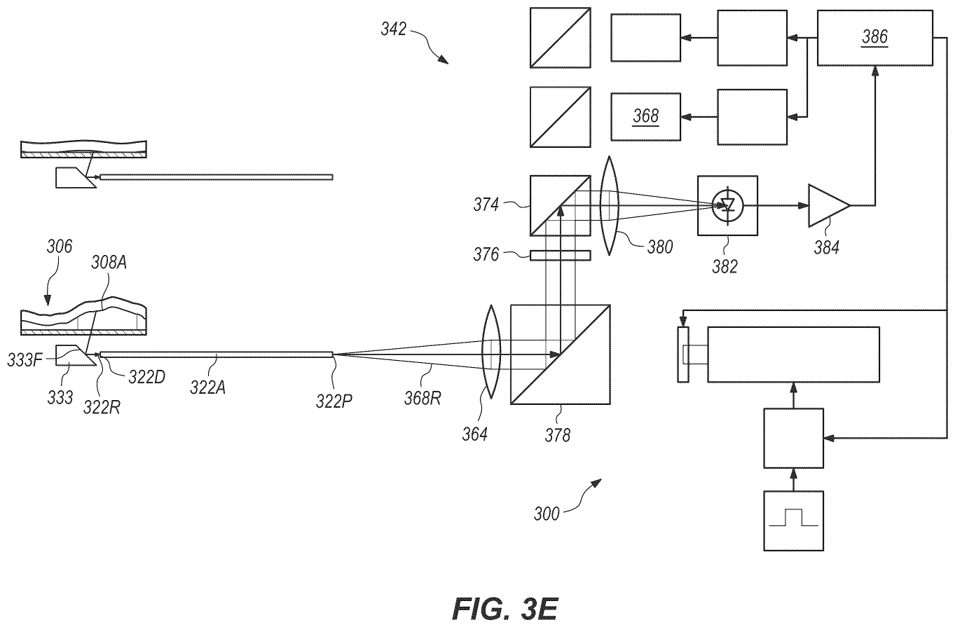

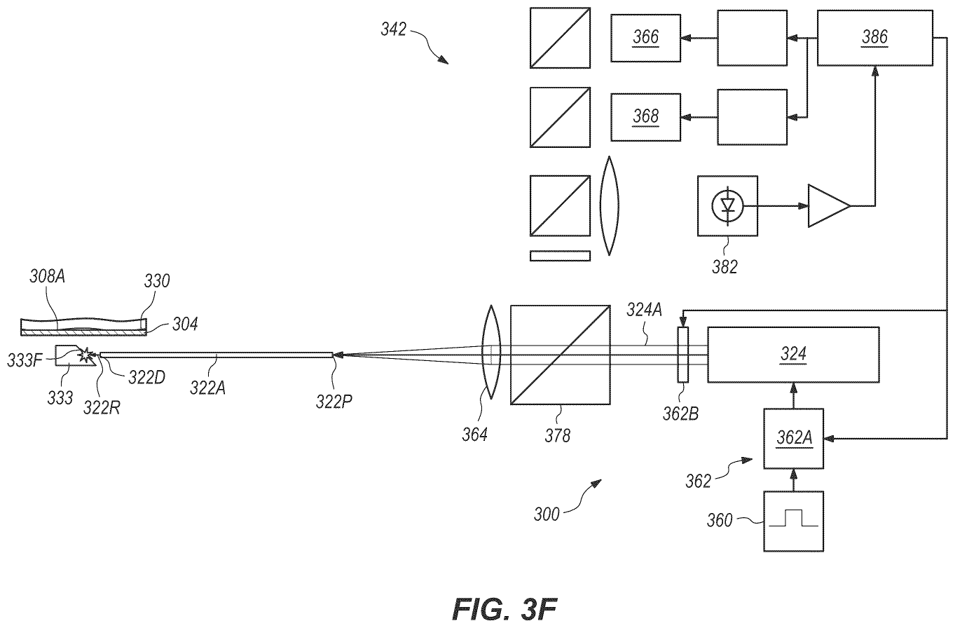

It is recognized that when the balloon 104 is positioned adjacent to the treatment site 106 of the blood vessel 108, it is desired that very little or no blood 305 (illustrated, for example, in FIG. 3A), or other fluids, be positioned between the balloon wall 130 and the vessel wall 108A. Unfortunately, if the balloon 104 is not of an appropriate size, such as if the balloon 104 is undersized for the blood vessel 108 under treatment, or if the balloon 104 is not appropriately inflated, a volume of blood 305 can be present between the balloon wall 130 and the vessel wall 108A that can have a negative impact on the energy transmission and treatment efficacy of the catheter system 100. Thus, the contact detector assembly 142 of the present invention is utilized to detect whether the balloon wall 130 is or is not in optimal contact with the vessel wall 108A, so that energy transmission and treatment efficacy can be optimized.

The balloon 104 suitable for use in the catheter system 100 includes those that can be passed through the vasculature of a patient 109 when in the deflated state. In some embodiments, the balloon 104 is made from silicone. In other embodiments, the balloon 104 can be made from polydimethylsiloxane (PDMS), polyurethane, polymers such as PEBAX™ material, nylon, or any other suitable material.

The balloon 104 can have any suitable diameter (in the inflated state). In various embodiments, the balloon 104 can have a diameter (in the inflated state) ranging from less than one millimeter (mm) up to 25 mm. In some embodiments, the balloon 104 can have a diameter (in the inflated state) ranging from at least 1.5 mm up to 14 mm. In certain embodiments, the balloon 104 can have a diameter (in the inflated state) ranging from at least two mm up to five mm.

In some embodiments, the balloon 104 can have a balloon length 104L ranging from at least three mm to 300 mm. More particularly, in some embodiments, the balloon 104 can have a balloon length 104L ranging from at least eight mm to 200 mm. It is appreciated that a balloon 104 having a relatively longer length can be positioned adjacent to larger treatment sites 106, and, thus, may be usable for imparting pressure waves onto and inducing fractures in larger vascular lesions 106A or multiple vascular lesions 106A at precise locations within the treatment site 106. It is further appreciated that a longer balloon 104 can also be positioned adjacent to multiple treatment sites 106 at any one given time. It is also appreciated that when the site length 106L of the treatment site 106 is very long, such as up to 30 cm or more in below-the-knee (BTK) blood vessels, the balloon 104 may need to be moved to multiple different locations during a procedure in order to effectively treat the entire site length 106L of the treatment site 106.

The balloon 104 can be inflated to inflation pressures of between approximately one atmosphere (atm) and 70 atm. In some embodiments, the balloon 104 can be inflated to inflation pressures of from at least 20 atm to 60 atm. In other embodiments, the balloon 104 can be inflated to inflation pressures of from at least six atm to 20 atm. In still other embodiments, the balloon 104 can be inflated to inflation pressures of from at least three atm to 20 atm. In yet other embodiments, the balloon 104 can be inflated to inflation pressures of from at least two atm to ten atm.

As described herein, it is appreciated that the inflation pressure of the balloon 104 (also referred to as the “internal balloon pressure”) impacts the outer diameter 104BD of the balloon 104, and thus impacts the proper correlation between the outer diameter 104BD of the balloon 104 and the inner diameter 108D of the blood vessel 108 as part of the diameter correlation system 147.

The balloon 104 can have various shapes, including, but not to be limited to, a conical shape, a square shape, a rectangular shape, a spherical shape, a conical/square shape, a conical/spherical shape, an extended spherical shape, an oval shape, a tapered shape, a bone shape, a stepped diameter shape, an offset shape, or a conical offset shape. In some embodiments, the balloon 104 can include a drug eluting coating or a drug eluting stent structure. The drug eluting coating or drug eluting stent can include one or more therapeutic agents including anti-inflammatory agents, anti-neoplastic agents, anti-angiogenic agents, and the like.

The catheter fluid 132 used to inflate the balloon 104 can be a liquid or a gas. Some examples of the catheter fluid 132 suitable for use can include, but are not limited to one or more of water, saline, contrast medium, fluorocarbons, perfluorocarbons, gases, such as carbon dioxide, or any other suitable catheter fluid 132. In some embodiments, the catheter fluid 132 can be used as a base inflation fluid. In some embodiments, the catheter fluid 132 can include a mixture of saline to contrast medium in a volume ratio of approximately 50:50. In other embodiments, the catheter fluid 132 can include a mixture of saline to contrast medium in a volume ratio of approximately 25:75. In still other embodiments, the catheter fluid 132 can include a mixture of saline to contrast medium in a volume ratio of approximately 75:25. However, it is understood that any suitable ratio of saline to contrast medium can be used. The catheter fluid 132 can be tailored on the basis of composition, viscosity, and the like so that the rate of travel of the pressure waves are appropriately manipulated. In certain embodiments, the catheter fluid 132 suitable for use herein is biocompatible. A volume of catheter fluid 132 can be tailored by the chosen energy source 124 and the type of catheter fluid 132 used.

In some embodiments, the contrast agents used in the contrast media can include, but are not to be limited to, iodine-based contrast agents, such as ionic or non-ionic iodine-based contrast agents. Some non-limiting examples of ionic iodine-based contrast agents include diatrizoate, metrizoate, iothalamate, and ioxaglate. Some non-limiting examples of non-ionic iodine-based contrast agents include iopamidol, iohexol, ioxilan, iopromide, iodixanol, and ioversol. In other embodiments, non-iodine-based contrast agents can be used. Suitable non-iodine containing contrast agents can include gadolinium (III)-based contrast agents. Suitable fluorocarbon and perfluorocarbon agents can include, but are not to be limited to, agents such as perfluorocarbon dodecafluoropentane (DDFP, C5F12).

The catheter fluids 132 can include those that include absorptive agents that can selectively absorb light in the ultraviolet region (e.g., at least ten nanometers (nm) to 400 nm), the visible region (e.g., at least 400 nm to 780 nm), or the near-infrared region (e.g., at least 780 nm to 2.5 μm) of the electromagnetic spectrum. Suitable absorptive agents can include those with absorption maxima along the spectrum from at least ten nm to 2.5 μm. Alternatively, the catheter fluids 132 can include those that include absorptive agents that can selectively absorb light in the mid-infrared region (e.g., at least 2.5 μm to 15 μm), or the far-infrared region (e.g., at least 15 μm to one mm) of the electromagnetic spectrum. In various embodiments, the absorptive agent can be those that have an absorption maximum matched with the emission maximum of the laser used in the catheter system 100. By way of non-limiting examples, various lasers usable in the catheter system 100 can include neodymium:yttrium-aluminum-garnet (Nd:YAG—emission maximum=1064 nm) lasers, holmium:YAG (Ho:YAG—emission maximum=2.1 μm) lasers, or erbium:YAG (Er:YAG—emission maximum=2.94 μm) lasers. In some embodiments, the absorptive agents can be water soluble. In other embodiments, the absorptive agents are not water soluble. In some embodiments, the absorptive agents used in the catheter fluids 132 can be tailored to match the peak emission of the energy source 124. Various energy sources 124 having emission wavelengths of at least ten nanometers to one millimeter are discussed elsewhere herein.

The catheter shaft 110 of the catheter 102 can be coupled to the one or more energy guides 122A of the energy guide bundle 122 that are in optical communication with the energy source 124. The energy guide(s) 122A can be disposed along the catheter shaft 110 and within the balloon 104. Each of the energy guides 122A can have a guide distal end 122D that is at any suitable longitudinal position relative to the balloon length 104L of the balloon 104 and/or relative to a length of the guidewire lumen 118.

In some embodiments, each energy guide 122A can be an optical fiber and the energy source 124 can be a laser. The energy source 124 can be in optical communication with the energy guides 122A at the proximal portion 114 of the catheter system 100. More particularly, the energy source 124 can selectively, simultaneously, sequentially and/or alternatively be in optical communication with each of the energy guides 122A in any desired combination, sequence and/or pattern.

In some embodiments, the catheter shaft 110 can be coupled to multiple energy guides 122A such as a first energy guide, a second energy guide, a third energy guide, etc., which can be disposed at any suitable positions about and/or relative to the guidewire lumen 118 and/or the catheter shaft 110. For example, in certain non-exclusive embodiments, two energy guides 122A can be spaced apart from one another by approximately 180 degrees about the circumference of the guidewire lumen 118 and/or the catheter shaft 110; three energy guides 122A can be spaced apart from one another by approximately 120 degrees about the circumference of the guidewire lumen 118 and/or the catheter shaft 110; four energy guides 122A can be spaced apart from one another by approximately 90 degrees about the circumference of the guidewire lumen 118 and/or the catheter shaft 110; five energy guides 122A can be spaced apart from one another by approximately 72 degrees about the circumference of the guidewire lumen 118 and/or the catheter shaft 110; six energy guides 122A can be spaced apart from one another by approximately 60 degrees about the circumference of the guidewire lumen 118 and/or the catheter shaft 110; eight energy guides 122A can be spaced apart from one another by approximately 45 degrees about the circumference of the guidewire lumen 118 and/or the catheter shaft 110; or ten energy guides 122A can be spaced apart from one another by approximately 36 degrees about the circumference of the guidewire lumen 118 and/or the catheter shaft 110. Still alternatively, multiple energy guides 122A need not be uniformly spaced apart from one another about the circumference of the guidewire lumen 118 and/or the catheter shaft 110. More particularly, it is further appreciated that the energy guides 122A can be disposed uniformly or non-uniformly about the guidewire lumen 118 and/or the catheter shaft 110 to achieve the desired effect in the desired locations.

In certain embodiments, the guidewire lumen 118 can have a grooved outer surface, with the grooves extending in a generally longitudinal direction along the guidewire lumen 118. In such embodiments, each of the energy guides 122A can be positioned, received, and retained within an individual groove formed along and/or into the outer surface of the guidewire lumen 118. Alternatively, the guidewire lumen 118 can be formed without a grooved outer surface, and the position of the energy guides 122A relative to the guidewire lumen 118 can be maintained in another suitable manner.

The catheter system 100, the catheter 102 and/or the energy guide bundle 122 can include any number of energy guides 122A in optical communication with the energy source 124 at the proximal portion 114, and with the catheter fluid 132 within the balloon interior 146 of the balloon 104 at the distal portion 116. For example, in some embodiments, the catheter system 100, the catheter 102 and/or the energy guide bundle 122 can include from one energy guide 122A to greater than 30 energy guides 122A. The guide distal end 122D of each of the energy guides 122A can be at any suitable or desired longitudinal position within the balloon interior 146 relative to the balloon length 104L of the balloon 104. Alternatively, in other embodiments, the catheter system 100, the catheter 102 and/or the energy guide bundle 122 can include greater than 30 energy guides 122A.

The energy guides 122A can have any suitable design that is useful and appropriate for purposes of enabling the generation of plasma and/or pressure waves in the catheter fluid 132 within the balloon interior 146. Thus, the general description of the energy guides 122A as light guides is not intended to be limiting in any manner, except for as set forth in the claims appended hereto. More particularly, although the catheter systems 100 are often described with the energy source 124 as a light source and the one or more energy guides 122A as light guides, the catheter system 100 can alternatively include any suitable energy source 124 and energy guides 122A for purposes of enabling the generation of the desired plasma in the catheter fluid 132 within the balloon interior 146. For example, in one non-exclusive alternative embodiment, the energy source 124 can be configured to provide high voltage pulses, and each energy guide 122A can include an electrode pair including spaced apart electrodes that extend into the balloon interior 146. In such embodiment, each pulse of high voltage is applied to the electrodes and forms an electrical arc across the electrodes, which, in turn, generates the plasma and forms the pressure waves in the catheter fluid 132 that are utilized to provide the fracture force onto the vascular lesions 106A at the treatment site 106. Still alternatively, the energy source 124 and/or the energy guides 122A can have another suitable design and/or configuration, be it electrical, acoustic, pneumatic, other mechanical, etc.

As illustrated, the catheter system 100 can include one or more emitters 135 that are configured to generate plasma and/or pressure waves in the catheter fluid 132 within the balloon interior 146. Each of the emitters 135 includes a guide distal end 122D of one of the energy guides 122A, which is positioned within the balloon interior 146, and a corresponding plasma generating structure 133 (also referred to herein as a “plasma generator”) that is positioned near, but typically spaced apart from, the guide distal end 122D. Energy from the energy source 124 is directed toward and received by the energy guide 122A, is guided through the energy guide 122A, and is then emitted from the guide distal end 122D of the energy guide 122A. The energy emitted from the guide distal end 122D is directed toward and impinges on and energizes the corresponding plasma generator 133 for purposes of generating the plasma in the catheter fluid 132 within the balloon interior 146.

In certain embodiments, the emitters 135 can be formed from and/or can include a radiopaque material that is easily visible when used with fluoroscopy during an intravascular lithotripsy procedure. The visibility of the emitters 135 through use of the radiopaque material enables the user or operator to more precisely position the emitters 135 as desired substantially adjacent to the vascular lesions 106A, and/or to selectively activate only those emitters 135 that are positioned most proximate to the vascular lesions 106A in order to more effectively disrupt the vascular lesions 106A. Alternatively, the emitters 135 can be formed from other suitable materials that can be made visible to the user or operator during an intravascular lithotripsy procedure.

By positioning the emitters 135 more precisely substantially adjacent to the vascular lesions 106A at the treatment site 106, and by activating only certain emitters 135 based on proximity to the vascular lesions 106A at the treatment site 106, the user or operator can operate the catheter system 100 more effectively and efficiently. Thus, the user and operator can realize savings in money and resources.

In certain embodiments, the energy guides 122A can include an optical fiber or flexible light pipe. The energy guides 122A can be thin and flexible and can allow light signals to be sent with very little loss of strength. The energy guides 122A can include a core surrounded by a cladding about its circumference. In some embodiments, the core can be a cylindrical core or a partially cylindrical core. The core and cladding of the energy guides 122A can be formed from one or more materials, including but not limited to one or more types of glass, silica, or one or more polymers. The energy guides 122A may also include a protective coating, such as a polymer. It is appreciated that the index of refraction of the core will be greater than the index of refraction of the cladding.

Each energy guide 122A can guide first energy along its length from a guide proximal end 122P toward the guide distal end 122D, with the guide distal end 122D having at least one optical window (not shown) that is positioned within the balloon interior 146. In some implementations, the first energy can originate from the energy source 124 before being guided along the energy guide 122A from the guide proximal end 122P to the guide distal end 122D and into the balloon interior 146. First energy that originates from the energy source 124 can be utilized for performing therapeutic treatment procedures with the catheter system 100 to treat and/or disrupt the vascular lesions 106A at the treatment site 106. In other implementations, the first energy can originate from one or more assembly illumination sources 366, 368 (or simply “illumination sources”, illustrated, for example, in FIG. 3A) that can be included as part of the contact detector assembly 142. As described in greater detail herein below, first energy that originates from the illumination sources 366, 368 can be used to illuminate regions at or near the guide distal end 122D and/or at or near the treatment site 106, which can then generate second energy at or near the treatment site 106 that can be subsequently utilized by the contact detector assembly 142 to determine a status of contact between the balloon wall 130 and the vessel wall 108A.

In various embodiments, the guide distal end 122D of each energy guide 122A can include and/or incorporate a distal light receiver 122R that is a structural entity that is positioned at or near the guide distal end 122D of the energy guide 122A and is configured to capture and/or receive the second energy and to enable the second energy to be moved back into and through the energy guide 122A from the guide distal end 122D to the guide proximal end 122P. Stated another way, the first energy can move in a first direction 121F along the energy guide 122A that is generally from the guide proximal end 122P toward the guide distal end 122D of the energy guide 122A. The second energy, which in certain situations can comprise at least a portion of the first energy, can move in a second direction 121S along the energy guide 122A that is substantially opposite the first direction 121F, such as from at or near the guide distal end 122D toward the guide proximal end 122P of the energy guide 122A. Moreover, as described in greater detail herein below, the second energy emitted from the guide proximal end 122P after being moved back through the energy guide 122A (in the second direction 121S) can be separated and then optically detected, interrogated and/or analyzed through use of the contact detector assembly 142 in order to determine whether or not the balloon wall 130 of the balloon 104 is in optimal contact with the vessel wall 108A of the blood vessel 108 at the treatment site 106.

The energy guides 122A can assume many configurations about and/or relative to the catheter shaft 110 of the catheter 102. In some embodiments, the energy guides 122A can run parallel to the longitudinal axis 144 of the catheter shaft 110. In some embodiments, the energy guides 122A can be physically coupled to the catheter shaft 110. In other embodiments, the energy guides 122A can be disposed along a length of an outer diameter of the catheter shaft 110. In yet other embodiments, the energy guides 122A can be disposed within one or more energy guide lumens within the catheter shaft 110.

The energy guides 122A can also be disposed at any suitable positions about the circumference of the guidewire lumen 118 and/or the catheter shaft 110, and the guide distal end 122D of each of the energy guides 122A can be disposed at any suitable longitudinal position relative to the balloon length 104L of the balloon 104 and/or relative to the length of the guidewire lumen 118 to more effectively and more precisely impart pressure waves for purposes of disrupting the vascular lesions 106A at the treatment site 106.

In certain embodiments, the energy guides 122A can include one or more photoacoustic transducers 154, where each photoacoustic transducer 154 can be in optical communication with the energy guide 122A within which it is disposed. In some embodiments, the photoacoustic transducers 154 can be in optical communication with the guide distal end 122D of the energy guide 122A. In such embodiments, the photoacoustic transducers 154 can have a shape that corresponds with and/or conforms to the guide distal end 122D of the energy guide 122A.

The photoacoustic transducer 154 is configured to convert first energy into an acoustic wave at or near the guide distal end 122D of the energy guide 122A. The direction of the acoustic wave can be tailored by changing an angle of the guide distal end 122D of the energy guide 122A.

In certain embodiments, the photoacoustic transducers 154 disposed at the guide distal end 122D of the energy guide 122A can assume the same shape as the guide distal end 122D of the energy guide 122A. For example, in certain non-exclusive embodiments, the photoacoustic transducer 154 and/or the guide distal end 122D can have a conical shape, a convex shape, a concave shape, a bulbous shape, a square shape, a stepped shape, a half-circle shape, an ovoid shape, and the like. The energy guide 122A can further include additional photoacoustic transducers 154 disposed along one or more side surfaces of the length of the energy guide 122A.

In some embodiments, the energy guides 122A can further include one or more diverting structures or “diverters” (not shown in FIG. 1), such as within the energy guide 122A and/or near the guide distal end 122D of the energy guide 122A, that are configured to direct energy from the energy guide 122A toward a side surface which can be located at or near the guide distal end 122D of the energy guide 122A, before the energy is directed toward the balloon wall 130. A diverting structure can include any structure of the system that diverts energy from the energy guide 122A away from its axial path toward a side surface of the energy guide 122A. The energy guides 122A can each include one or more optical windows disposed along the longitudinal or circumferential surfaces of each energy guide 122A and in optical communication with a diverting structure. Stated in another manner, the diverting structures can have any suitable structural configuration that is configured to direct energy in the energy guide 122A toward a side surface that is at or near the guide distal end 122D, where the side surface is in optical communication with an optical window. The optical windows can include a portion of the energy guide 122A that allows energy to exit the energy guide 122A from within the energy guide 122A, such as a portion of the energy guide 122A lacking a cladding material on or about the energy guide 122A.

Examples of the diverting structures suitable for use include a reflecting element, a refracting element, and a fiber diffuser. The diverting structures suitable for focusing first energy away from the tip of the energy guides 122A can include, but are not to be limited to, those having a convex surface, a gradient-index (GRIN) lens, and a mirror focus lens. Upon contact with the diverting structure, the first energy can be diverted within the energy guide 122A to one or more of the plasma generator 133 that is positioned near, but typically spaced apart from, the guide distal end 122D of the energy guide 122A, and the photoacoustic transducer 154 that is in optical communication with a side surface of the energy guide 122A. When utilized, the plasma generator 133 receives first energy emitted from the guide distal end 122D of the energy guide 122A to generate plasma in the catheter fluid 132 within the balloon interior 146, which, in turn, causes the creation of plasma bubbles and/or pressure waves that can be directed away from the side surface of the energy guide 122A and toward the balloon wall 130. Additionally, or in the alternative, when utilized, the photoacoustic transducer 154 converts light energy into an acoustic wave that extends away from the side surface of the energy guide 122A.

The source manifold 136 can be positioned at or near the proximal portion 114 of the catheter system 100. The source manifold 136 can include one or more proximal end openings that can receive the one or more energy guides 122A of the energy guide bundle 122, the guidewire 112, and/or an inflation conduit 140 that is coupled in fluid communication with the fluid pump 138. The catheter system 100 can also include the fluid pump 138 that is configured to inflate the balloon 104 with the catheter fluid 132 as needed.

As noted above, in the embodiment illustrated in FIG. 1, the system console 123 includes one or more of the energy source 124, the power source 125, the system controller 126, the GUI 127, and the contact detector assembly 142. Alternatively, the system console 123 can include more components or fewer components than those specifically illustrated in FIG. 1. For example, in certain non-exclusive alternative embodiments, the system console 123 can be designed without the GUI 127. Still alternatively, one or more of the energy source 124, the power source 125, the system controller 126, the GUI 127, and the contact detector assembly 142 can be provided within the catheter system 100 without the specific need for the system console 123.

As illustrated in FIG. 1, in certain embodiments, at least a portion of the diameter correlation system 147 and the contact detector assembly 142 can be positioned substantially within the system console 123. Alternatively, components of the diameter correlation system 147 and/or the contact detector assembly 142 can be positioned in a different manner than what is specifically shown in FIG. 1.

As shown, the system console 123, and the components included therewith, is operatively coupled to the catheter 102, the energy guide bundle 122, and the remainder of the catheter system 100. For example, in some embodiments, as illustrated in FIG. 1, the system console 123 can include a console connection aperture 148 (also sometimes referred to generally as a “socket”) by which the energy guide bundle 122 is mechanically coupled to the system console 123. In such embodiments, the energy guide bundle 122 can include a guide coupling housing 150 (which can generally include one or more ferrules) that houses a portion, such as the guide proximal end 122P, of each of the energy guides 122A. At least a portion of the guide coupling housing 150 is configured to fit and be selectively retained within the console connection aperture 148 to provide the mechanical coupling between the energy guide bundle 122 and the system console 123, as well as helping to provide an optical coupling between the energy source 124 and the energy guides 122A of the energy guide bundle 122.

The energy guide bundle 122 can also include a guide bundler 152 (or “shell”) that brings each of the individual energy guides 122A closer together so that the energy guides 122A and/or the energy guide bundle 122 can be in a more compact form as it extends as part of the catheter 102 into the blood vessel 108 during use of the catheter system 100.

The energy source 124 can be selectively and/or alternatively coupled in optical communication with each of the energy guides 122A, such as to the guide proximal end 122P of each of the energy guides 122A, in the energy guide bundle 122. In particular, the energy source 124 is configured to generate first energy in the form of a source beam 124A, such as a pulsed source beam, that can be selectively and/or alternatively directed to and received by each of the energy guides 122A in the energy guide bundle 122 as an individual guide beam 124B. Alternatively, the catheter system 100 can include more than one energy source 124. For example, in one non-exclusive alternative embodiment, the catheter system 100 can include a separate energy source 124 for each of the energy guides 122A in the energy guide bundle 122.

The energy source 124 can have any suitable design. In certain embodiments, the energy source 124 can be configured to provide sub-millisecond pulses of first energy from the energy source 124 that are focused onto a small spot in order to couple it into the guide proximal end 122P of the energy guide 122A. Such pulses of first energy are then directed and/or guided along the energy guides 122A to a location within the balloon interior 146 of the balloon 104, thereby inducing plasma formation (also sometimes referred to herein as a “plasma flash”) in the catheter fluid 132 within the balloon interior 146 of the balloon 104, such as via the plasma generator 133 that can include and/or incorporate a structure that is located at or near the guide distal end 122D of the energy guide 122A. In many embodiments, the plasma generator 133 can be positioned slightly spaced apart from the guide distal end 122D of the energy guide 122A. In certain embodiments, the plasma generator 133 can be provided in the form of a backstop-type structure with an angled face that redirects energy emitted from the guide distal end 122D toward the balloon wall 130 of the balloon 104 and/or toward the vessel wall 108A of the blood vessel 108 at the treatment site 106.

In particular, the first energy emitted at the guide distal end 122D of the energy guide 122A is directed toward and impinges on and energizes material of the plasma generator 133, such as material on an angled face of the plasma generator 133, for purposes of generating plasma in the catheter fluid 132 within the balloon interior 146. The plasma generation ionizes and superheats the surrounding catheter fluid 132 and thus causes rapid inertial bubble formation, and imparts pressure waves upon the treatment site 106. An exemplary plasma-induced bubble 134 is illustrated in FIG. 1.

The plasma generator 133 can be formed from any suitable materials. For example, in certain non-exclusive embodiments, the plasma generator 133 can be formed from one or more metallics and/or metal alloys having relatively high melting temperatures, such as titanium, stainless steel, tungsten, tantalum, platinum, molybdenum, niobium, iridium, etc. Alternatively, the plasma generator 133 can be formed from at least one of magnesium oxide, beryllium oxide, tungsten carbide, titanium nitride, titanium carbonitride, and titanium carbide. Still alternatively, the plasma generator 133 can be formed from at least one of diamond CVD and diamond. In other embodiments, the plasma generator 133 can be formed from a transition metal, an alloy metal, or a ceramic material. Yet alternatively, in some embodiments, the plasma generator 133 can be formed at least partially from a polymer, a polymeric material, and/or a plastic such as polyimide and nylon. Still alternatively, the plasma generator 133 can be formed from any other suitable materials.

In various non-exclusive alternative embodiments, the sub-millisecond pulses of first energy from the energy source 124 can be delivered to the treatment site 106 at a frequency of between approximately one hertz (Hz) and 5000 Hz, between approximately 30 Hz and 1000 Hz, between approximately ten Hz and 100 Hz, or between approximately one Hz and 30 Hz. Alternatively, the sub-millisecond pulses of first energy can be delivered to the treatment site 106 at a frequency that can be greater than 5000 Hz or less than one Hz, or any other suitable range of frequencies.

It is appreciated that although the energy source 124 is typically utilized to provide pulses of first energy, the energy source 124 can still be described as providing a single source beam 124A, i.e. a single pulsed source beam.

The energy sources 124 suitable for use can include various types of light sources including lasers and lamps. For example, in certain non-exclusive embodiments, the energy source 124 can be an infrared laser that emits first energy in the form of pulses of infrared light. Alternatively, as noted above, the energy sources 124 can include any suitable type of energy source.

Suitable lasers can include short pulse lasers on the sub-millisecond timescale. In some embodiments, the energy source 124 can include lasers on the nanosecond (ns) timescale. The lasers can also include short pulse lasers on the picosecond (ps), femtosecond (fs), and microsecond (μs) timescales. It is appreciated that there are many combinations of laser wavelengths, pulse widths and energy levels that can be employed to achieve plasma in the catheter fluid 132 of the catheter 102. In various non-exclusive alternative embodiments, the pulse widths can include those falling within a range including from at least ten ns to 3000 ns, at least 20 ns to 100 ns, or at least one ns to 500 ns. Alternatively, any other suitable pulse width range can be used.

Exemplary nanosecond lasers can include those within the UV to IR spectrum, spanning wavelengths of about ten nanometers (nm) to one millimeter (mm). In some embodiments, the energy sources 124 suitable for use in the catheter systems 100 can include those capable of producing light at wavelengths of from at least 750 nm to 2000 nm. In other embodiments, the energy sources 124 can include those capable of producing light at wavelengths of from at least 700 nm to 3000 nm. In still other embodiments, the energy sources 124 can include those capable of producing light at wavelengths of from at least 100 nm to ten micrometers (μm). Nanosecond lasers can include those having repetition rates of up to 200 kHz.

In some embodiments, the laser can include a Q-switched thulium:yttrium-aluminum-garnet (Tm:YAG) laser. In other embodiments, the laser can include a neodymium:yttrium-aluminum-garnet (Nd:YAG) laser, holmium:yttrium-aluminum-garnet (Ho:YAG) laser, erbium:yttrium-aluminum-garnet (Er:YAG) laser, excimer laser, helium-neon laser, carbon dioxide laser, as well as doped, pulsed, fiber lasers.

In still other embodiments, the energy source 124 can include a plurality of lasers that are grouped together in series. In yet other embodiments, the energy source 124 can include one or more low energy lasers that are fed into a high energy amplifier, such as a master oscillator power amplifier (MOPA). In still yet other embodiments, the energy source 124 can include a plurality of lasers that can be combined in parallel or in series to provide the energy needed to create the plasma bubble 134 in the catheter fluid 132.

The catheter system 100 can generate pressure waves having maximum pressures in the range of at least one megapascal (MPa) to 100 MPa. The maximum pressure generated by a particular catheter system 100 will depend on the energy source 124, the absorbing material, the bubble expansion, the propagation medium, the balloon material, and other factors. In various non-exclusive alternative embodiments, the catheter systems 100 can generate pressure waves having maximum pressures in the range of at least approximately two MPa to 50 MPa, at least approximately two MPa to 30 MPa, or at least approximately 15 MPa to 25 MPa.

The pressure waves can be imparted upon the treatment site 106 from a distance within a range from at least approximately 0.1 millimeters (mm) to greater than approximately 25 mm extending radially from the energy guides 122A when the catheter 102 is placed at the treatment site 106. In various non-exclusive alternative embodiments, the pressure waves can be imparted upon the treatment site 106 from a distance within a range from at least approximately ten mm to 20 mm, at least approximately one mm to ten mm, at least approximately 1.5 mm to four mm, or at least approximately 0.1 mm to ten mm extending radially from the energy guides 122A when the catheter 102 is placed at the treatment site 106. In other embodiments, the pressure waves can be imparted upon the treatment site 106 from another suitable distance that is different than the foregoing ranges. In some embodiments, the pressure waves can be imparted upon the treatment site 106 within a range of at least approximately two MPa to 30 MPa at a distance from at least approximately 0.1 mm to ten mm. In some embodiments, the pressure waves can be imparted upon the treatment site 106 from a range of at least approximately two MPa to 25 MPa at a distance from at least approximately 0.1 mm to ten mm. Still alternatively, other suitable pressure ranges and distances can be used.

The power source 125 is electrically coupled to and is configured to provide necessary power to each of the energy source 124, the system controller 126, the GUI 127, the handle assembly 128, the pressure sensor assembly 141, and the contact detector assembly 142. The power source 125 can have any suitable design for such purposes.

The system controller 126 is electrically coupled to and receives power from the power source 125. The system controller 126 is coupled to and is configured to control operation of each of the energy source 124, the GUI 127, the pressure sensor assembly 141, and the contact detector assembly 142. The system controller 126 can include one or more processors or circuits for purposes of controlling the operation of at least the energy source 124, the GUI 127, the pressure sensor assembly 141, and the contact detector assembly 142. For example, the system controller 126 can control the energy source 124 for generating pulses of first energy as desired and/or at any desired firing rate. The system controller 126 can also control the internal balloon pressure in conjunction with the pressure sensor assembly 141 and the balloon compliance chart 955 to control and/or determine the outer diameter 104BD of the balloon 104, for example, as part of the balloon diameter determination system 957. The system controller 126 can further control and/or operate in conjunction with the contact detector assembly 142 to effectively provide real-time continuous monitoring of the positioning of the balloon 104 relative to the vessel wall 108A of the blood vessel 108, so as to ensure that appropriate and desired contact exists between the balloon wall 130 and the vessel wall 108A prior to sending the first energy from the energy source 124 through the energy guides 122A and into the balloon interior 146 for purposes of treating and/or disrupting the intravascular lesions 106A at the treatment site 106.

In various embodiments, the system controller 126 can also be configured to control and/or monitor the algorithm that controls operation of the diameter correlation system 147. In certain embodiments, the algorithm of the diameter correlation system 147 can be incorporated substantially within the system controller 126.

The system controller 126 can further be configured to control operation of other components of the catheter system 100 such as the positioning of the catheter 102 and/or the guide distal end 122D of the energy guides 122A adjacent to the treatment site 106, the inflation of the balloon 104 with the catheter fluid 132, etc. Further, or in the alternative, the catheter system 100 can include one or more additional controllers that can be positioned in any suitable manner for purposes of controlling the various operations of the catheter system 100. For example, in certain embodiments, an additional controller and/or a portion of the system controller 126 can be positioned and/or incorporated within the handle assembly 128.

The GUI 127 is accessible by the user or operator of the catheter system 100. The GUI 127 is electrically connected to the system controller 126. With such design, the GUI 127 can be used by the user or operator to ensure that the catheter system 100 is effectively utilized to impart pressure onto and induce fractures at the treatment site(s) 106. The GUI 127 can provide the user or operator with information that can be used before, during and after use of the catheter system 100. For example, the GUI 127 can be utilized to transmit information to the user or operator that has been learned through operation of the diameter correlation system 147, including information from the pressure sensor assembly 141, the contact detector assembly 142, the balloon diameter determination system 957, the vessel diameter determination system 958, and the vessel diameter mapping system 959. In one embodiment, the GUI 127 can provide static visual data and/or information to the user or operator. In addition, or in the alternative, the GUI 127 can provide dynamic visual data and/or information to the user or operator, such as video data or any other data that changes over time during use of the catheter system 100. In various embodiments, the GUI 127 can include one or more colors, different sizes, varying brightness, etc., that may act as alerts to the user or operator. Additionally, or in the alternative, the GUI 127 can provide audio data or information to the user or operator. The specifics of the GUI 127 can vary depending upon the design requirements of the catheter system 100, or the specific needs, specifications and/or desires of the user or operator.

As shown in FIG. 1, the handle assembly 128 can be positioned at or near the proximal portion 114 of the catheter system 100, and/or near the source manifold 136. In this embodiment, the handle assembly 128 is coupled to the balloon 104 and is positioned spaced apart from the balloon 104. Alternatively, the handle assembly 128 can be positioned at another suitable location.

The handle assembly 128 is handled and used by the user or operator to operate, position, and control the catheter 102. The design and specific features of the handle assembly 128 can vary to suit the design requirements of the catheter system 100. In the embodiment illustrated in FIG. 1, the handle assembly 128 is separate from, but in electrical and/or fluid communication with one or more of the system controller 126, the energy source 124, the fluid pump 138, the GUI 127, the pressure sensor assembly 141, and the contact detector assembly 142.

In some embodiments, the handle assembly 128 can integrate and/or include at least a portion of the system controller 126 within an interior of the handle assembly 128. For example, as shown, in certain such embodiments, the handle assembly 128 can include circuitry 156, which is electrically coupled between catheter electronics and the system console 123, and which can form at least a portion of the system controller 126. In some embodiments, the circuitry 156 can receive electrical signals or data from the pressure sensor assembly 141 and/or the contact detector assembly 142. Further, or in the alternative, the circuitry 156 can transmit such electrical signals or otherwise provide data to the system controller 126.

In one embodiment, the circuitry 156 can include a printed circuit board having one or more integrated circuits, or any other suitable circuitry. In an alternative embodiment, the circuitry 156 can be omitted, or can be included within the system controller 126, which in various embodiments can be positioned outside of the handle assembly 128, such as within the system console 123. It is understood that the handle assembly 128 can include fewer or additional components than those specifically illustrated and described herein.

The pressure sensor assembly 141 includes one or more pressure sensors 141S that are configured to sense and/or monitor the internal balloon pressure of the catheter fluid 132 that is retained within the balloon interior 146 of the balloon 104 during operation of the catheter system 100. More specifically, the pressure sensor assembly 141 and/or the pressure sensors 141S can provide real-time continuous monitoring of the internal balloon pressure within the balloon interior 146 in conjunction with electronics or optics that can be included in the handle assembly 128 and/or the system console 123. The pressure sensor assembly 141 and/or the pressure sensors 141S can generate a sensor signal or sensor output relevant to the sensed internal balloon pressure and provide such sensor output to the system controller 126 that is configured to control various operations of the catheter system 100. The system controller 126 can control the internal balloon pressure for purposes of controlling the outer diameter 104BD of the balloon 104, through use of the balloon compliance chart 955 for the particular style, size, design, material, etc. of the balloon 104 in use within the catheter system 100. This sensing and/or monitoring of the internal balloon pressure with the pressure sensor assembly 141 and/or the pressure sensors 141S thus provides valuable information to the user or operator as to the performance, reliability, and safety of the catheter system 100. For example, the sensed internal balloon pressure can be utilized in conjunction with the balloon compliance chart 955, which plots the outer diameter of the balloon versus the internal balloon pressure, to determine the outer diameter 104BD of the balloon 104. The outer diameter 104BD of the balloon 104 can then be displayed on the GUI 127, therefore allowing the user or operator to know the outer diameter 104BD of the balloon 104 during the procedure.

It is appreciated that the one or more pressure sensors 141S of the pressure sensor assembly 141 can be positioned at any suitable locations within the catheter system 100 so that they are in fluid communication with the catheter fluid 132 within the balloon interior 146. For example, in certain non-exclusive embodiments, as shown, the one or more pressures sensors 141S of the pressure sensor assembly 141 can be positioned at one or more of within the handle assembly 128, within the catheter shaft 110, within the balloon interior 146 of the balloon 104, within the fluid pump 138, and within or along the inflation conduit 140, in order to effectively sense and/or monitor the internal balloon pressure within the balloon interior 146 of the balloon 104. Alternatively, the one or more pressure sensors 141S can be positioned in any other suitable location along the fluid path of the catheter fluid 132 that is utilized to selectively inflate the balloon 104.

The one or more pressure sensors 141S can have any suitable design for purposes of sensing and/or monitoring the internal balloon pressure of the catheter fluid 132 that is retained within the balloon interior 146 of the balloon 104 during operation of the catheter system 100. For example, in certain non-exclusive embodiments, the one or more pressure sensors 141S can be selected from the group consisting of an optical fiber sensor, a diaphragm sensor and a MEMS sensor. Alternatively, the one or more pressure sensors 141S can have another suitable design.

In addition to being useful for determining the outer diameter 104BD of the balloon 104 as part of the diameter correlation system 147, the sensed internal balloon pressure that is sensed by the one or more pressure sensors 141S of the pressure sensor assembly 141 can also be utilized to address other challenges with the performance, reliability and safety of a catheter 102, in particular one that utilizes an energy source 124 to create a localized plasma which in turn induces a high energy bubble inside the balloon 104. For example, additional issues that can be addressed by the pressure sensor assembly 141 include, but are not limited to: (1) detection of rupturing or bursting of the balloon 104, (2) detection of successful firing of the plasma generator 133, (3) detection of failure of the plasma generator 133, and (4) monitoring of progression of the procedure and efficacy of treatment.

The contact detector assembly 142 is configured to effectively detect and/or monitor the positioning of the balloon wall 130 of the balloon 104 relative to the vessel wall 108A of the blood vessel 108 at the treatment site 106. More particularly, in various implementations of the contact detector assembly 142, the contact detector assembly 142 can be configured to detect 1) when the balloon wall 130 is in optimal contact with the vessel wall 108A at the treatment site 106 to optimize energy transmission and treatment efficacy, 2) when the balloon 104 is undersized for the blood vessel 108 under treatment and providing feedback to the user, and 3) any condition where balloon contact is suboptimal for treatment.

In many implementations, the catheter system 100 can work through multiple mechanisms. A first mechanism entails static loading of the vessel wall 108A through hydrostatic pressure applied through the balloon 104 at a nominal four atmospheres (atm) of pressure. Static loading and contact with the vessel wall 108A create an optimal path for transmission of acoustic energy from the plasma-driven energy source 124 through to the vascular lesion 106A at the treatment site 106. The static loading of the vessel wall 108A contributes to efficacy of the catheter system 100 as it does to efficiency in coupling acoustic energy. It is critical that the balloon 104 be sized appropriately for optimal operation of the catheter system 100.

A second mechanism entails coupling of acoustic energy from the localized plasma to the vascular lesion 106A. It is essential for the efficacy and performance of this type of catheter system 100 for the outer diameter 104BD of the balloon 104 to be sized to correctly fit to the inner diameter 108D of the blood vessel 108 under treatment. Ideally, the catheter system 100 and/or the contact detector assembly 142 will gauge the fit of the catheter 102 as the catheter 102 is inserted into the blood vessel 108 and provide feedback to the user about the adequacy of the fit of a fully expanded balloon 104 with the blood vessel 108 under treatment.

A balloon 104 that is the correct size for the blood vessel 108 under treatment will displace the blood 305 within the blood vessel 108 when the balloon 104 is fully inflated and the balloon wall 130 is forced into contact with the vessel wall 108A. Alternatively, a balloon 104 that is undersized for the blood vessel 108 under treatment will not fully displace the blood 305 in the blood vessel 108 when the balloon 104 is inflated. As a result, an annulus of blood 305 will remain between the balloon wall 130 and the vessel wall 108A. As described herein, it is possible to detect the presence or absence of blood 305 under these conditions through optical means. The absorption spectrums for blood 305 and the tissues comprising the vessel wall 108A are different across a wide range of wavelengths in the visible spectrum and near IR spectrum. This concept enables the contact detector assembly 142 to effectively diagnose how much blood 305 may be present between the balloon wall 130 and the vessel wall 108A, and thus whether or not optimal contact exists between the balloon wall 130 and the vessel wall 108A.

As described in detail herein, the primary fluid that is detected with the contact detector assembly 142 is blood 305. However, it is appreciated that the contact detector assembly 142 can be configured and utilized to detect any type(s) of fluid that may be present within the blood vessel 108, such as between the balloon wall 130 and the vessel wall 108A. For example, in certain implementations, there could be a mix of contrast medium and blood 305 or saline and blood 305. Thus, one skilled in the art would recognize that the contact detector assembly 142 could be configured by wavelength selection to detect any fluid or blood-fluid mixture.

It is also appreciated that the polymer comprising the balloon wall 130 of the balloon 104 is optically transparent across the noted range of wavelengths in the visible spectrum and near IR spectrum.

It is further appreciated that the contact detector assembly 142 can have any suitable design for purposes of effectively detecting whether or not optimal contact exists between the balloon wall 130 and the vessel wall 108A. Certain non-exclusive examples of potential designs and applications for the contact detector assembly 142 are described in detail herein below.

In many embodiments, the contact detector assembly 142 can include optical splitting and photodetection devices to provide a means for probing the space between the balloon wall 130 and the vessel wall 108A using the existing optics and hardware for high-energy delivery and plasma generation. For example, in some embodiments, the contact detector assembly 142 uses the energy guide 122A and the plasma generator 133 to deliver first light energy at selected wavelengths from the illumination sources 366, 368 to the space between the balloon wall 130 and the vessel wall 108A, and the distal light receiver 122R then collects light scattered back from there in the form of second light energy (also sometimes referred to as a “returning energy beam”) and return it to components of the contact detector assembly 142 for analysis to detect blood 305 or other fluids in the space being probed. Alternatively, in other embodiments, it is possible to implement the desired functionality of the contact detector assembly 142 using a separate energy guide and energy steering and collecting device. In such alternative embodiments, the separate energy guide utilized by the contact detector assembly 142 can be a light guide, whereas the original energy guide 122A can then, in some non-exclusive implementations, be replaced by any suitable type of energy guide, be it electrical, acoustic, pneumatic, mechanical, etc.

In most, if not all, implementations of the catheter system 100 including the contact detector assembly 142 having features of the present invention, a treatment procedure would be prevented or stopped if any suboptimal balloon wall 130 to vessel wall 108A conditions were detected by the contact detector assembly 142 so as to mitigate associated risks to the patient 109. In certain embodiments, this can entail locking out of the energy source 124 through use of a safety shutdown system 362 (illustrated in FIG. 3A), which in some such embodiments can include one or more of a safety interlock 362A (illustrated in FIG. 3A) and a shutter 362B (illustrated in FIG. 3A), that can be utilized in conjunction with the contact detector assembly 142. This provides a necessary safety interlock and mitigation for potentially hazardous conditions in which undesired risks to the patient 109 may exist. Moreover, the system controller 126 could be used to indicate to the operator, such as via the GUI 127, to halt any procedure and/or remove the catheter 102 from the patient 109 under treatment when such suboptimal conditions are found to exist. It is further appreciated that the safety interlock system 362 can also be employed during an initial step for use of the contact detector assembly 142, such that the energy source 124 can be locked out prior to the contact detector assembly 142 being utilized to determine whether optimal contact has been established between the balloon wall 130 of the balloon 104 and the vessel wall 108A of the blood vessel 108.

As with all embodiments illustrated and described herein, various structures may be omitted from the figures for clarity and ease of understanding. Further, the figures may include certain structures that can be omitted without deviating from the intent and scope of the invention.

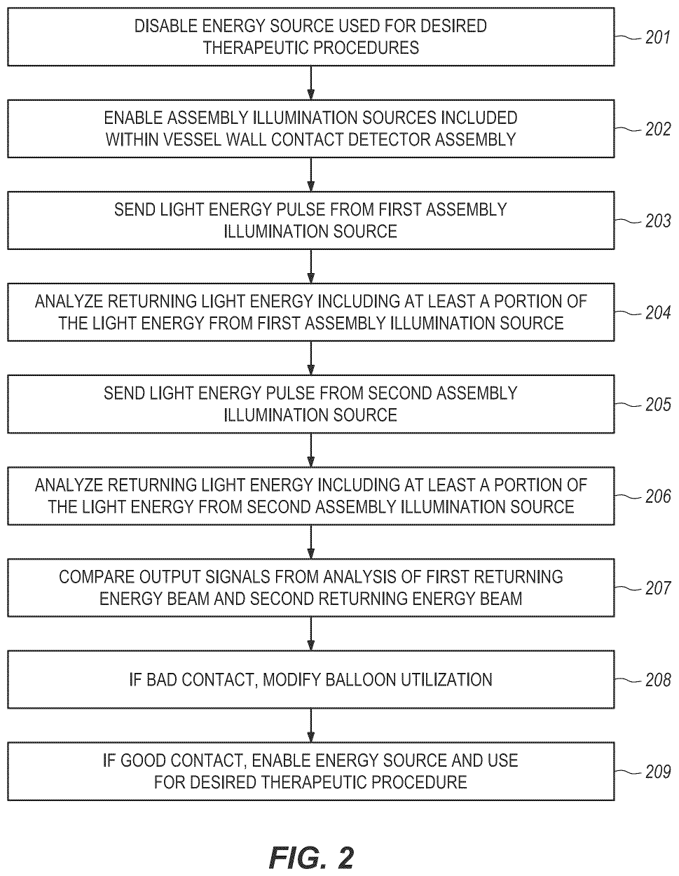

FIG. 2 is a flowchart illustrating a first operational mode for an embodiment of the catheter system including an embodiment of the vessel wall contact detector assembly. It is appreciated that any of the steps listed in the flowchart of FIG. 2 can be modified, deleted and/or combined in any suitable manner, and/or the order of steps can be changed, without deviating from the intended breadth and scope of the present invention. One or more steps can also be added to the series of steps specifically illustrated and described within the flowchart without deviating from the intended breadth and scope of the present invention.

Referring briefly to FIG. 3A, FIG. 3A is a simplified schematic view illustration of a portion of the embodiment of the catheter system 300 including the embodiment of the vessel wall contact detector assembly 342 usable within the first operational mode as described in relation to FIG. 2.

The design of the catheter system 300 and the contact detector assembly 342 can be varied. It is appreciated that various components of the catheter system 300, such as are shown in FIG. 1, are not illustrated in FIG. 3A for purposes of clarity and ease of illustration. However, it is appreciated that the catheter system 300 will likely include most, if not all, of such components.

In various embodiments, as illustrated in FIG. 3A, the catheter system includes one or more of a light source 324 (such as a pulsed infrared laser source in one non-exclusive embodiment, or another suitable energy source), a pulse generator 360 that is coupled to the light source 324, a safety shutdown system 362 including a safety interlock 362A and a shutter 362B, a first optical element 364 (such as a coupling lens in one non-exclusive embodiment), a light guide 322A (such as an optical fiber in one non-exclusive embodiment, or another suitable energy guide), a plasma generator 333, a balloon 304 including a balloon wall 330, and the contact detector assembly 342. As further shown in FIG. 3A, the contact detector assembly 342 can include one or more of a first assembly illumination source 366, a first source driver 366A, a second assembly illumination source 368, a second source driver 368A, a first redirector 370, a second redirector 372, a first beamsplitter 374, a filter 376, a second beamsplitter 378, a second optical element 380 (such as an imaging lens in one non-exclusive embodiment), a photodetector 382, an amplifier 384, and control electronics 386, which can include one or more processors or circuits. Alternatively, in other embodiments, the catheter system 300 and/or the contact detector assembly 342 can include more components or fewer components than those specifically noted above. Still alternatively, in still other embodiments, the various components of the catheter system 300 and/or the contact detector assembly 342 can be positioned in a different manner than what is specifically illustrated in FIG. 3A.

It is appreciated that the use of the terms “first assembly illumination source” and “second assembly illumination source” are used merely for convenience and ease of discussion. As such, it is further appreciated that either of the assembly illumination sources 366, 368 can be referred to as the “first assembly illumination source” and/or the “second assembly illumination source”. Additionally, it is also appreciated that the terms “first optical element” and “second optical element”, “first source driver” and “second source driver”, “first redirector” and “second redirector”, and “first beamsplitter” and “second beamsplitter”, can also be used interchangeably to identify either optical element 364, 380, either source driver 366A, 368A, either redirector 370, 372, and either beamsplitter 374, 378, respectively.

FIG. 3A also shows that, during use of the catheter system 300 and/or the contact detector assembly 342, the balloon wall 330 of the balloon 304 can be positioned (i) in a first condition 390A—in good and/or optimal contact with the vessel wall 308A of the blood vessel 308, such as with little or no blood 305 positioned between the balloon wall 330 and the vessel wall 308A, and/or (ii) in a second condition 390B—in bad and/or suboptimal contact with the vessel wall 308A of the blood vessel 308, such as with a relatively thick layer of blood 305 positioned between the balloon wall 330 and the vessel wall 308A. In various embodiments, as described herein, the contact detector assembly 342 is uniquely configured to establish whether the balloon wall 330 of the balloon 304 is positioned relative to the vessel wall 308A of the blood vessel 308 in the first condition 390A (with good or optimal contact between the balloon wall 330 and the vessel wall 308A) or in the second condition 390B (with bad or suboptimal contact between the balloon wall 330 and the vessel wall 308A).

In this embodiment, the contact detector assembly 342 includes a plurality of collimated assembly illumination sources 366, 368, each having a specific wavelength. More specifically, this approach involves using two separate assembly illumination sources 366, 368 with different wavelengths, one assembly illumination source that is highly transmitted by blood 305 and reflected by the vessel wall 308A, and the other assembly illumination source that is strongly reflected and/or scattered by blood 305. Alternatively, the contact detector assembly 342 can include only a single assembly illumination source. Still alternatively, the contact detector assembly 342 can include assembly illumination source(s) that include three or more specific wavelengths.

In certain embodiments, the assembly illumination sources 366, 368 could be diode lasers, high intensity LEDs with optics to collimate the emitted beam, or other solid-state light emitting sources. As noted, this embodiment of the invention uses two distinct wavelengths, one with a high absorption coefficient for blood 305 and one with a low absorption coefficient. Ideally, these will both be at isosbestic points for the absorption spectrums of O₂Hb and deO₂Hb so that the relative oxygenation of the blood 305 would not affect the relative absorption or transmission of the light energy. Possible wavelengths for the assembly illumination sources 366, 368 are described in greater detail below. It is further noted that although the preferred wavelengths may be at isosbestic points for the absorption spectrums of O₂Hb and deO₂Hb, it may not be necessary to have both wavelengths at isosbestic points.

Returning to FIG. 2, at step 201, the light source (or other suitable energy source) that is utilized within the catheter system for desired therapeutic procedures is disabled. The disabling of the light source is undertaken so that light energy from the light source is not directed into and through the light guide until after optimal contact between the balloon wall and the vessel wall has been appropriately established through use of the contact detector assembly.

It is appreciated that the step of disabling the light source can be accomplished in any suitable manner. For example, in certain implementations, the light source can be disabled through use of the safety interlock system, which can employ the safety interlock and/or the shutter, through specific disabling of the pulse generator that is coupled to the light source, and/or through otherwise blocking power from being supplied to the light source.

For example, referring again briefly to FIG. 3A, prior to usage of the catheter system 300 as part of a therapeutic procedure, the control electronics 386 can be configured to send a signal to the safety shutdown system 362 to disable operation of the light source 324. More particularly, in one embodiment, the signal from the control electronics 386 to the safety shutdown system 362 can be used to activate the safety interlock 362A, which blocks any signal from the pulse generator 360 to the light source 324, thus effectively stopping any generation of light pulses from the light source 324. Additionally, or in the alternative, the signal from the control electronics 386 to the safety shutdown system 362 can be used to activate the shutter 362B which can be closed, thereby blocking any light pulses from the light source 324 that would otherwise be directed toward and coupled into the light guide 322A. With such safety shutdown system 386, usage of the catheter system 300 when there is suboptimal contact between the balloon wall 330 and the vessel wall 308A is effectively inhibited.

Returning again to FIG. 2, at step 202, the first assembly illumination source and the second illumination source included within the contact detector assembly are enabled.

At step 203, a first light energy pulse from the first assembly illumination source is generated in the form of a first assembly illumination beam, and is transmitted toward and through the light guide into the balloon interior. The first light energy of the first assembly illumination beam can then be redirected, such as by the plasma generator in one embodiment, toward the balloon wall of the balloon and/or toward the vessel wall of the blood vessel.

Referring now to FIG. 3B, FIG. 3B is a simplified schematic view illustration of the portion of the embodiment of the catheter system 300 including the embodiment of the vessel wall contact detector assembly 342 illustrated in FIG. 3A, with step 203 within the first operational mode being illustrated.

In particular, as illustrated, the first source driver 366A activates the first assembly illumination source 366 to generate a pulse of first light energy in the form of a first assembly illumination beam 366B that is directed from the first assembly illumination source 366 toward the light guide 322A. The desired wavelength of the first assembly illumination beam 366B can be varied. In particular, the wavelength range useful for blood and vessel wall detection spans the full visible range down into near IR. It is bounded in this approach at short wavelengths by transmission limits for the optical fiber towards the UV, which is around 250 nm for IR-fused silica. It is limited at long wavelengths by the absorption spectrum of biological materials and the bandgap of solid-state photodetectors which is 1.1 μm for silicon. Use of more esoteric materials and detectors could extend this range. For example, UV-fused silica could be usable down to 180 nm, and InGaAs photodiodes can detect light out to 1.68 μm. The absorption spectrum for biological materials involved ultimately become limiting factors. As mentioned earlier, it is preferable to use wavelengths at the isosbestic points of Hemoglobin, though this is not essential to the functionality of the method. An isosbestic point is a specific wavelength at which the total absorbance of a sample does not change during a chemical reaction or a physical change of the sample. In this case, the change from deO₂Hb to its oxygenated form, O₂Hb. There are eleven isosbestic points for deO₂Hb and O₂Hb over the 200-900 nm wavelength range: 255, 350, 390, 422, 452, 500, 529, 545, 570, 584, and 805 nm.

In certain non-exclusive embodiments, the first assembly illumination beam 366B can be at a wavelength that is generally strongly scattered and/or reflected when it comes into contact with blood 305, such as approximately 550 nm and 600 nm. Alternatively, the first assembly illumination beam 366B can be at another suitable wavelength.

The first assembly illumination beam 366B is initially directed toward and is redirected by the first redirector 370, such as a mirror in one non-exclusive embodiment, which in certain implementations can redirect the first assembly illumination beam 366B by approximately ninety degrees. The first assembly illumination beam 366B is then directed through the second redirector 372, such as a dichroic mirror that transmits light at certain wavelengths and redirects light at other wavelengths, before impinging upon the first beamsplitter 374.

At least a portion of the first assembly illumination beam 366B is transmitted by the first beamsplitter 374, which can be a non-polarizing variable beamsplitter such as a 50/50 R/T in certain non-exclusive embodiments. In one embodiment, the first assembly illumination source 366 is a diode laser with high intensity so enabling a low transmission beamsplitter to be used, such as 90/10; this would allow a much greater return signal for the contact detector assembly 342. It would also be possible to use a polarizing beamsplitter and polarize the first assembly illumination beam 366B from the first assembly illumination source 366 for 100% transmission.

The portion of the first assembly illumination beam 366B that has been transmitted by the first beamsplitter 374 then passes through the filter 376, such as a short pass filter in one non-exclusive embodiment. As such, the illumination and detection systems of the contactor assembly 342 are isolated from the high-energy light source 324 using the filter 376. This would eliminate any back-reflected light during pulsed plasma operation, improving SNR for the photodetector 382 to enable the contact detector assembly 342 to operate when the catheter system 300 is in therapeutic mode.

After passing through the filter 376, the remaining portion of the first assembly illumination beam 366B then impinges upon the second beamsplitter 378, which can be a dichroic beamsplitter in certain embodiments that transmits light at certain wavelengths and redirects light at other wavelengths. In certain embodiments, the second beamsplitter 378 is configured to pass or transmit light with wavelengths longer than those visible to the photodetector 382. Such threshold wavelength can be referred to as the cutoff wavelength. The second beamsplitter 378 is further configured to reflect all light having a wavelength that is shorter than the cutoff wavelength. It is appreciated that the first assembly illumination beam 366B will be at such a wavelength that it will be reflected substantially in its entirety by the second beamsplitter 378.

As shown, the remaining portion of the first assembly illumination beam 366B is redirected by the second beamsplitter 378 before passing through the first optical element 364 which collimates the first assembly illumination beam 366B and focuses the first light energy from the portion of the first assembly illumination beam 366B onto a guide proximal end 322P of the light guide 322A. The first light energy from the portion of the first assembly illumination beam 366B is guided through the light guide 322A and is emitted at a guide distal end 322D of the light guide 322A before it impinges upon the plasma generator 333. In certain embodiments, the plasma generator 333 can be provided in the form of a backstop-type structure with an angled face 333F that redirects the first light energy toward the balloon wall 330 of the balloon 304 and/or the vessel wall 308A of the blood vessel 308 at the treatment site 306. Thus, the angled face 333F of the plasma generator 333 acts like a single surface mirror. The beam spot of the first assembly illumination beam 366B at the surface of the angled face 333F is narrow due to the low numerical aperture of the light guide 322A when immersed in the catheter fluid 132 (illustrated in FIG. 1) and will, in turn, form a highly concentrated spot on the balloon wall 330 and nearby tissue, such as the vessel wall 308A of the blood vessel 308. As described in greater detail herein below, this intense spot will scatter from the blood 305 or vessel wall 308A and be directed back toward the guide distal end 322D of the light guide 322A by the angled face 333F where it will be collected within the numerical aperture of the guide distal end 322D of the light guide 322A.

As shown in FIG. 3B, the first light energy from the portion of the first assembly illumination beam 366B is generally reflected when it contacts the blood 305 whether the balloon 304 is in a good contact condition 390A or in a bad contact condition 390B.

Returning again to FIG. 2, at step 204, at least a portion of the first light energy from the first assembly illumination source is reflected and/or scattered back from the blood, is guided back through the light guide, and is subsequently analyzed by the contact detector assembly.

Referring now to FIG. 3C, FIG. 3C is a simplified schematic view illustration of the portion of the embodiment of the catheter system 300 including the embodiment of the vessel wall contact detector assembly 342 illustrated in FIG. 3A, with step 204 within the first operational mode being illustrated.

In particular, as illustrated, a portion of the first light energy from the first assembly illumination source 366 that is incident on the balloon wall 330 is reflected and/or scattered back from the blood 305 as second light energy and/or a first returning energy beam 366R that is received by the distal light receiver 322R that couples the second light energy and/or the first returning energy beam 366R into the guide distal end 322D of the light guide 322A. In certain embodiments, the angled face 333F of the plasma generator 333 can collect the second light energy reflected and/or scattered back from the blood 305, and redirect the second light energy back into the guide distal end 322D of the light guide 322A. Thus, the angled face 333F of the plasma generator 333 acts like a single surface mirror and can function as at least a portion of the distal light receiver 322R.

The second light energy and/or the first returning energy beam 366R is then emitted from the guide proximal end 322P from where it is directed through the first optical element 364, which collimates the first returning energy beam 366R. The collimated first returning energy beam 366R is then directed to the second beamsplitter 378, such as the dichroic beamsplitter.

Due to the specific wavelength of the first returning energy beam 366R, at least a portion of the first returning energy beam 366R is redirected by the second beamsplitter 378 and then is directed through the filter 376 before it impinges on the first beamsplitter 374. Stated in another manner, the first beamsplitter 374 is positioned in the optical path of the first returning energy beam 366R reflected from the second beamsplitter 378. This creates a two-way system for probing the treatment site 306 through the light guide 322A and detection of conditions there by the second light energy returned in the form of the first returning energy beam 366R. The two paths provide one to transmit the first assembly illumination beam 366B (illustrated in FIG. 3B) into the guide proximal end 322P of the light guide 322A and one to collect the second light energy of the first returning energy beam 366R returning from the guide distal end 322D.

At least a portion of the first returning energy beam 366R is then redirected by the first beamsplitter 374 through the second optical element 380 and onto the photodetector 382. The second optical element 380 focuses the collimated first returning energy beam 366R, forming an image of the end face of the light guide 322A onto the photodetector 382, thereby coupling light emitted from the guide proximal end 322P of the light guide 322A. In certain non-exclusive alternative embodiments, the photodetector 382 can be a photodiode, an area sensor such as a CCD or CMOS camera, or a spectrophotometer. In suitable arrangements the chain of optical elements 364, 380 can create a high-resolution image of the end face of the light guide 322A on an image sensor.

The photodetector 382 then generates a signal based on the strength of the light at the specific wavelength. In some embodiments, the photodetector 382 generates a signal that is based on the second light energy or first returning energy beam 366R reflected and/or scattered back from the blood 305 that has been received by the distal light receiver 322R at or near the guide distal end 322D of the light guide 322A and that has been collected by the photodetector 382.

The signal from the photodetector 382 is directed toward the amplifier 384 where the signal from the photodetector 382 is amplified before being sent to the control electronics 386 for processing and analysis. Alternatively, in other embodiments, the contact detector assembly 342 can be designed without the amplifier 384. In such alternative embodiments, the signal from the photodetector 382 can be sent to the control electronics 386 for processing and analysis. In either embodiment, it can be stated that the signal from the photodetector 382 is being used by the control electronics 386 to determine the contact condition between the balloon wall 330 and the vessel wall 308A (illustrated in FIG. 3A).

In some embodiments, the control electronics 386 can be included as part of the system controller 126 (illustrated in FIG. 1). Alternatively, the control electronics 386 can be provided independently of the system controller 126 and can be in electrical communication with the system controller 126.

Returning again to FIG. 2, at step 205, a first light energy pulse from the second assembly light source is generated in the form of a second assembly illumination beam, and is transmitted through the light guide into the balloon interior. The first light energy of the second assembly illumination beam can then be redirected, such as by the plasma generator in one embodiment, toward the balloon wall of the balloon and/or toward the vessel wall of the blood vessel.

Referring now to FIG. 3D, FIG. 3D is a simplified schematic view illustration of the portion of the embodiment of the catheter system 300 including the embodiment of the vessel wall contact detector assembly 342 illustrated in FIG. 3A, with step 205 within the first operational mode being illustrated.

In particular, as illustrated, the second source driver 368A activates the second assembly illumination source 368 to generate a pulse of first light energy in the form of a second assembly illumination beam 368B that is directed from the second assembly illumination source 368 toward the light guide 322A. The desired wavelength of the second assembly illumination beam 368B can be varied. In particular, the wavelength range useful for blood and vessel wall detection spans the full visible range down into near IR. It is bounded in this approach at short wavelengths by transmission limits for the optical fiber towards the UV, which is around 250 nm for IR-fused silica. It is limited at long wavelengths by the absorption spectrum of biological materials and the bandgap of solid-state photodetectors which is 1.1 μm for silicon. Use of more esoteric materials and detectors could extend this range. For example, UV-fused silica could be usable down to 180 nm, and InGaAs photodiodes can detect light out to 1.68 μm. The absorption spectrum for biological materials involved ultimately become limiting factors. As mentioned earlier, it is preferable to use wavelengths at the isosbestic points of Hemoglobin, though this is not essential to the functionality of the method. An isosbestic point is a specific wavelength at which the total absorbance of a sample does not change during a chemical reaction or a physical change of the sample. In this case, the change from deO₂Hb to its oxygenated form, O₂Hb. There are eleven isosbestic points for deO₂Hb and O₂Hb over the 200-900 nm wavelength range: 255, 350, 390, 422, 452, 500, 529, 545, 570, 584, and 805 nm.

In certain non-exclusive embodiments, the second assembly illumination beam 368B can be at a wavelength that passes through and/or is transmitted by the blood 305 and then is generally reflected when it comes into contact with vessel wall 308A, such as approximately 640 nm and 750 nm. Alternatively, the second assembly illumination beam 368B can be at another suitable wavelength.

The second assembly illumination beam 368B is initially directed toward and is redirected by the second redirector 372, such as a dichroic mirror that transmits light at certain wavelengths and redirects light at other wavelengths, which in certain embodiments can redirect the second assembly illumination beam 368B by approximately ninety degrees. The second assembly illumination beam 368B is then directed toward and impinges upon the first beamsplitter 374.

At least a portion of the second assembly illumination beam 368B is transmitted by the first beamsplitter 374, which can be a non-polarizing variable beamsplitter such as a 50/50 R/T in certain non-exclusive embodiments. In one embodiment, the second assembly illumination source 368 is a diode laser with high intensity so enabling a low transmission beamsplitter to be used, such as 90/10; this would allow a much greater return signal for the contact detector assembly 342. It would also be possible to use a polarizing beamsplitter and polarize the second assembly illumination beam 368B from the second assembly illumination source 368 for 100% transmission.

The portion of the second assembly illumination beam 368B that has been transmitted by the first beamsplitter 374 then passes through the filter 376 before impinging upon the second beamsplitter 378, which can be a dichroic beamsplitter in certain embodiments that transmits light at certain wavelengths and redirects light at other wavelengths. In certain embodiments, the second beamsplitter 378 is configured to pass or transmit light with wavelengths longer than those visible to the photodetector 382. Such threshold wavelength can be referred to as the cutoff wavelength. The second beamsplitter 378 is further configured to reflect all light having a wavelength that is shorter than the cutoff wavelength. It is appreciated that the second assembly illumination beam 368B will be at such a wavelength that it will be reflected substantially in its entirety by the second beamsplitter 378.

As shown, the remaining portion of the second assembly illumination beam 368B is redirected by the second beamsplitter 378 before passing through the first optical element 364 which collimates the second assembly illumination beam 368B and focuses the first light energy from the portion of the second assembly illumination beam 368B onto the guide proximal end 322P of the light guide 322A. The first light energy from the portion of the second assembly illumination beam 368B is guided through the light guide 322A and is emitted at the guide distal end 322D of the light guide 322A before it impinges upon the plasma generator 333. In certain embodiments, the angled face 333F of the plasma generator 333 redirects the first light energy of the second assembly illumination beam 368B toward the balloon wall 330 of the balloon 304 and/or the vessel wall 308A of the blood vessel 308 at the treatment site 306. Thus, the angled face 333F of the plasma generator 333 acts like a single surface mirror. The beam spot of the second assembly illumination beam 368B at the surface of the angled face 333F is narrow due to the low numerical aperture of the light guide 322A when immersed in the catheter fluid 132 (illustrated in FIG. 1) and will, in turn, form a highly concentrated spot on the balloon wall 330 and nearby tissue, such as the vessel wall 308A of the blood vessel 308. As described in greater detail herein below, this intense spot will be transmitted by the blood 305 and will scatter from the vessel wall 308A and be directed back toward the guide distal end 322D of the light guide 322A by the angled face 333F of the plasma generator 333 where it will be collected within the numerical aperture of the guide distal end 322D of the light guide 322A.

As shown in FIG. 3D, the first light energy from the portion of the second assembly illumination beam 368B passes through the blood 305 and then is generally reflected when it contacts the vessel wall 308A whether the balloon 304 is in a good contact condition 390A or in a bad contact condition 390B.

Returning again to FIG. 2, at step 206, at least a portion of the first light energy from the second assembly illumination source is reflected and/or scattered back from the vessel wall, is guided back through the light guide, and is subsequently analyzed by the contact detector assembly.

Referring now to FIG. 3E, FIG. 3E is a simplified schematic view illustration of the portion of the embodiment of the catheter system 300 including the embodiment of the vessel wall contact detector assembly 342 illustrated in FIG. 3A, with step 206 within the first operational mode being illustrated.

In particular, as illustrated, a portion of the first light energy from the second assembly illumination source 368 is reflected and/or scattered back from the vessel wall 308A as second light energy and/or a second returning energy beam 368R that is received by the distal light receiver 322R that couples the second light energy and/or the second returning energy beam 368R into the guide distal end 322D of the light guide 322A. In certain embodiments, the angled face 333F of the plasma generator 333 can collect the second light energy reflected and/or scattered back from the vessel wall 308A, and redirect the second light energy back into the guide distal end 322D of the light guide 322A. Thus, the angled face 333F of the plasma generator 333 acts like a single surface mirror and can function as at least a portion of the distal light receiver 322R.

The second light energy and/or the second returning energy beam 368R is then emitted from the guide proximal end 322P from where it is directed through the first optical element 364, which collimates the second returning energy beam 368R. The collimated second returning energy beam 368R is then directed to the second beamsplitter 378, such as the dichroic beamsplitter.

Due to the specific wavelength of the second returning energy beam 368R, at least a portion of the second returning energy beam 368R is redirected by the second beamsplitter 378 and then is directed through the filter 376 before it impinges on the first beamsplitter 374. Stated in another manner, the first beamsplitter 374 is positioned in the optical path of the second returning energy beam 368R reflected from the second beamsplitter 378. This creates a two-way system for probing the treatment site 306 through the light guide 322A and detection of conditions there by the second light energy returned in the form of the second returning energy beam 368R. The two paths provide one to transmit the second assembly illumination beam 368B into the guide proximal end 322P of the light guide 322A and one to collect the second light energy of the second returning energy beam 368R returning from the guide distal end 322D.

At least a portion of the second returning energy beam 368R is then redirected by the first beamsplitter 374 through the second optical element 380 and onto the photodetector 382. The second optical element 380 focuses the collimated second returning energy beam 368R, forming an image of the end face of the light guide 322A onto the photodetector 382, thereby coupling light emitted from the guide proximal end 322P of the light guide 322A.

The photodetector 382 then generates a signal based on the strength of the light at the specific wavelength. In some embodiments, the photodetector 382 generates a signal that is based on the second light energy or second returning energy beam 368R reflected and/or scattered back from the vessel wall 308A that has been received by the distal light receiver 322R at or near the guide distal end 322D of the light guide 322A and that has been collected by the photodetector 382.

The signal from the photodetector 382 is directed toward the amplifier 384 where the signal from the photodetector 382 is amplified before being sent to the control electronics 386 for processing and analysis. Alternatively, in other embodiments, the contact detector assembly 342 can be designed without the amplifier 384. In such alternative embodiments, the signal from the photodetector 382 can be sent to the control electronics 386 for processing and analysis. In either embodiment, it can be stated that the signal from the photodetector 382 is being used by the control electronics 386 to determine the contact condition between the balloon wall 330 and the vessel wall 308A (illustrated in FIG. 3A).

Returning again to FIG. 2, at step 207, the signals generated during analysis of the first returning energy beam and the second returning energy beam are compared to determine the quality of contact that exists between the balloon wall and the vessel wall.

In particular, as described in this operational mode, the contact detector assembly 342 could pulse or modulate the two assembly illumination sources 366, 368 in time to enable the photodetector 382 and/or the control electronics 386 to distinguish them through temporal analysis or gating. More specifically, the control electronics 386 would analyze a ratio of the returned signals. This method provides a means of auto-calibration for the source intensity, coupling factors, transmission factors and variability in the geometry and optics of the region being analyzed.

Thus, in certain implementations, the method used in this embodiment is configured to pulse the two assembly illumination sources 366, 368 at closely spaced time intervals. The control electronics 386 would send a short pulse to the first source driver 366A for the first assembly illumination source 366 at a first wavelength and then read the signal from the amplified photodetector. That would provide the absorption for the first wavelength. The control electronics 386 would then send a short pulse to the second source driver 368A for the second assembly illumination source 368 at a second wavelength and then read the signal from the amplified photodetector. That would provide the absorption for the second wavelength. The pulses could have very short duration, a few μs to tens of μs and be spaced close in time, down their FWHM as the pulse shape approaches rectangular. This would allow the contact detector assembly 342 to run continuously with a very high sampling rate. Many modulation and pulse shaping schemes are possible.

It is appreciated, however, that there are other ways to accomplish this spectral multiplexing. For example, in one non-exclusive alternative embodiment, the contact detector assembly 342 could include two separate photodetectors, each having a narrow bandpass filter (BPF). In one embodiment, the beam focused by the imaging lens could be split using a dichroic mirror onto two separate photodetectors. The dichroic mirror would thus create a symmetric multiplexer-demultiplexer (MDM). Both assembly illumination sources 366, 368 could then be operated in continuous wave (CW) mode allowing continuous scanning. The photodetector 382 could be a spectrometer or multiple single detectors with the beam separated between them using dispersive element such as a grating or prism. The illumination and detection sections could be inverted across the beamsplitter, with the detectors and imaging lens on the transmission face and the probe illumination on the reflection face.

As described, two different wavelength sources are used in this embodiment, λ₁ and λ₂. Light from the first assembly illumination source 366 at λ₁ would be attenuated if blood 305 is present in the space between the balloon wall 330 and the vessel wall 308A. Light from the second assembly illumination source 368 at λ₂ would pass through blood 305 and scatter off the vessel wall 308A.

When blood 305 is present as in the second (bad or suboptimal) condition 390B, λ₁ would be attenuated and very little would return to the photodetector 382. The signal, S(λ₁) would be very small. However, λ₂ would pass through blood 305, scatter back through and return to the photodetector 382. The signal, S(λ₂) would be greater. Some losses will occur due to scattering by blood corpuscles. Conversely, when little or no blood 305 is present as in the first (good or optimal) condition 390A, both wavelengths would be scattered back and return to the photodetector 382 making S(λ₁) comparable to S(λ₂).

The ratio of the two signals would then provide an indicator of the presence or absence of blood 305 and how thick the layer is. Since both assembly illumination beams 366B, 368B are transmitted through the same optical path out to the guide distal end 322D and both detected sources are collected and transmitted through the same path including the collection optics, this ratiometric approach cancels all these factors out. This makes the system self-calibrating.

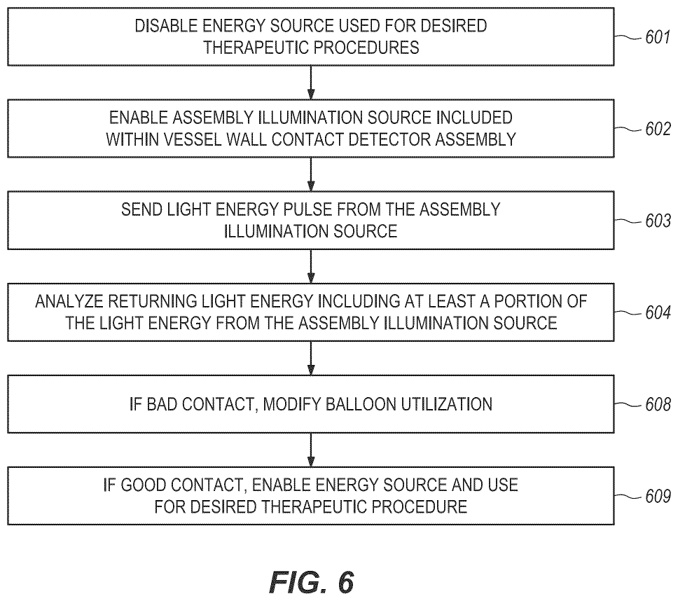

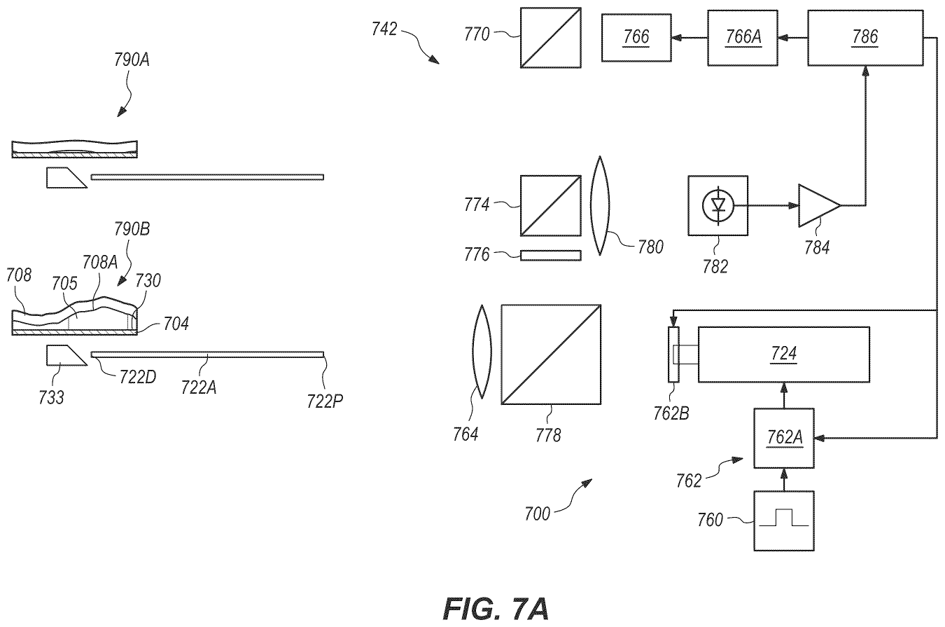

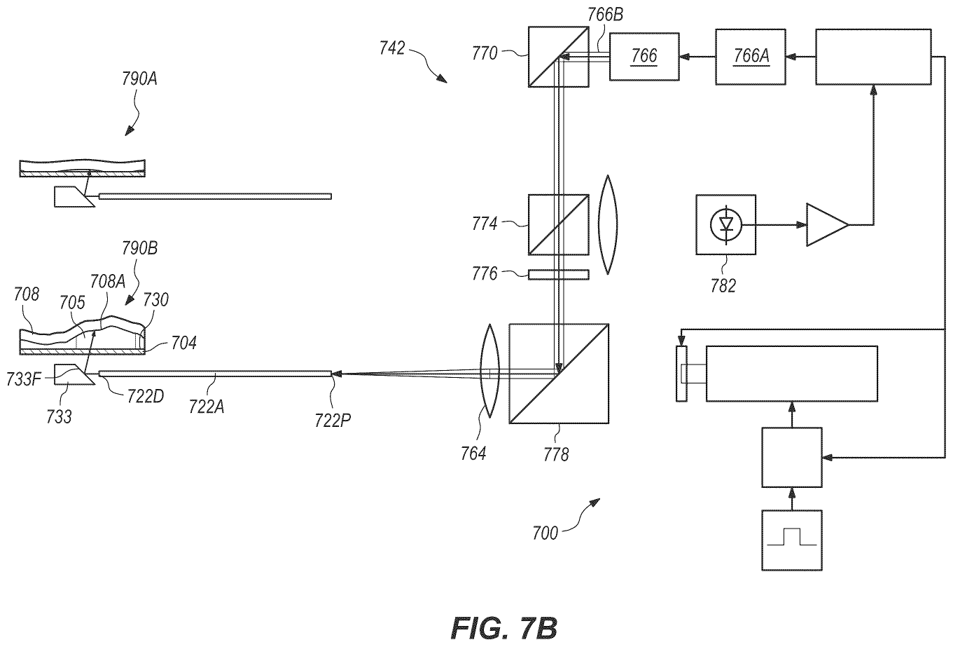

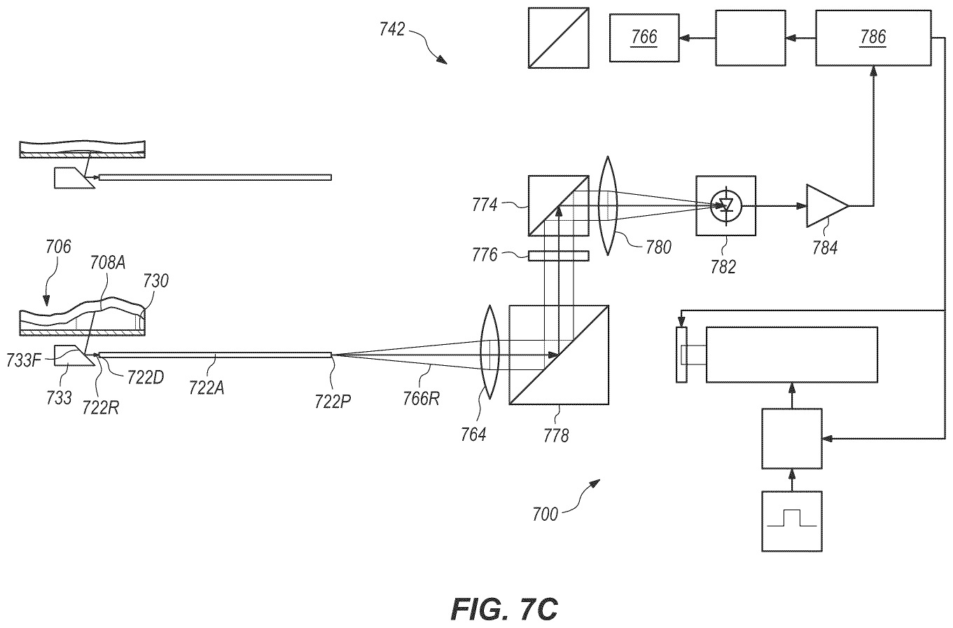

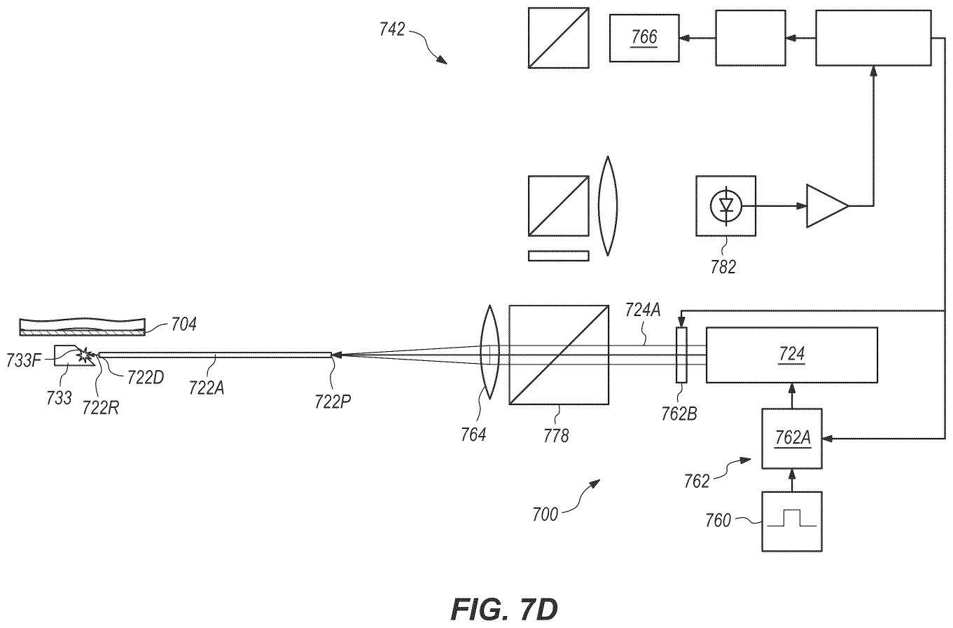

Alternatively, as described herein below in relation to FIGS. 6 and 7A-7D, it would be possible to implement the invention using a single wavelength source. For example, just wavelength λ₁ that is highly transmitted by blood. As the thickness of the layer of blood increases, attenuation of the light passing through the layer and returning from the vessel wall will increase. Less light will return and be collected, and the layer thickness increases S(λ₁), will vary proportionally. This approach requires calibration to null out the effects of coupling and transmission through each part of the optical path. This could work as a go-no go approach to indicate the presence of blood causing a gap between the balloon wall and the vessel wall.

In other embodiments, the assembly illumination source(s) can comprise narrow bands at a plurality of wavelengths. The photodetector 382 could have multiple discrete photodetectors and NBP filters, as described before, or it could be a spectrophotometer. The detection scheme would then involve a more complex calculation based on multiple wavelengths that could account for transmission losses, coupling at the plasma generator 333, scattering in blood and many other physical phenomena that would impact the accuracy of this photodetector 382. In the broadest embodiment, the source would be broadband, such as a high intensity white LED and the photodetector 382 would be a spectrometer. A supercontinuum white light laser (WLL) could work in this application and many other sources are possible. It would be possible with this approach to capture the spectrums of the returned light and decompose this into spectra for blood and tissue separately using eigen decomposition methods or the like. That would provide a high accuracy indicator of the presence of blood or other fluid in the space between the balloon wall 330 and the vessel wall 308A. The two-wavelength approach is the limiting case of this.

At step 208, if the comparison undertaken in step 207 determines that bad, or suboptimal, contact exists between the balloon wall and the vessel wall, then the present utilization of the balloon is modified. In certain non-exclusive situations, the modification of usage of the balloon can entail inflating the balloon to a different level (adjusting the internal balloon pressure), moving the balloon to a modified position relative to the treatment site, and/or removing the catheter and utilizing a catheter having a balloon that is more properly sized for the present treatment procedure.

At step 209, if the comparison undertaken in step 207 determines that good, or optimal, contact exists between the balloon wall and the vessel wall, then the light source (or other suitable energy source) is enabled and used within the catheter system for the desired therapeutic procedure.

Referring now to FIG. 3F, FIG. 3F is a simplified schematic view illustration of the portion of the embodiment of the catheter system 300 including the embodiment of the vessel wall contact detector assembly 342 illustrated in FIG. 3A, with step 209 within the first operational mode being illustrated.

In particular, as illustrated, the light source 324 has been enabled such that the safety interlock system 362, including the interlock 362A and the shutter 362B, is no longer inhibiting the generation and/or directing of light energy from the light source 324. The pulse generator 360 is configured to trigger the light source 324 such that a pulse of light energy is generated by the light source 324. The light energy from the light source 324 is directed in the form of a source beam 324A toward the second beamsplitter 378, such as the dichroic beamsplitter in one embodiment. Stated in another manner, the second beamsplitter 378 is positioned in the optical path of the source beam 324A between the light source 324 and the guide proximal end 322P of the light guide 322A.

In certain embodiments, the second beamsplitter 378 is configured to pass or transmit light with wavelengths longer than those visible to the photodetector 382 so the source beam 324A is directed toward the guide proximal end 322P of the light guide 322A. Such threshold wavelength can be referred to as the cutoff wavelength. The second beamsplitter 378 is further configured to reflect all light having a wavelength that is shorter than the cutoff wavelength.

As illustrated in this embodiment, the first optical element 364 is positioned between the second beamsplitter 378 and the light guide 322A and is configured to collimate the source beam 324A and focus the source beam 324A down onto the guide proximal end 322P of the light guide 322A, thereby coupling the source beam 324A into the light guide 322A.

In various implementations, the source beam 324A is of such a wavelength, such as within the infrared spectrum, that it is transmitted by the second beamsplitter 378, before passing through the first optical element 364 that focuses the light energy of the source beam 324A onto the guide proximal end 322P of the light guide 322A. The light energy of the source beam 324A is then guided along and/or through the light guide 322A and emitted at the guide distal end 322D into the balloon interior 146 (illustrated in FIG. 1) of the balloon 304. The light energy of the source beam 324A impinges upon and energizes the angled face 333F of the plasma generator 333 and is redirected such that a localized plasma is generated within the catheter fluid 132 (illustrated in FIG. 1) in the balloon interior 146.

In one embodiment, the light guide 322A is a small diameter, multimode optical fiber that is used to direct light transmitted from the light source 324 and/or the assembly illumination sources 366, 368, and the plasma generator 333 contains a beveled backstop that directs light toward the vessel wall 308A. The light guide 322A and beveled backstop (angled face 333F) of the plasma generator 333 serve the dual purpose of delivering high-energy light for plasma generation (as illustrated in FIG. 3F) as well as lower energy light for blood and tissue identification (as illustrated in FIGS. 3B and 3D). The beveled backstop (angled face 333F) of the plasma generator 333 is also used in conjunction with and/or as part of the distal light receiver 322R to collect light reflected and/or scattered back from illuminated regions and couple it back into the light guide 322A for transmission back to the contact detector assembly 342 for detection and analysis (as illustrated in FIGS. 3C and 3E).

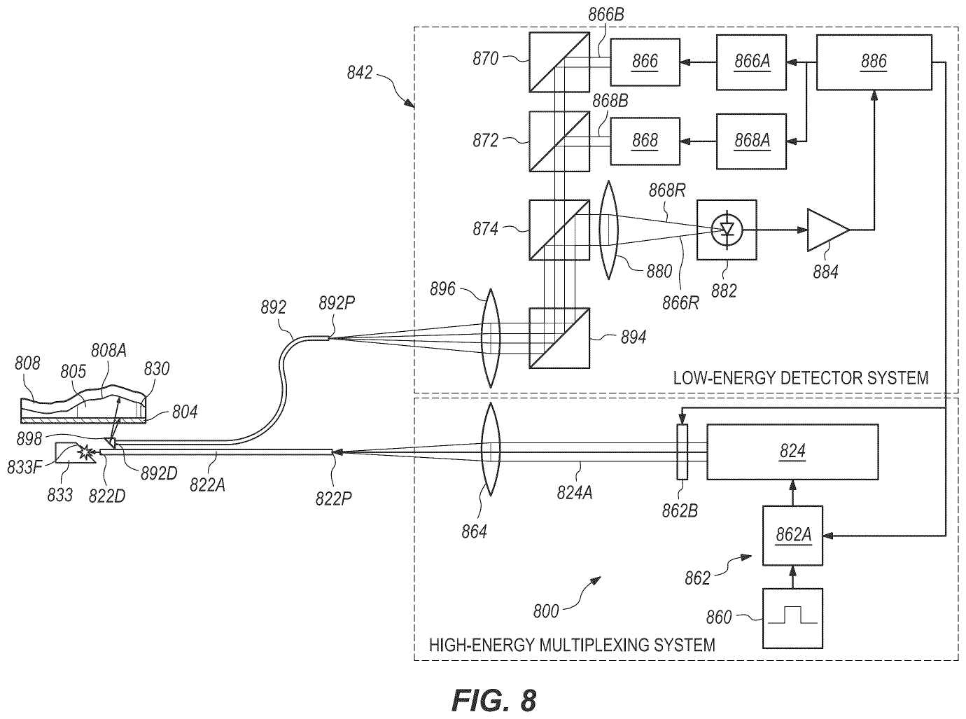

In another embodiment, the contact detector assembly 342 can include a separate illumination beam guide 892 (illustrated in FIG. 8) in the form of an optical fiber that is used for blood and tissue identification, in addition to the light guide 322A in the form of an optical fiber that is used for high-energy transmission and plasma generation. In conjunction with the illumination beam guide 892, in one embodiment, an energy directing element, such as a mirror, could be affixed to a distal end of the illumination beam guide 892 to direct the first light energy from the assembly illumination beams 366B (illustrated in FIG. 3B), 368B (illustrated in FIG. 3D) toward the balloon wall 330 and/or the vessel wall 308A, and to redirect the returning energy beam back into and through the illumination beam guide 892 for subsequent analysis by the contact detector assembly 342. Alternatively, an endface at the distal end of the illumination beam guide 892 could also be beveled to create a side-emitting feature that directs light energy out of the side and similarly collects light scattered into its numerical aperture.

FIG. 3F shows the state of the system when optimal contact between the balloon wall 330 and the vessel wall 308A is achieved. The control electronics 386 have determined the conditions are optimal, turns off the assembly illumination sources 366, 368, and opens the shutter 362B and energizes the light source 324, such as the infrared laser, to generate a plasma to produce the therapeutic acoustic energy.

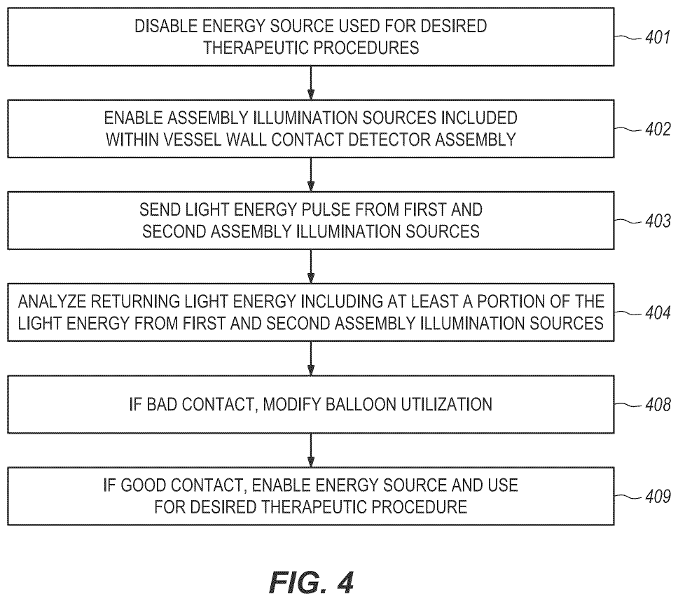

FIG. 4 is a flowchart illustrating a second operational mode for the embodiment of the catheter system including the embodiment of the vessel wall contact detector assembly of FIG. 3A. It is appreciated that any of the steps listed in the flowchart of FIG. 4 can be modified, deleted and/or combined in any suitable manner, and/or the order of steps can be changed, without deviating from the intended breadth and scope of the present invention. One or more steps can also be added to the series of steps specifically illustrated and described within the flowchart without deviating from the intended breadth and scope of the present invention.

At step 401, the light source (or other suitable energy source) that is utilized within the catheter system for desired therapeutic procedures is disabled. The disabling of the light source is undertaken so that light energy from the light source is not directed into and through the light guide until after optimal contact between the balloon wall and the vessel wall has been appropriately established through use of the contact detector assembly.

It is appreciated that the step of disabling the light source can be accomplished in any suitable manner. For example, in certain implementations, the light source can be disabled through use of the safety interlock system, which can employ the safety interlock and/or the shutter, through specific disabling of the pulse generator that is coupled to the light source, and/or through otherwise blocking power from being supplied to the light source.

At step 402, the first assembly illumination source and the second illumination source included within the contact detector assembly are enabled.

At step 403, a first light energy pulse from the first assembly illumination source is generated in the form of a first assembly illumination beam, and a second light energy pulse from the second assembly illumination source is generated in the form of a second assembly illumination beam. As described herein, in the second operational mode as illustrated in FIG. 4, the first assembly illumination beam and the second assembly illumination beam are generated substantially simultaneously, and then are subsequently combined and transmitted together to the light guide. The light guide then guides the combined beam into the balloon interior, from where the light energy of the combined beam is redirected toward the balloon wall of the balloon and/or toward the vessel wall of the blood vessel.

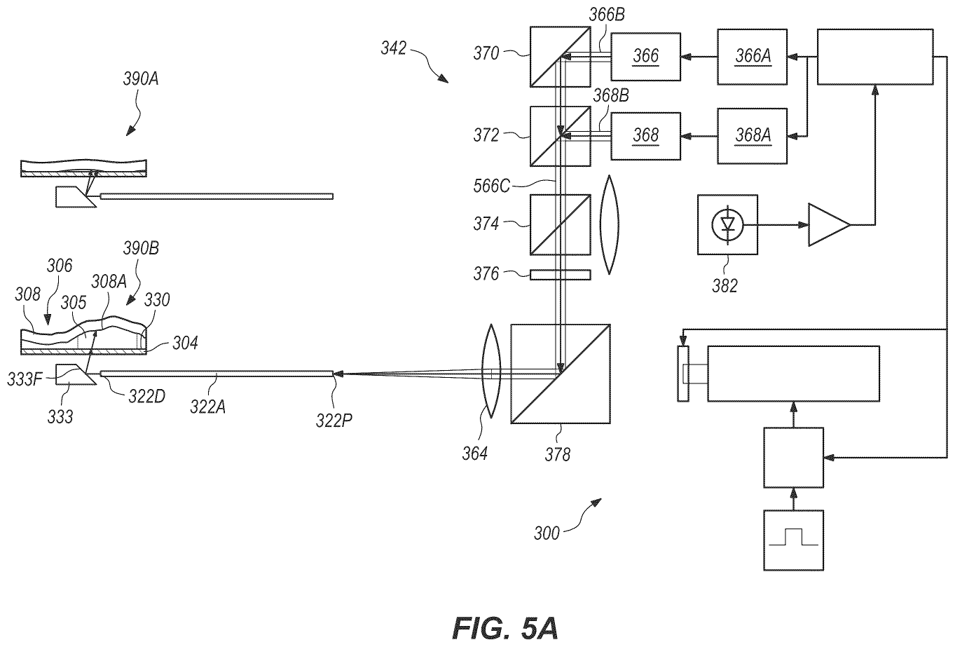

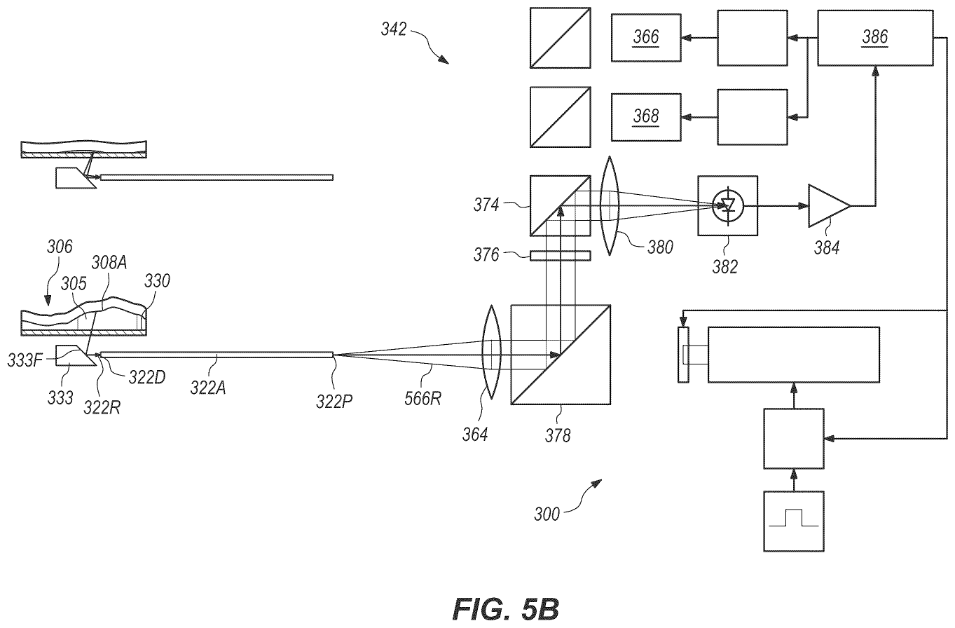

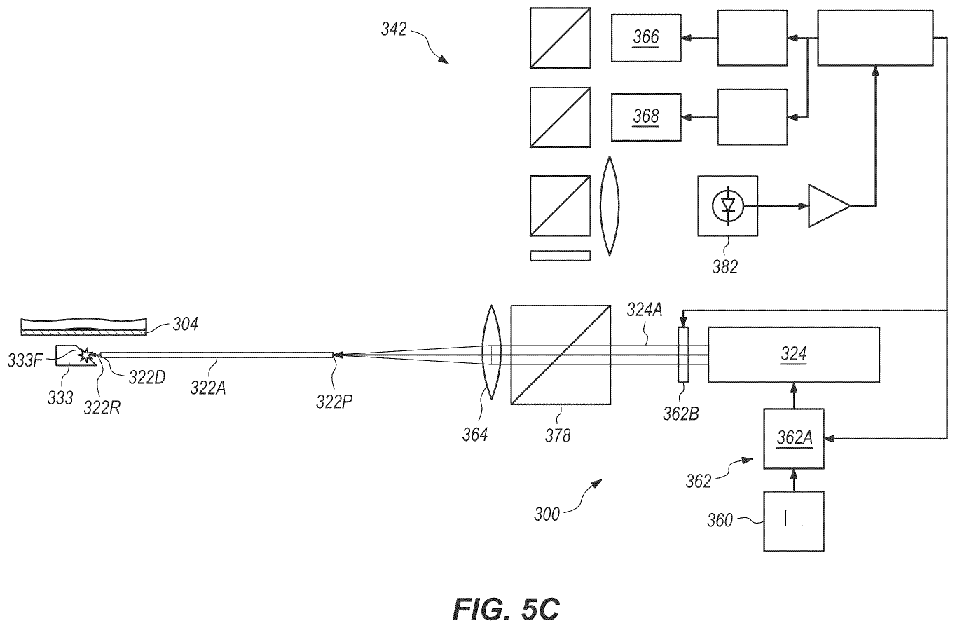

Referring now to FIG. 5A, FIG. 5A is a simplified schematic view illustration of the portion of the embodiment of the catheter system 300 including the embodiment of the vessel wall contact detector assembly 342 of FIG. 3A, with step 403 within the second operational mode being illustrated.

In particular, as illustrated, the first source driver 366A activates the first assembly illumination source 366 to generate a pulse of first light energy in the form of a first assembly illumination beam 366B. Substantially simultaneously, the second source driver 368A activates the second assembly illumination source 368 to generate a pulse of first light energy in the form of a second assembly illumination beam 368B.

The desired wavelength of the first assembly illumination beam 366B and the second assembly illumination beam 368B can be varied. In particular, the wavelength range useful for blood and vessel wall detection spans the full visible range down into near IR. It is bounded in this approach at short wavelengths by transmission limits for the optical fiber towards the UV, which is around 250 nm for IR-fused silica. It is limited at long wavelengths by the absorption spectrum of biological materials and the bandgap of solid-state photodetectors which is 1.1 μm for silicon. Use of more esoteric materials and detectors could extend this range. For example, UV-fused silica could be usable down to 180 nm, and InGaAs photodiodes can detect light out to 1.68 μm. The absorption spectrum for biological materials involved ultimately become limiting factors. As mentioned earlier, it is preferable to use wavelengths at the isosbestic points of Hemoglobin, though this is not essential to the functionality of the method. An isosbestic point is a specific wavelength at which the total absorbance of a sample does not change during a chemical reaction or a physical change of the sample. In this case, the change from deO₂Hb to its oxygenated form, O₂Hb. There are eleven isosbestic points for deO₂Hb and O₂Hb over the 200-900 nm wavelength range: 255, 350, 390, 422, 452, 500, 529, 545, 570, 584, and 805 nm.

In certain non-exclusive embodiments, the first assembly illumination beam 366B can be at a wavelength that is generally strongly reflected when it comes into contact with blood 305, such as approximately 550 nm and 600 nm. Additionally, in certain non-exclusive embodiments, the second assembly illumination beam 368B can be at a wavelength that is generally transmitted by the blood 305 and then strongly reflected when it comes into contact with vessel wall 308A of the blood vessel 308, such as approximately 640 nm and 750 nm. Alternatively, the first assembly illumination beam 366B and/or the second assembly illumination beam 368B can be at another suitable wavelength.

The first assembly illumination beam 366B is initially directed toward and is redirected by the first redirector 370, such as a mirror in one non-exclusive embodiment, which in certain implementations can redirect the first assembly illumination beam 366B by approximately ninety degrees. The first assembly illumination beam 366B is then directed toward the second redirector 372, such as a dichroic mirror that transmits light at certain wavelengths and redirects light at other wavelengths. At substantially the same time, the second assembly illumination beam 368B is initially directed toward the second redirector 372.

Due to the different wavelengths of the first assembly illumination beam 366B and the second assembly illumination beam 368B, and the specific design of the second redirector 372, the first assembly illumination beam 366B is transmitted by the second redirector 372 and the second assembly illumination beam 368B is redirected by the second redirector 372. Because the assembly illumination beams 366B, 368B have been generated and directed substantially simultaneously, the second redirector 372 functions to combine the assembly illumination beams 366B, 368B into a combined illumination beam 566C that is then directed toward and impinges upon the first beamsplitter 374.

At least a portion of the combined illumination beam 566C is transmitted by the first beamsplitter 374, which can have any suitable design such as described above. The portion of the combined illumination beam 566C that has been transmitted by the first beamsplitter 374 then passes through the filter 376 before impinging upon the second beamsplitter 378, which can be a dichroic beamsplitter in certain embodiments that transmits light at certain wavelengths and redirects light at other wavelengths. In certain embodiments, the second beamsplitter 378 is configured to pass or transmit light with wavelengths longer than those visible to the photodetector 382. Such threshold wavelength can be referred to as the cutoff wavelength. The second beamsplitter 378 is further configured to reflect all light having a wavelength that is shorter than the cutoff wavelength. It is appreciated that the combined illumination beam 566C will be at such a wavelength that it will be reflected substantially in its entirety by the second beamsplitter 378.

As shown, the remaining portion of the combined illumination beam 566C is redirected by the second beamsplitter 378 before passing through the first optical element 364 which collimates the combined illumination beam 566C and focuses the first light energy from the portion of the combined illumination beam 566C onto the guide proximal end 322P of the light guide 322A. The first light energy from the portion of the combined illumination beam 566C is guided through the light guide 322A and is emitted at the guide distal end 322D of the light guide 322A before it impinges upon the plasma generator 333. In certain embodiments, the angled face 333F of the plasma generator 333 redirects the first light energy of the combined illumination beam 566C toward the balloon wall 330 of the balloon 304 and/or the vessel wall 308A of the blood vessel 308 at the treatment site 306. Thus, the angled face 333F of the plasma generator 333 acts like a single surface mirror. The beam spot of the combined illumination beam 566C at the surface of the angled face 333F is narrow due to the low numerical aperture of the light guide 322A when immersed in the catheter fluid 132 (illustrated in FIG. 1). Due to the different wavelengths included within the combined illumination beam 566C, the redirected combined illumination beam 566C will in turn form highly concentrated spots on the balloon wall 330 and nearby tissue, such as the vessel wall 308A of the blood vessel 308.

As described in greater detail herein below, one intense spot from the portion of the combined illumination beam 566C that originated as the first assembly illumination beam 366B at the first wavelength will scatter from the blood 305, and one intense spot from the portion of the combined illumination beam 566C that originated as the second assembly illumination beam 368B at the second wavelength will scatter from the vessel wall 308A. The portions of the combined illumination beam 566C scattered back from the blood 305 and the vessel wall 308A are then directed back toward the guide distal end 322D of the light guide 322A by the angled face 333F where they will be collected within the numerical aperture of the guide distal end 322D of the light guide 322A.

As shown in FIG. 5A, the first light energy from the portion of the combined illumination beam 566C originating from the first assembly illumination beam 366B is generally reflected when it contacts the blood 305 whether the balloon 304 is in a good contact condition 390A or in a bad contact condition 390B; and the first light energy from the portion of the combined illumination beam 566C originating from the second assembly illumination beam 368B passes through the blood 305 and then is generally reflected when it contacts the vessel wall 308A whether the balloon 304 is in a good contact condition 390A or in a bad contact condition 390B.

Thus, as described, the contact detector assembly 342 includes two collimated assembly illumination sources 366, 368, each having a specific wavelength. These sources could be diode lasers, high intensity LEDs with optics to collimate the emitted beam, or other solid-state light emitting sources. This embodiment of the invention uses two distinct wavelengths, one with a high absorption coefficient for blood and one with a low absorption coefficient. Ideally, these will both be at isosbestic points for the absorption spectrums of O₂Hb and deO₂Hb so that the relative oxygenation of the blood would not affect the relative absorption or transmission of the light. The assembly illumination beams 366B, 368B from the two assembly illumination sources 366, 368 in this embodiment are combined into a single combined illumination beam 566C using the second redirector 372, such as a dichroic mirror. The single bi-spectral combined illumination beam 566C is reflected off the high-energy second beamsplitter 378 and coupled in the guide proximal end 322P of the light guide 322A. The light is emitted from the guide distal end 322D of the light guide 322A and is directed toward the balloon wall 330 and/or the vessel wall 308A by the angled face 333F of the plasma generator 333.

Returning again to FIG. 4, at step 404, a portion of the first light energy from the combined illumination beam is reflected and/or scattered back from the blood, and another portion of the first light energy from the combined illumination beam is reflected and/or scattered back from the vessel wall. Such portions of light energy are then guided back through the light guide, and are subsequently analyzed by the contact detector assembly.

Referring now to FIG. 5B, FIG. 5B is a simplified schematic view illustration of the portion of the embodiment of the catheter system 300 including the embodiment of the vessel wall contact detector assembly 342 of FIG. 3A, with step 404 within the second operational mode being illustrated.

In particular, as illustrated, a portion of the first light energy from the combined illumination beam 566C (illustrated in FIG. 5A) originating from the first assembly illumination source 366 that is incident on the balloon wall 330 is reflected and/or scattered back from the blood 305, and a portion of the first light energy from the combined illumination beam 566C originating from the second assembly illumination source 368 that is incident on the balloon wall 330 is reflected and/or scattered back from the vessel wall 308A. Such portions of first light energy from the combined illumination beam 566C are reflected and/or scattered back as second light energy and/or a combined returning energy beam 566R that is received by the distal light receiver 322R that couples the second light energy and/or the combined returning energy beam 566R into the guide distal end 322D of the light guide 322A. In certain embodiments, the angled face 333F of the plasma generator 333 can collect the second light energy reflected and/or scattered back from the blood 305 and the vessel wall 308A, and redirect the second light energy back into the guide distal end 322D of the light guide 322A. Thus, the angled face 333F of the plasma generator 333 acts like a single surface mirror and can function as at least a portion of the distal light receiver 322R.

The second light energy and/or the combined returning energy beam 566R is then emitted from the guide proximal end 322P from where it is directed through the first optical element 364, which collimates the combined returning energy beam 566R. The collimated combined returning energy beam 566R is then directed to the second beamsplitter 378, such as the dichroic beamsplitter.

Due to the specific wavelengths included within the combined returning energy beam 566R, at least a portion of the combined returning energy beam 566R is redirected by the second beamsplitter 378 and then is directed through the filter 376 before it impinges on the first beamsplitter 374. Stated in another manner, the first beamsplitter 374 is positioned in the optical path of the combined returning energy beam 566R reflected from the second beamsplitter 378. This creates a two-way system for probing the treatment site 306 through the light guide 322A and detection of conditions there by the second light energy returned in the form of the combined returning energy beam 566R. The two paths provide one to transmit the combined illumination beam 566C into the guide proximal end 322P of the light guide 322A and one to collect the second light energy of the combined returning energy beam 566R returning from the guide distal end 322D.

At least a portion of the combined returning energy beam 566R is then redirected by the first beamsplitter 374 through the second optical element 380 and onto the photodetector 382. The second optical element 380 focuses the collimated combined returning energy beam 566R, forming an image of the end face of the light guide 322A onto the photodetector 382, thereby coupling light emitted from the guide proximal end 322P of the light guide 322A. In certain non-exclusive alternative embodiments, the photodetector 382 can be a photodiode, an area sensor such as a CCD or CMOS camera, or a spectrophotometer. In suitable arrangements the chain of optical elements 364, 380 can create a high-resolution image of the end face of the light guide 322A on an image sensor.

The photodetector 382 then generates a signal based on the strength of the light at each of the two specific wavelengths. In some embodiments, the photodetector 382 generates (i) a first signal that is based on the second light energy of the combined returning energy beam 566R reflected and/or scattered back from the blood 305 that has been received by the distal light receiver 322R at or near the guide distal end 322D of the light guide 322A and that has been collected by the photodetector 382; and (ii) a second signal that is based on the second light energy of the combined returning energy beam 566R reflected and/or scattered back from the vessel wall 308A that has been received by the distal light receiver 322R at or near the guide distal end 322D of the light guide 322A and that has been collected by the photodetector 382. Stated in another manner, the photodetector 382 generates (i) a first signal that is based on the second light energy of the combined returning energy beam 566R that is at the first wavelength; and (ii) a second signal that is based on the second light energy of the combined returning energy beam 566R that is at the second wavelength.

The signals from the photodetector 382 are directed toward the amplifier 384 where the signals from the photodetector 382 are amplified before being sent to the control electronics 386 for processing and analysis. Alternatively, in other embodiments, the contact detector assembly 342 can be designed without the amplifier 384. In such alternative embodiments, the signals from the photodetector 382 can be sent to the control electronics 386 for processing and analysis. In either embodiment, it can be stated that the signals from the photodetector 382 are being used by the control electronics 386 to determine the contact condition between the balloon wall 330 and the vessel wall 308A.

With the two assembly illumination sources 366, 368 combined into one beam, both wavelengths would be present at the single photodetector 382. It is necessary to discriminate between different wavelengths in a multispectral approach to obtain separate spectral signals for ratiometric analysis. The method used in this embodiment can use a dichroic beamsplitter or other optical means of separating the two returned sources such as a grating or spectroscope. The photodetector 382 would then generate the separate first signal and second signal that are representative of the absorption for the first wavelength and the second wavelength, respectively.

As noted, and similar to the previous embodiments, two different wavelength sources are used in this embodiment, λ₁ and λ₂. Light from the first assembly illumination source 366 at λ₁ would be attenuated if blood 305 is present in the space between the balloon wall 330 and the vessel wall 308A. Light from the second assembly illumination source 368 at λ₂ would pass through blood 305 and scatter off the vessel wall 308A.

When blood 305 is present as in the second (bad or suboptimal) condition 390B, λ₁ would be attenuated and very little would return to the photodetector 382. The signal, S(λ₁) would be very small. However, λ₂ would pass through blood 305, scatter back through and return to the photodetector 382. The signal, S(λ₂) would be greater. Some losses will occur due to scattering by blood corpuscles. Conversely, when little or no blood 305 is present as in the first (good or optimal) condition, both wavelengths would be scattered back and return to the photodetector 382 making S(λ₁) comparable to S(λ₂).

The ratio of the two signals would then provide an indicator of the presence or absence of blood 305 and how thick the layer is. Since both assembly illumination beams 366B, 368B are transmitted through the same optical path out to the guide distal end 322D and both detected sources are collected and transmitted through the same path including the collection optics, this ratiometric approach cancels all these factors out. This makes the system self-calibrating.

Returning again to FIG. 4, at step 408, if the analysis undertaken in step 404 determines that bad, or suboptimal, contact exists between the balloon wall and the vessel wall, then the present utilization of the balloon is modified. In certain non-exclusive situations, the modification of usage of the balloon can entail inflating the balloon to a different level (adjusting the internal balloon pressure), moving the balloon to a modified position relative to the treatment site, and/or removing the catheter and utilizing a catheter having a balloon that is more properly sized for the present treatment procedure.

At step 409, if the analysis undertaken in step 404 determines that good, or optimal, contact exists between the balloon wall and the vessel wall, then the light source (or other suitable energy source) is enabled and used within the catheter system for the desired therapeutic procedure.

Referring now to FIG. 5C, FIG. 5C is a simplified schematic view illustration of the portion of the embodiment of the catheter system 300 including the embodiment of the vessel wall contact detector assembly 342 illustrated in FIG. 3A, with step 409 within the second operational mode being illustrated.

In particular, as illustrated, the light source 324 has been enabled such that the safety interlock system 362, including the interlock 362A and the shutter 362B, is no longer inhibiting the generation and/or directing of light energy from the light source 324. The pulse generator 360 is configured to trigger the light source 324 such that a pulse of light energy is generated by the light source 324. The light energy from the light source 324 is directed in the form of a source beam 324A toward the second beamsplitter 378, such as the dichroic beamsplitter in one embodiment. Stated in another manner, the second beamsplitter 378 is positioned in the optical path of the source beam 324A between the light source 324 and the guide proximal end 322P of the light guide 322A.

In certain embodiments, the second beamsplitter 378 is configured to pass or transmit light with wavelengths longer than those visible to the photodetector 382 so the source beam 324A is directed toward the guide proximal end 322P of the light guide 322A. Such threshold wavelength can be referred to as the cutoff wavelength. The second beamsplitter 378 is further configured to reflect all light having a wavelength that is shorter than the cutoff wavelength.

As illustrated in this embodiment, the first optical element 364 is positioned between the second beamsplitter 378 and the light guide 322A and is configured to collimate the source beam 324A and focus the source beam 324A down onto the guide proximal end 322P of the light guide 322A, thereby coupling the source beam 324A into the light guide 322A.

In various implementations, the source beam 324A is of such a wavelength, such as within the infrared spectrum, that it is transmitted by the second beamsplitter 378, before passing through the first optical element 364 that focuses the light energy of the source beam 324A onto the guide proximal end 322P of the light guide 322A. The light energy of the source beam 324A is then guided along and/or through the light guide 322A and emitted at the guide distal end 322D into the balloon interior 146 (illustrated in FIG. 1) of the balloon 304. The light energy of the source beam 324A impinges upon and energizes the angled face 333F of the plasma generator 333 and is redirected such that a localized plasma is generated within the catheter fluid 132 (illustrated in FIG. 1) in the balloon interior 146.

In one embodiment, the light guide 322A is a small diameter, multimode optical fiber that is used to direct light transmitted from the light source 324 and/or the assembly illumination sources 366, 368, and the plasma generator 333 contains a beveled backstop that directs light toward the vessel wall 308A. The light guide 322A and beveled backstop (angled face 333F) of the plasma generator 333 serve the dual purpose of delivering high-energy light for plasma generation (as illustrated in FIG. 5C) as well as lower energy light for blood and tissue identification (as illustrated in FIG. 5A). The beveled backstop (angled face 333F) of the plasma generator 333 is also used in conjunction with and/or as part of the distal light receiver 322R to collect light reflected and/or scattered back from illuminated regions and couple it back into the light guide 322A for transmission back to the contact detector assembly 342 for detection and analysis (as illustrated in FIG. 5B).

FIG. 6 is a flowchart illustrating an operational mode for another embodiment of the catheter system including another embodiment of the vessel wall contact detector assembly. It is appreciated that any of the steps listed in the flowchart of FIG. 6 can be modified, deleted and/or combined in any suitable manner, and/or the order of steps can be changed, without deviating from the intended breadth and scope of the present invention. One or more steps can also be added to the series of steps specifically illustrated and described within the flowchart without deviating from the intended breadth and scope of the present invention.

Referring now to FIG. 7A, FIG. 7A is a simplified schematic view illustration of a portion of the embodiment of the catheter system 700 including the embodiment of the vessel wall contact detector assembly 742 usable within the operational mode as described in relation to FIG. 6.

The design of the catheter system 700 is substantially similar to the embodiments illustrated and described herein above. It is appreciated that various components of the catheter system 700, such as are shown in FIG. 1, are not illustrated in FIG. 7A for purposes of clarity and ease of illustration. However, it is appreciated that the catheter system 700 will likely include most, if not all, of such components.

As shown in FIG. 7A, the catheter system 700 again includes one or more of a light source 724 (such as a pulsed infrared laser source in one non-exclusive embodiment, or another suitable energy source), a pulse generator 760 that is coupled to the light source 724, a safety shutdown system 762 including a safety interlock 762A and a shutter 762B, a first optical element 764 (such as a coupling lens in one non-exclusive embodiment), a light guide 722A (such as an optical fiber in one non-exclusive embodiment, or another suitable energy guide), a plasma generator 733, a balloon 704 including a balloon wall 730, and the contact detector assembly 742.

However, the contact detector assembly 742 has a slightly different design than the previous embodiments. For example, as further shown in FIG. 7A, the contact detector assembly 742 can again include one or more of a first beamsplitter 774, a filter 776, a second beamsplitter 778, a second optical element 780 (such as an imaging lens in one non-exclusive embodiment), a photodetector 782, an amplifier 784, and control electronics 786, which can include one or more processors or circuits. However, in this embodiment, the contact detector assembly 742 only includes one assembly illumination source 766, one source driver 766A, and one redirector 770. Alternatively, in other embodiments, the catheter system 700 and/or the contact detector assembly 742 can include more components or fewer components than those specifically noted above. Still alternatively, in still other embodiments, the various components of the catheter system 700 and/or the contact detector assembly 742 can be positioned in a different manner than what is specifically illustrated in FIG. 7A.

FIG. 7A also shows that, during use of the catheter system 700 and/or the contact detector assembly 742, the balloon wall 730 of the balloon 704 can be positioned (i) in a first condition 790A—in good and/or optimal contact with the vessel wall 708A of the blood vessel 708, such as with little or no blood 705 positioned between the balloon wall 730 and the vessel wall 708A, and/or (ii) in a second condition 790B—in bad and/or suboptimal contact with the vessel wall 708A of the blood vessel 708, such as with a relatively thick layer of blood 705 positioned between the balloon wall 730 and the vessel wall 708A. In various embodiments, the contact detector assembly 742 is again configured to establish whether the balloon wall 730 of the balloon 704 is positioned relative to the vessel wall 708A of the blood vessel 708 in the first condition 790A (with good or optimal contact between the balloon wall 730 and the vessel wall 708A) or in the second condition 790B (with bad or suboptimal contact between the balloon wall 730 and the vessel wall 708A).

In this embodiment, the contact detector assembly 742 includes just one assembly illumination source 766 at a single specific wavelength. In particular, with this embodiment, the simplest detection method is to illuminate using narrow band light with a center wavelength that is highly transmitted by the blood 705 but is reflected from the vessel wall 708A. This ideally would be at an isosbestic points for oxy-hemoglobin (O₂Hb) and deoxy-hemoglobin (deO₂Hb) spectrum. This would reduce the impact of blood oxygenation on the signal. As illustrated, and as described in greater detail herein below, the assembly illumination source 766 is coupled to the guide proximal end 722P of the light guide 722A and the light emitted from the guide distal end 722D is directed toward the vessel wall 708A. If blood 705 is present in the space between the balloon wall 730 and the vessel wall 708A, then most of it will be absorbed and very little will be scattered back, collected at the guide distal end 722D of the light guide 722A, and returned to the photodetector 782. If no blood 705 is present, then the light will be transmitted to the vessel wall 708A and scattered back to the photodetector 782. With this method, the system would be looking for a strong return signal to indicate good contact between the balloon wall 730 and the vessel wall 708A; whereas a small or absent signal would indicate poor contact. This is an absolute detection method that would depend on multiple intensity, coupling and transmission factors, likely requiring some means of calibration.

Returning to FIG. 6, at step 601, the light source (or other suitable energy source) that is utilized within the catheter system for desired therapeutic procedures is disabled. The disabling of the light source is undertaken so that light energy from the light source is not directed into and through the light guide until after optimal contact between the balloon wall and the vessel wall has been appropriately established through use of the contact detector assembly.

It is appreciated that the step of disabling the light source can be accomplished in any suitable manner. For example, in certain implementations, the light source can be disabled through use of the safety interlock system, which can employ the safety interlock and/or the shutter, through specific disabling of the pulse generator that is coupled to the light source, and/or through otherwise blocking power from being supplied to the light source.

At step 602, the assembly illumination source included within the contact detector assembly is enabled.

At step 603, a first light energy pulse from the assembly illumination source is generated in the form of an assembly illumination beam, and is transmitted toward and through the light guide into the balloon interior. The first light energy of the assembly illumination beam can then be redirected, such as by the plasma generator in one embodiment, toward the balloon wall of the balloon and/or toward the vessel wall of the blood vessel.

Referring now to FIG. 7B, FIG. 7B is a simplified schematic view illustration of the portion of the embodiment of the catheter system 700 including the embodiment of the vessel wall contact detector assembly 742 illustrated in FIG. 7A, with step 603 within the operational mode being illustrated.

In particular, as illustrated, the source driver 766A activates the assembly illumination source 766 to generate a pulse of first light energy in the form of an assembly illumination beam 766B that is directed from the assembly illumination source 766 toward the light guide 722A. The desired wavelength of the assembly illumination beam 766B can be varied. In particular, the wavelength range useful for blood and vessel wall detection spans the full visible range down into near IR. It is bounded in this approach at short wavelengths by transmission limits for the optical fiber towards the UV, which is around 250 nm for IR-fused silica. It is limited at long wavelengths by the absorption spectrum of biological materials and the bandgap of solid-state photodetectors which is 1.1 μm for silicon. Use of more esoteric materials and detectors could extend this range. For example, UV-fused silica could be usable down to 180 nm, and InGaAs photodiodes can detect light out to 1.68 μm. The absorption spectrum for biological materials involved ultimately become limiting factors. As mentioned earlier, it is preferable to use wavelengths at the isosbestic points of Hemoglobin, though this is not essential to the functionality of the method. An isosbestic point is a specific wavelength at which the total absorbance of a sample does not change during a chemical reaction or a physical change of the sample. In this case, the change from deO₂Hb to its oxygenated form, O₂Hb. There are eleven isosbestic points for deO₂Hb and O₂Hb over the 200-900 nm wavelength range: 255, 350, 390, 422, 452, 500, 529, 545, 570, 584, and 805 nm.

In certain non-exclusive embodiments, the assembly illumination beam 766B can be at a wavelength that passes through and/or is transmitted by the blood 705 and then is generally reflected when it comes into contact with vessel wall 708A, such as approximately 640 nm and 750 nm. Alternatively, the assembly illumination beam 766B can be at another suitable wavelength.

The assembly illumination beam 766B is initially directed toward and is redirected by the redirector 770, such as a mirror in one non-exclusive embodiment, which in certain implementations can redirect the assembly illumination beam 766B by approximately ninety degrees. The assembly illumination beam 766B is then directed toward and impinges upon the first beamsplitter 774.

At least a portion of the assembly illumination beam 766B is transmitted by the first beamsplitter 774, which can be a non-polarizing variable beamsplitter such as a 50/50 R/T in certain non-exclusive embodiments. In one embodiment, the assembly illumination source 766 is a diode laser with high intensity so enabling a low transmission beamsplitter to be used, such as 90/10; this would allow a much greater return signal for the contact detector assembly 742. It would also be possible to use a polarizing beamsplitter and polarize the assembly illumination beam 766B from the assembly illumination source 766 for 100% transmission.

The portion of the assembly illumination beam 766B that has been transmitted by the first beamsplitter 774 then passes through the filter 776, such as a short pass filter in one non-exclusive embodiment. As such, the illumination and detection systems of the contact detector assembly 742 are isolated from the high-energy light source 724 using the filter 776. This would eliminate any back-reflected light during pulsed plasma operation, improving SNR for the photodetector 782 to enable the contact detector assembly 742 to operate when the catheter system 700 is in therapeutic mode.

After passing through the filter 776, the portion of the assembly illumination beam 766B then impinges upon the second beamsplitter 778, which can be a dichroic beamsplitter in certain embodiments that transmits light at certain wavelengths and redirects light at other wavelengths. In certain embodiments, the second beamsplitter 778 is configured to pass or transmit light with wavelengths longer than those visible to the photodetector 782. Such threshold wavelength can be referred to as the cutoff wavelength. The second beamsplitter 778 is further configured to reflect all light having a wavelength that is shorter than the cutoff wavelength. It is appreciated that the assembly illumination beam 766B will be at such a wavelength that it will be reflected substantially in its entirety by the second beamsplitter 778.

As shown, the remaining portion of the assembly illumination beam 766B is redirected by the second beamsplitter 778 before passing through the first optical element 764 which collimates the assembly illumination beam 766B and focuses the first light energy from the portion of the assembly illumination beam 766B onto a guide proximal end 722P of the light guide 722A. The first light energy from the portion of the assembly illumination beam 766B is guided through the light guide 722A and is emitted at a guide distal end 722D of the light guide 722A before it impinges upon the plasma generator 733. In certain embodiments, the plasma generator 733 can be provided in the form of a backstop-type structure with an angled face 733F that redirects the first light energy toward the balloon wall 730 of the balloon 704 and/or the vessel wall 708A of the blood vessel 708 at the treatment site 706. Thus, the angled face 733F of the plasma generator 733 acts like a single surface mirror. The beam spot of the assembly illumination beam 766B at the surface of the angled face 733F is narrow due to the low numerical aperture of the light guide 722A when immersed in the catheter fluid 132 (illustrated in FIG. 1) and will in turn form a highly concentrated spot on the balloon wall 730 and nearby tissue, such as the vessel wall 708A of the blood vessel 708. As described in greater detail herein below, this intense spot will scatter from the blood 705 and/or vessel wall 708A and be directed back toward the guide distal end 722D of the light guide 722A by the angled face 733F where it will be collected within the numerical aperture of the guide distal end 722D of the light guide 722A.

As shown in FIG. 7B, the first light energy from the portion of the assembly illumination beam 766B is generally reflected when it contacts the vessel wall 708A of the blood vessel 708 whether the balloon 704 is in a good contact condition 790A or in a bad contact condition 790B.

Returning again to FIG. 6, at step 604, at least a portion of the first light energy from the assembly illumination source is reflected and/or scattered back from the vessel wall, is guided back through the light guide, and is subsequently analyzed by the contact detector assembly.

Referring now to FIG. 7C, FIG. 7C is a simplified schematic view illustration of the embodiment of the catheter system 700 including the embodiment of the vessel wall contact detector assembly 742 illustrated in FIG. 7A, with step 604 within the operational mode being illustrated.

In particular, as illustrated, a portion of the first light energy from the assembly illumination source 766 that is incident on the balloon wall 730 is reflected and/or scattered back from the vessel wall 708A as second light energy and/or a returning energy beam 766R that is received by the distal light receiver 722R that couples the second light energy and/or the returning energy beam 766R into the guide distal end 722D of the light guide 722A. In certain embodiments, the angled face 733F of the plasma generator 733 can collect the second light energy reflected and/or scattered back from the vessel wall 708A, and redirect the second light energy back into the guide distal end 722D of the light guide 722A. Thus, the angled face 733F of the plasma generator 733 acts like a single surface mirror and can function as at least a portion of the distal light receiver 722R.

The second light energy and/or the returning energy beam 766R is then emitted from the guide proximal end 722P from where it is directed through the first optical element 764, which collimates the returning energy beam 766R. The collimated returning energy beam 766R is then directed to the second beamsplitter 778, such as the dichroic beamsplitter.

Due to the specific wavelength of the returning energy beam 766R, at least a portion of the returning energy beam 766R is redirected by the second beamsplitter 778 and then is directed through the filter 776 before it impinges on the first beamsplitter 774. Stated in another manner, the first beamsplitter 774 is positioned in the optical path of the returning energy beam 766R reflected from the second beamsplitter 778. This creates a two-way system for probing the treatment site 706 through the light guide 722A and detection of conditions there by the second light energy returned in the form of the returning energy beam 766R. The two paths provide one to transmit the assembly illumination beam 766B (illustrated in FIG. 7B) into the guide proximal end 722P of the light guide 722A and one to collect the second light energy of the returning energy beam 766R returning from the guide distal end 722D.

At least a portion of the returning energy beam 766R is then redirected by the first beamsplitter 774 through the second optical element 780 and onto the photodetector 782. The second optical element 780 focuses the collimated returning energy beam 766R, forming an image of the end face of the light guide 722A onto the photodetector 782, thereby coupling light emitted from the guide proximal end 722P of the light guide 722A. In certain non-exclusive alternative embodiments, the photodetector 782 can be a photodiode, an area sensor such as a CCD or CMOS camera, or a spectrophotometer. In suitable arrangements the chain of optical elements 764, 780 can create a high-resolution image of the end face of the light guide 722A on an image sensor.

The photodetector 782 then generates a signal based on the strength of the light at the specific wavelength. In some embodiments, the photodetector 782 generates a signal that is based on the second light energy or returning energy beam 766R reflected and/or scattered back from the vessel wall 708A that has been received by the distal light receiver 722R at or near the guide distal end 722D of the light guide 722A and that has been collected by the photodetector 782.

The signal from the photodetector 782 is directed toward the amplifier 784 where the signal from the photodetector 782 is amplified before being sent to the control electronics 786 for processing and analysis. Alternatively, in other embodiments, the contact detector assembly 742 can be designed without the amplifier 784. In such alternative embodiments, the signal from the photodetector 782 can be sent to the control electronics 786 for processing and analysis. In either embodiment, it can be stated that the signal from the photodetector 782 is being used by the control electronics 786 to determine the contact condition between the balloon wall 730 and the vessel wall 708A.

Returning again to FIG. 6, at step 608, if the analysis undertaken in step 604 determines that bad, or suboptimal, contact exists between the balloon wall and the vessel wall, then the present utilization of the balloon is modified. In certain non-exclusive situations, the modification of usage of the balloon can entail inflating the balloon to a different level (adjusting the internal balloon pressure), moving the balloon to a modified position relative to the treatment site, and/or removing the catheter and utilizing a catheter having a balloon that is more properly sized for the present treatment procedure.

At step 609, if the analysis undertaken in step 604 determines that good, or optimal, contact exists between the balloon wall and the vessel wall, then the light source (or other suitable energy source) is enabled and used within the catheter system for the desired therapeutic procedure.

Referring now to FIG. 7D, FIG. 7D is a simplified schematic view illustration of the portion of the embodiment of the catheter system 700 including the embodiment of the vessel wall contact detector assembly 742 illustrated in FIG. 7A, with step 609 within the operational mode being illustrated.

In particular, as illustrated, the light source 724 has been enabled such that the safety interlock system 762, including the interlock 762A and the shutter 762B, is no longer inhibiting the generation and/or directing of light energy from the light source 724. The pulse generator 760 is configured to trigger the light source 724 such that a pulse of light energy is generated by the light source 724. The light energy from the light source 724 is directed in the form of a source beam 724A toward the second beamsplitter 778, such as the dichroic beamsplitter in one embodiment. Stated in another manner, the second beamsplitter 778 is positioned in the optical path of the source beam 724A between the light source 724 and the guide proximal end 722P of the light guide 722A.

In certain embodiments, the second beamsplitter 778 is configured to pass or transmit light with wavelengths longer than those visible to the photodetector 782 so the source beam 724A is directed toward the guide proximal end 722P of the light guide 722A. Such threshold wavelength can be referred to as the cutoff wavelength. The second beamsplitter 778 is further configured to reflect all light having a wavelength that is shorter than the cutoff wavelength.

As illustrated in this embodiment, the first optical element 764 is positioned between the second beamsplitter 778 and the light guide 722A and is configured to collimate the source beam 724A and focus the source beam 724A down onto the guide proximal end 722P of the light guide 722A, thereby coupling the source beam 724A into the light guide 722A.

In various implementations, the source beam 724A is of such a wavelength, such as within the infrared spectrum, that it is transmitted by the second beamsplitter 778, before passing through the first optical element 764 that focuses the light energy of the source beam 724A onto the guide proximal end 722P of the light guide 722A. The light energy of the source beam 724A is then guided along and/or through the light guide 722A and emitted at the guide distal end 722D into the balloon interior 146 (illustrated in FIG. 1) of the balloon 704. The light energy of the source beam 724A impinges upon and energizes the angled face 733F of the plasma generator 733 and is redirected such that a localized plasma is generated within the catheter fluid 132 (illustrated in FIG. 1) in the balloon interior 146.

In one embodiment, the light guide 722A is a small diameter, multimode optical fiber that is used to direct light transmitted from the light source 724 and/or the assembly illumination source 766, and the plasma generator 733 contains a beveled backstop that directs light toward the vessel wall 708A. The light guide 722A and beveled backstop (angled face 733F) of the plasma generator 733 serve the dual purpose of delivering high-energy light for plasma generation (as illustrated in FIG. 7D) as well as lower energy light for blood and tissue identification (as illustrated in FIG. 7B). The beveled backstop (angled face 733F) of the plasma generator 733 is also used in conjunction with and/or as part of the distal light receiver 722R to collect light reflected and/or scattered back from illuminated regions and couple it back into the light guide 722A for transmission back to the contact detector assembly 742 for detection and analysis (as illustrated in FIG. 7C).

In summary, the embodiments of the contact detector assembly presented above all use the light guide and backstop to illuminate the space between the balloon wall and the vessel wall and collect light from this space for detection and analysis. This can be a preferred method for implementing the contact detector assembly in the existing hardware design since: 1) it uses the existing configuration of the catheter and would not increase the dimensions or crossing profile, 2) can be folded into the existing multiplexer, connector, and laser design, and 3) provides a detector for each emitter (or series of emitters at a single longitudinal position relative to the balloon length of the balloon). These are all important factors for successful implementation. The first is important because the dimensions of the catheter are critical to its maneuverability and lesion crossing performance. The third is more important to its effectiveness in detecting the layer of blood that would indicate a gap or poor contact. The layer of blood will be nonuniform around an annular region. If only one detector is present on one side of the balloon, then it would be possible for that side to be pressed into close contact while the opposing side has a large gap. The balloon could be undersized for the vessel, but the detector would indicate to the user that it was acceptable. This is also true along the full balloon length of the balloon. Having one detector on one side of the balloon could lead to many false positive indications.

Notwithstanding these advantages, one possible variation that could work around the embodiments presented would be to use a separate illumination beam guide to bring the assembly illumination beam(s) to the balloon wall and the vessel wall. One embodiment of this is shown in FIG. 8. It would have a mirror mounted at the guide distal end to direct the assembly illumination beam(s) from the assembly illumination source(s) toward the vessel wall and, in turn, collect light scattered back for detection. Examples of this are a microprism or a bevel on the endface of the illumination beam guide itself that create a side-emitting fiber through total internal reflection (TIR). This optical assembly would be run through the catheter shaft with the distal tip mounted along the lumen inside the balloon. A plurality of these could be placed in this manner along the balloon length of the balloon. However, each one would require a separate ferrule location in a connector to interface it to the system. That would be difficult with the existing multiplexer system and single-use connector with a fixed number of ferrules. However, it could be implemented as a second row of ferrules or additional ferrules interspersed with the initial ferrules for high-energy fibers.

In this alternative approach, the coupling optics and the entire contact detector assembly could be fixed relative to the illumination beam guide so that it would not have to require the multiplexer for channel switching. This could be a fixed system separate from the high-energy multiplexing system. That is outlined in FIG. 8. A plurality of fibers could be connected to this system by fusing proximal end faces together to form a single endcap for this subsystem to couple assembly illumination sources into. Without any loss of generality, it would be possible to implement this as a separate system that couples into one or a plurality of fibers that run separately from the high-energy plasma forming fibers. This could be implemented with a separate optical connector or integrated into the high-energy connector.

It is further appreciated that this configuration for the contact detector assembly can be adapted to detect whether optimal contact exists between the balloon wall and the vessel wall in any type of catheter system and catheter into which the contact detector assembly is incorporated.

FIG. 8 is a simplified schematic view illustration of still another embodiment of the catheter system 800 including still another embodiment of the vessel wall contact detector assembly 842. The design of the catheter system 800 is substantially similar to the embodiments illustrated and described herein above. It is appreciated that various components of the catheter system 800, such as are shown in FIG. 1, are not illustrated in FIG. 8 for purposes of clarity and ease of illustration. However, it is appreciated that the catheter system 800 will likely include most, if not all, of such components.

As shown in FIG. 8, the catheter system 800 again includes one or more of a light source 824 (such as a pulsed infrared laser source in one non-exclusive embodiment, or another suitable energy source), a pulse generator 860 that is coupled to the light source 824, a safety shutdown system 862 including a safety interlock 862A and a shutter 862B, a first optical element 864 (such as a coupling lens in one non-exclusive embodiment), a light guide 822A (such as an optical fiber in one non-exclusive embodiment, or another suitable energy guide), a plasma generator 833, a balloon 804 including a balloon wall 830, and the contact detector assembly 842.

As further shown in FIG. 8, the contact detector assembly 842 can include one or more of a first assembly illumination source 866, a first source driver 866A, a second assembly illumination source 868, a second source driver 868A, a first redirector 870, a second redirector 872, a beamsplitter 874, a filter 876, a third redirector 894, a second optical element 896 (such as an coupling lens in one non-exclusive embodiment), a third optical element 880 (such as an imaging lens in one non-exclusive embodiment), a photodetector 882, an amplifier 884, and control electronics 886, which can include one or more processors or circuits. It is appreciated that the contact detector assembly 842 can operate in a manner substantially similar to the first operational mode (as illustrated and described in relation to FIGS. 2 and 3A-3F) and/or the second operational mode (as illustrated and described in relation to FIGS. 4 and 5A-5C). Alternatively, the contact detector assembly 842 can be designed with only a single assembly illumination source that operates in a manner substantially similar to the operational mode illustrated and described in relation to FIGS. 6 and 7A-7D.

However, in this embodiment, the assembly illumination beam(s) 866B, 868B from the assembly illumination sources 866, 868 are directed through a separate illumination beam guide 892 rather than through the light guide 822A that is used for the source beams from the light source 824. In this embodiment, the second beamsplitter has been replaced by the third redirector 894 for simplicity, because the source beam 824A is not directed through the third redirector 894, and, thus, there is no reason to utilize a dichroic beamsplitter that reflects light at certain wavelengths and transmits light at other wavelengths.

In particular, as shown, the assembly illumination beams 866B, 868B are initially directed toward the first redirector 870 and/or the second redirector 872 before being directed toward and impinging upon the first beamsplitter 874. At least a portion of the assembly illumination beams 866B, 868B is transmitted by the first beamsplitter 874, and then passes through the filter 876 before being directed toward and impinging upon the third redirector 894.

The remaining portion of the assembly illumination beams 866B, 868B is redirected by the third redirector 894, such as a mirror, before passing through the second optical element 896 which collimates the assembly illumination beams 866B, 868B and focuses the assembly illumination beams 866B, 868B onto a guide proximal end 892P of the illumination beam guide 892. The assembly illumination beams 866B, 868B are guided through the illumination beam guide 892 and are emitted at a guide distal end 892D of the illumination beam guide 892.

The assembly illumination beams 866B, 868B are then redirected by an energy directing element 898, such as a mirror, that could be affixed to the guide distal end 892D of the illumination beam guide 892 to direct the assembly illumination beams 866B, 868B toward the balloon wall 830 and/or the vessel wall 808A. The energy directing element 898 could be further configured to redirect returning energy beams 866R, 868R that have been reflected and/or scattered back from the blood 805 and/or the vessel wall 808A back into and through the illumination beam guide 892 for subsequent analysis by the contact detector assembly 842. Alternatively, an endface at the guide distal end 892D of the illumination beam guide 892 could also be beveled to create a side-emitting feature that directs light energy out of the side and similarly collects light scattered into its numerical aperture.

The returning energy beams 866R, 868R emitted from the guide proximal end 892P of the illumination beam guide 892 can then be directed through the second optical element 896, be redirected by the third redirector 894, and be subsequently redirected by the first beamsplitter 874. The remaining portion of the returning energy beams 866R, 868R are then directed through the third optical element 880 and onto the photodetector 882. The photodetector 882 then generates a signal based on the strength of the light of each of the returning energy beams 866R, 868R at their specific wavelength. The signal from the photodetector 882 is directed toward the amplifier 884 where the signal from the photodetector 882 is amplified before being sent to the control electronics 886 for processing and analysis.

Once good contact has been determined between the balloon wall 830 and the vessel wall 808A, the light source 824 is enabled such that the safety interlock system 862, including the interlock 862A and the shutter 862B, is no longer inhibiting the generation and/or directing of light energy from the light source 824. The pulse generator 860 is configured to trigger the light source 824 such that a pulse of light energy is generated by the light source 824. The light energy from the light source 824 is directed in the form of a source beam 824A toward the first optical element 864, which is configured to collimate the source beam 824A and focus the source beam 824A down onto the guide proximal end 822P of the light guide 822A, thereby coupling the source beam 824A into the light guide 822A.

The light energy of the source beam 824A is then guided along and/or through the light guide 822A and emitted at the guide distal end 822D into the balloon interior 146 (illustrated in FIG. 1) of the balloon 804. The light energy of the source beam 824A impinges upon and energizes the angled face 833F of the plasma generator 833 and is redirected such that a localized plasma is generated within the catheter fluid 132 (illustrated in FIG. 1) in the balloon interior 146.

Thus, as noted above, in various embodiments, the contact detector assembly of the present invention is uniquely configured to: 1) detect when the balloon wall is in optimal contact with the vessel wall at the treatment site to optimize energy transmission and treatment efficacy, 2) identify when the balloon is undersized for the vessel under treatment and providing feedback to the user, and 3) detect any condition where balloon contact is suboptimal for treatment.

However, alternative approaches can also be utilized in addition to the various embodiments that have been described in detail herein above. For example, in more advanced approaches, a plurality of wavelengths would be used for a spectroscopic approach. The assembly illumination source could be broad spectrum such as a high-intensity white LED. The photodetector would then be a spectrometer. An example of this would be a grating to disperse the signal combined with a linear CCD array. Other spectral separation methods could be used such as fixed filter arrays or linear variable filters. In this approach, the returned light could be analyzed to extract spectral signatures for blood and tissue, and these spectral signatures could then be used to determine the relative amounts of each of the blood and tissue in the beam path through the space.

Eigen decomposition methods like Principal Component Analysis (PCA) or template correlation could be used for this purpose, though other methods could work.

Other factors should also be considered when designing an appropriate catheter system and/or contact detector assembly that incorporates features of the present invention. Such additional factors include, but are not limited to:
1) The treatment site is most commonly an artery, so the catheter would only be exposed to arterial blood rather than veinous blood. Arterial blood is oxygenated so the primary species will be O₂Hb. Therefore, the photodetector could work using wavelengths that are not at isosbestic points. In that case, a much simpler illumination source could be used such as a RGB diode trio. For example, the TCW-RGBS-400R is a fully integrated, three laser diode module with wavelengths at 450, 520, and 638 nm. Stated in another manner, in such implementations, an integrated multi-wavelength illumination source would typically only require a single illumination assembly for the contact detector assembly.
2) A small laser diode module like this one could be integrated right into the multiplexer optical platen. This could be merged directly into the plasma flash detector module. The photodetector in the plasma flash detector could then serve a dual purpose and become the photodetector for the vessel wall contact detector assembly. That would be a meaningful simplification and size reduction enabling a practical implementation.
3) Another embodiment that could also be a workaround solution is Optical Coherence Tomography (OCT). This can be implemented on a single optical fiber. However, it would be very difficult to do with the present catheter and multiplexer design.

FIG. 9 is a simplified schematic view illustration of an embodiment of the diameter correlation system 947 having features of the present invention, the diameter correlation system 947 incorporating input from and/or output for the pressure sensor assembly 941, the balloon compliance chart 955, the balloon diameter determination system 957, the contact detector assembly 942, the vessel diameter determination system 958, and the vessel diameter mapping system 959. In various embodiments, the diameter correlation system 947 is incorporated, at least in part, within the system controller 126 (illustrated in FIG. 1), and is uniquely configured to ensure that there is a proper correlation of the outer diameter 104BD (illustrated in FIG. 1) of the balloon 104 (illustrated in FIG. 1) and the inner diameter 108D (illustrated in FIG. 1) of the blood vessel 108 (illustrated in FIG. 1) for most effective lithotripsy therapy and/or drug delivery at the treatment site 106 (illustrated in FIG. 1).

As illustrated, the use and operation of the diameter correlation system 947 entails a multi-step process by which the diameter correlation system 947 is able to effectively ensure that there is a proper correlation of the outer diameter 104BD of the balloon 104 and the inner diameter 108D of the blood vessel 108 for most effective lithotripsy therapy and/or drug delivery at the treatment site 106.

For example, within the balloon diameter determination system 957, the pressure sensor assembly 941 can be utilized to sense and/or monitor an internal balloon pressure within the balloon interior 146 (illustrated in FIG. 1) of the balloon 104. The pressure sensor assembly 941 can thus provide data and/or information to the system controller 126 regarding the internal balloon pressure. The system controller 126 can then utilize that data and/or information regarding the internal balloon pressure in conjunction with the balloon compliance chart 955, which is configured to plot the outer diameter 104BD of the balloon 104 versus the internal balloon pressure for various different balloons in terms of style, size, design, material, etc. Thus, in such manner, the balloon diameter determination system 957 is able to determine the outer diameter 104BD of the balloon 104 as the catheter system 100 (illustrated in FIG. 1) is being utilized for performing treatment procedures at the treatment site 106. In certain embodiments, the outer diameter 104BD of the balloon 104 versus internal balloon pressure can be displayed on the GUI 127 (illustrated in FIG. 1) for use by the operator.

Subsequently, the vessel diameter determination system 958 can be utilized to determine the inner diameter 108D of the blood vessel 108 within which the catheter system 100 is being used. In particular, the contact detector assembly 942 can be utilized, in a manner such as described above, to determine when good contact exists between the balloon wall 130 (illustrated in FIG. 1) of the balloon 104 and the vessel wall 108A of the blood vessel 108. The data and/or information regarding the outer diameter 104BD of the balloon 104, as determined through use of the balloon diameter determination system 957, can then be used in conjunction with the data and/or information from the contact detector assembly 942 with regard to when good contact exists between the balloon wall 130 and the vessel wall 108A, to determine the inner diameter 108D of the blood vessel 108. Stated in another manner, when good contact exists between the balloon wall 130 of the balloon 104 and the vessel wall 108A of the blood vessel 108, then the inner diameter 108D of the blood vessel 108 is equal to the outer diameter 104BD of the balloon 104. Thus, the vessel diameter determination system 958 is able to determine the inner diameter 108D of the blood vessel 108 at the particular location within the blood vessel 108 that the catheter system is presently being used.

The vessel diameter mapping system 959 can then be utilized to effectively map the inner diameter 108D of the blood vessel 108 at various locations along the site length 106L (illustrated in FIG. 1) of the treatment site 106. More particularly, the vessel diameter determination system 958 can be utilized to determine the inner diameter 108D of the blood vessel 108, in a manner such as described above, in a plurality of locations along the site length 106L of the treatment site 106. In many embodiments, the determination of the inner diameter 108D of the blood vessel 108 can occur at evenly spaced apart locations along the site length 106L of the treatment site 106, such as every 0.1 mm, 0.2 mm, 0.5 mm, 0.8 mm, 1.0 mm, 1.5 mm, 2.0 mm, 2.5 mm, 3.0 mm, 3.5 mm, 4.0 mm, 4.5 mm, 5.0 mm, 5.5 mm, 6.0 mm, 6.5 mm, 7.0 mm, 7.5 mm, 8.0 mm, 8.5 mm, 9.0 mm, 9.5 mm, or 10.0 mm. Alternatively, the spacing between determinations of the inner diameter 108D of the blood vessel 108 along the site length 106L of the treatment site 106 can be different than those specifically noted above. Thus, with the data and/or information from the vessel diameter determination system 958 at a plurality of locations along the site length 106L of the treatment site 106, the vessel diameter mapping system 959 can effectively map out the blood vessel 108 along the full site length 106L of the treatment site 106. In certain embodiments, an image of the mapping of the inner diameter 108D of the blood vessel 108 along the site length 106L of the treatment site 106 can be displayed on the GUI 127 for use by the operator.

Finally, the diameter correlation system 947 can be utilized to ensure a proper correlation of the outer diameter 104BD of the balloon 104 and the inner diameter 108D of the blood vessel 108 for most effective lithotripsy therapy and/or drug delivery at the treatment site 106. In particular, the diameter correlation system 947 can utilize data and/or information from the vessel diameter mapping system 959 with regard to the inner diameter 108D of the blood vessel 108 along the site length 106L of the treatment site 106, in conjunction with data and/or information from the balloon diameter determination system 957 with regard to the outer diameter 104BD of the balloon 104 versus internal balloon pressure, for purposes of ensuring the proper correlation of the outer diameter 104BD of the balloon 104 and the inner diameter 108D of the blood vessel 108 for most effective lithotripsy therapy and/or drug delivery at the treatment site 106. More specifically, the diameter correlation system 947 and/or the system controller 126 can ensure that the balloon 104 is properly inflated (pressurized) so that the outer diameter 104BD of the balloon 104 effectively correlates with the inner diameter 108D of the blood vessel 108 at the particular location of the balloon 104 along the site length 106L of the treatment site 106 during lithotripsy therapy and/or drug delivery at the treatment site 106.

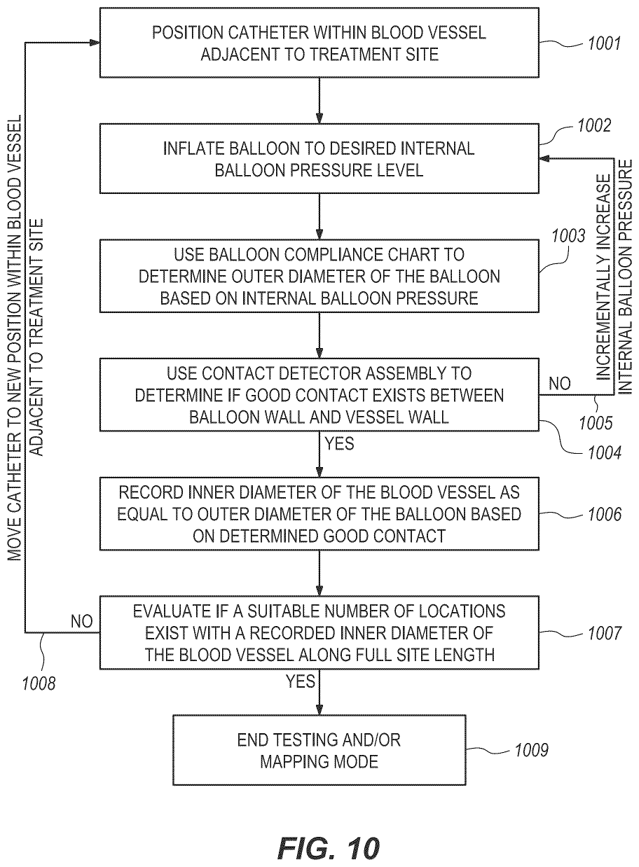

FIG. 10 is a flowchart illustrating a first (testing and/or mapping) mode of operation for the diameter correlation system of FIG. 9. It is appreciated that any of the steps listed in the flowchart of FIG. 10 can be modified, deleted and/or combined in any suitable manner, and/or the order of steps can be changed, without deviating from the intended breadth and scope of the present invention. One or more steps can also be added to the series of steps specifically illustrated and described within the flowchart without deviating from the intended breadth and scope of the present invention.

At step 1001, the user or operator positions a balloon catheter within the blood vessel to be treated adjacent to a treatment site.

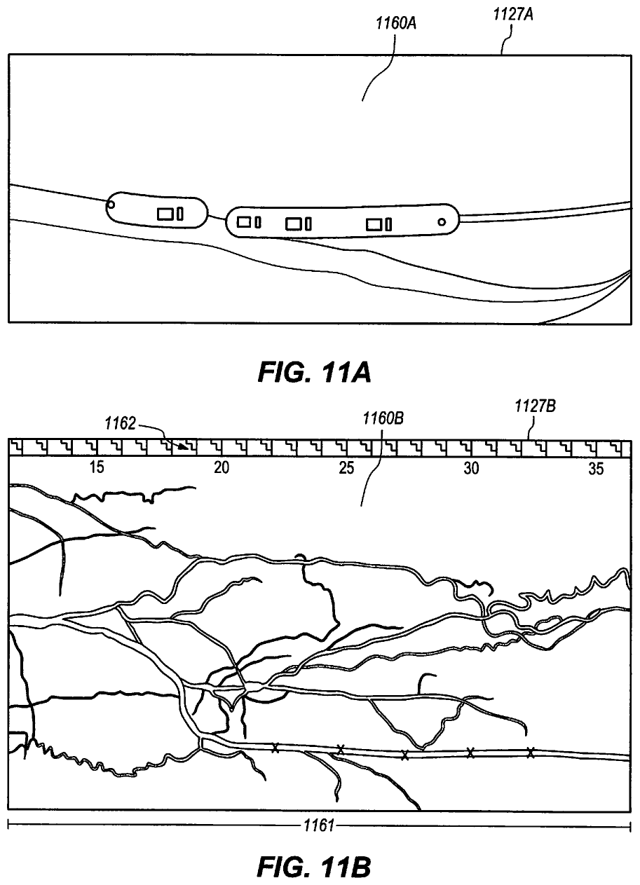

FIG. 11A illustrates a portion of one embodiment of a GUI 1127A. GUI 1127A illustrates an image 1160A showing positioning of the catheter 102 (illustrated in FIG. 1), as well as a method for ensuring proper positioning of the catheter 102, the balloon 104 (illustrated in FIG. 1) and/or the emitters 135 (illustrated in FIG. 1) of the catheter system 100 (illustrated in FIG. 1) relative to the treatment site 106 (illustrated in FIG. 1). As used herein the term “image” can include a still image or a video image, as non-exclusive examples. During angiograms using fluoroscopy, the fluoroscopy system records images and/or videos of the blood vessel 108 (illustrated in FIG. 1). Images 1160A such as shown in FIG. 11A can be recorded and/or saved for the physician or operator to reference at a later point in time during or after a procedure. The system console 123 (illustrated in FIG. 1) and/or the GUI 1127A can save one or more images, such as image files (not shown), through use of the system controller 123, and the image 1160A can subsequently be used to annotate a vessel diameter onto it as described herein. In certain embodiments, the system controller 123 can utilize image recognition software to determine location(s) of radiopaque material that can be included within the catheter 102, such as within the balloon 104, incorporated into the emitters 135, provided via optical sensors, etc., and the image 1160A can be subsequently annotated showing the inner diameter of the blood vessel 108 at the particular location along the site length of the treatment site 106.

Returning now to FIG. 10, at step 1002, the balloon is inflated to a desired internal balloon pressure level. It is appreciated that an initial internal balloon pressure level should be somewhat conservative to ensure that the outer diameter of the balloon in the inflated state is not larger than the inner diameter of the blood vessel so as to overstretch the blood vessel. For example, recognizing the blood vessels in different areas of the body can have inner diameters that range from two mm to four mm, or two mm to eight mm, the initial internal balloon pressure level can be such for the particular balloon being used that the outer diameter of the balloon will not be greater than approximately two mm. In some embodiments, the diameter correlation system can include a warning with regard to the dangers from potential overstretching of the blood vessel to better ensure that the initial internal balloon pressure is not too high and the initial outer diameter of the balloon not too large.

At step 1003, the system controller and/or the diameter correlation system utilizes the balloon compliance chart (for the appropriate balloon be used) to determine the outer diameter of the balloon based on the internal balloon pressure.

At step 1004, the contact detector assembly is utilized to determine if good contact exists between the balloon wall and the vessel wall at the particular internal balloon pressure being applied.

At (optional) step 1005, if the contact detector assembly determines that good contact does not exist between the balloon wall and the vessel wall at the particular internal balloon pressure being applied, then the internal balloon pressure should be incrementally increased, such that the outer diameter of the balloon would be incrementally increased in an appropriate manner toward a status of good contact between the balloon wall and the vessel wall, while still ensuring that the balloon does not overstretch the blood vessel. For example, in certain non-exclusive implementations, the incremental increase in the internal balloon pressure can be such that the outer diameter of the balloon increases by no more than 0.1 mm, 0.2 mm, 0.3 mm, 0.4 mm, 0.5 mm, 0.6 mm, 0.7 mm, 0.8 mm, 0.9 mm, or 1.0 mm from one step to the next. The incremental increase of the internal balloon pressure within the balloon effectively returns the procedure to step 1002, from where the procedure continues in a similar manner as noted above.

At step 1006, if the contact detector assembly determines that good contact does exist between the balloon wall and the vessel wall at the particular internal balloon pressure being applied, then the inner diameter of the blood vessel is recorded to being equal to the outer diameter of the balloon at that particular location along the site length of the treatment site.

FIG. 11B illustrates a portion of one embodiment of a GUI 1127B. GUI 1127B illustrates an image 1160B showing a full site length 1161 (spanning the width of FIG. 11B) of the treatment site 106 (illustrated in FIG. 1) with markings (illustrated as “X” 's) at specified periodic locations along the site length 1161 which can show an inner diameter of a blood vessel 108 (illustrated in FIG. 1) at such locations as part of the vessel diameter mapping system. As shown in FIG. 11B, in certain embodiments, a radiopaque ruler 1162 can be placed on the patient, which can be used to take reference measurements of some or all of the site length 1161 of the treatment site 106 within the blood vessel 108. The image 1160B, as shown on the GUI 1127B, can also allow for multiple annotations regarding the inner diameter of the blood vessel 108 along the full site length 1161 of the treatment site 106, thus providing an accurate measurement of the inner diameter of the blood vessel 108 along the full site length 1161 of the treatment site 106 to be treated. It is appreciated that this data can be later used to increase the likelihood that an appropriate balloon size is employed during any lithotripsy procedure and/or drug therapy employed at various locations along the full site length 1161 of the treatment site 106.

Returning again to FIG. 10, at step 1007, the diameter correlation system evaluates whether or not a suitable number of locations exist with a recorded inner diameter of the blood vessel along the full site length of the treatment site within the blood vessel. It is appreciated that the number of locations required for purposes of determining whether or not a suitable number of locations exist with a recorded inner diameter of the blood vessel will depend, at least in part, on the full site length of the treatment site.

At (optional) step 1008, if the diameter correlation system determines that a suitable number of locations have not been recorded with the inner diameter of the blood vessel along the full length of the treatment site, then the catheter is moved to a new position within the blood vessel adjacent to the treatment site. In particular, the catheter can be moved an incremental distance along the site length of the treatment site, such as by approximately, 0.1 mm, 0.2 mm, 0.5 mm, 0.8 mm, 1.0 mm, 1.5 mm, 2.0 mm, 2.5 mm, 3.0 mm, 3.5 mm, 4.0 mm, 4.5 mm, 5.0 mm, 5.5 mm, 6.0 mm, 6.5 mm, 7.0 mm, 7.5 mm, 8.0 mm, 8.5 mm, 9.0 mm, 9.5 mm, 10.0 mm, or another suitable value. The incremental movement of the catheter along the site length of the treatment site effectively returns the procedure to step 1001, from where the procedure continues in a similar manner as noted above.

At step 1009, if the diameter correlation system determines that a suitable number of locations have been recorded with the inner diameter of the blood vessel along the full length of the treatment site, then the testing and/or mapping mode of operation for the diameter correlation system is ended.

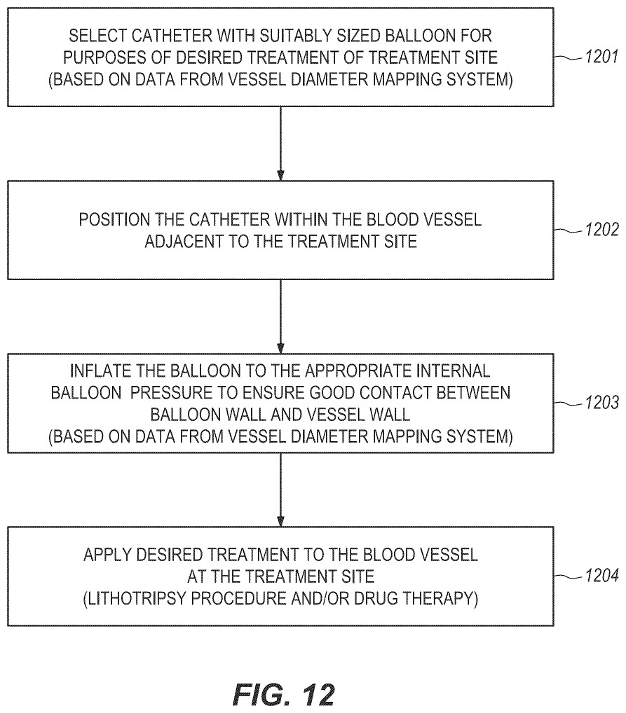

FIG. 12 is a flowchart illustrating a second (therapeutic use) mode of operation for the diameter correlation system of FIG. 9. It is appreciated that any of the steps listed in the flowchart of FIG. 12 can be modified, deleted and/or combined in any suitable manner, and/or the order of steps can be changed, without deviating from the intended breadth and scope of the present invention. One or more steps can also be added to the series of steps specifically illustrated and described within the flowchart without deviating from the intended breadth and scope of the present invention.

At step 1201, the physician or operator selects a catheter with a suitably sized balloon for purposes of a desired treatment of the treatment site. As described, the suitably sized balloon will be selected based on data as obtained through use of the vessel diameter mapping system. It is appreciated that it is desired to select a catheter with a suitably sized balloon for both any desired lithotripsy procedure and any desired blood therapy treatment procedure for the various locations along the full site length of the treatment site.

At step 1202, the catheter, with the suitably sized balloon incorporated therein, is positioned within the blood vessel adjacent to the treatment site.

At step 1203, the balloon is inflated to the appropriate internal balloon pressure to ensure good contact between the balloon wall and the vessel wall. Stated in another manner, the balloon is inflated to the appropriate internal balloon pressure to ensure that the outer diameter of the balloon (based on data and/or information for the balloon diameter determination system) effectively correlates with the inner diameter of the blood vessel (based on data and/or information from the vessel diameter mapping system) at the particular location of the balloon along the full site length of the treatment site.

At step 1204, the physician or operator applies the desired treatment to the blood vessel at the treatment site. It is appreciated that the desired treatment can include a lithotripsy procedure and/or drug therapy to be applied to the blood vessel at the treatment site.

It is appreciated that these steps are repeated as many times as necessary to ensure that the desired treatment to the blood vessel is effectively provided at all suitable and/or desired locations along the full site length of the treatment site.

It should be noted that, as used in this specification and the appended claims, the singular forms “a,” “an,” and “the” include plural referents unless the content and/or context clearly dictates otherwise. It should also be noted that the term “or” is generally employed in its sense including “and/or” unless the content or context clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

The headings used herein are provided for consistency with suggestions under 37 CFR 1.77 or otherwise to provide organizational cues. These headings shall not be viewed to limit or characterize the invention(s) set out in any claims that may issue from this disclosure. As an example, a description of a technology in the “Background” is not an admission that technology is prior art to any invention(s) in this disclosure. Neither is the “Summary” or “Abstract” to be considered as a characterization of the invention(s) set forth in issued claims.

The embodiments described herein are not intended to be exhaustive or to limit the invention to the precise forms disclosed in the detailed description provided herein. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

It is understood that although a number of different embodiments of the catheter system, the diameter correlation system and/or the contact detector assembly have been illustrated and described herein, one or more features of any one embodiment can be combined with one or more features of one or more of the other embodiments, provided that such combination satisfies the intent of the present invention.

While a number of exemplary aspects and embodiments of the catheter system, the diameter correlation system and/or the contact detector assembly have been discussed above, those of skill in the art will recognize certain modifications, permutations, additions, and sub-combinations thereof. It is therefore intended that the following appended claims and claims hereafter introduced are interpreted to include all such modifications, permutations, additions, and sub-combinations as are within their true spirit and scope, and no limitations are intended to the details of construction or design herein shown.

What is claimed is:

1. A catheter system for treating a treatment site within or adjacent to a vessel wall of a blood vessel, the catheter system comprising:
a balloon that is positionable substantially adjacent to the vessel wall at the treatment site, the balloon having a balloon wall that defines a balloon interior;
a first assembly illumination source that generates a first assembly illumination beam that moves in a first direction into the balloon interior; and
a contact detector assembly that is configured to optically analyze a first returning energy beam from the balloon interior that moves in a second direction that is opposite the first direction, the contact detector assembly being configured to analyze the first returning energy beam to determine a contact condition between the balloon wall and the vessel wall.

2. The catheter system of claim 1 wherein the balloon is configured to receive and retain a catheter fluid within the balloon interior; and wherein the catheter system further comprises a pressure sensor assembly including a pressure sensor that is configured to sense an internal balloon pressure of the catheter fluid within the balloon interior, the pressure sensor being in fluid communication with the catheter fluid retained within the balloon interior.

3. The catheter system of claim 2 further comprising a balloon compliance chart that plots an outer diameter of the balloon versus the internal balloon pressure, and a balloon diameter determination system that determines the outer diameter of the balloon based on the sensed internal balloon pressure and the balloon compliance chart.

4. The catheter system of claim 3 further comprising a vessel diameter determination system that is configured to determine an inner diameter of the blood vessel; wherein the contact detector assembly is configured to analyze the first returning energy beam to determine when good contact exists between the balloon wall and the vessel wall; and wherein when the contact detector assembly determines that good contact exists between the balloon wall and the vessel wall, the vessel diameter determination system determines that the inner diameter of the blood vessel is equal to the outer diameter of the balloon.

5. The catheter system of claim 4 wherein the treatment site has a site length; and wherein the vessel diameter determination system is configured to determine the inner diameter of the blood vessel at multiple locations along the site length of the treatment site.

6. The catheter system of claim 5 further comprising a system controller including one or more processors, and a diameter correlation system that is at least partially integrated within the system controller; and wherein the diameter correlation system is configured to utilize data from the balloon diameter determination system regarding the outer diameter of the balloon, and data from the vessel diameter determination system regarding the inner diameter of the blood vessel at the multiple locations along the site length of the treatment site, for purposes of ensuring a proper correlation of the outer diameter of the balloon and the inner diameter of the blood vessel during use of the catheter system in a therapeutic procedure.

7. The catheter system of claim 1 further comprising a beam guide; wherein the first assembly illumination beam moves in the first direction through the beam guide from a guide proximal end to a guide distal end that is positioned within the balloon interior, the first assembly illumination beam including first light energy; and wherein the first returning energy beam moves in the second direction through the beam guide from the guide distal end to the guide proximal end, the first returning energy beam including second light energy from at least a portion of the first light energy being reflected from the vessel wall.

8. The catheter system of claim 7 further comprising a second assembly illumination source that generates a second assembly illumination beam that moves in the first direction into the balloon interior; and wherein the contact detector assembly is configured to optically analyze a second returning energy beam from the balloon interior that moves in the second direction that is opposite the first direction, the contact detector assembly being configured to analyze the first returning energy beam and the second returning energy beam to determine the contact condition between the balloon wall and the vessel wall.

9. The catheter system of claim 8 wherein the second assembly illumination beam moves in the first direction through the beam guide from the guide proximal end to the guide distal end that is positioned within the balloon interior, the second assembly illumination beam including first light energy; and wherein the second returning energy beam moves in the second direction through the beam guide from the guide distal end to the guide proximal end, the second returning energy beam including second light energy from at least a portion of the first light energy from the second assembly illumination beam being reflected from blood that is positioned between the balloon wall and the vessel wall.

10. The catheter system of claim 9 wherein the first assembly illumination beam is at a first wavelength; and wherein the second assembly illumination beam is at a second wavelength that is different than the first wavelength.

11. The catheter system of claim 7 further comprising an energy source that generates a source beam that is directed into the balloon interior; wherein the source beam is directed through the beam guide from a guide proximal end to a guide distal end that is positioned within the balloon interior; wherein the balloon is configured to receive and retain a catheter fluid within the balloon interior; and wherein the source beam being directed into the balloon interior induces generation of a plasma in the catheter fluid within the balloon interior.

12. The catheter system of claim 7 further comprising an energy source that generates a source beam that is directed into the balloon interior; and an energy guide that is separate from the beam guide; wherein the source beam is directed through the energy guide from a guide proximal end to a guide distal end that is positioned within the balloon interior; wherein the balloon is configured to receive and retain a catheter fluid within the balloon interior; and wherein the source beam being directed into the balloon interior induces generation of a plasma in the catheter fluid within the balloon interior.

13. The catheter system of claim 8 wherein the contact detector assembly includes a beamsplitter and a photodetector, the beamsplitter being configured to receive returning energy that has moved through the beam guide in the second direction from the guide distal end to the guide proximal end, and direct at least a portion of the returning energy to the photodetector; and wherein the photodetector generates a signal based at least in part on the portion of the returning energy that is directed to the photodetector, the signal being used by control electronics to determine the contact condition between the balloon wall and the vessel wall.

14. A method for treating a treatment site within or adjacent to a vessel wall of a blood vessel, the method comprising the steps of:
positioning a balloon substantially adjacent to the vessel wall at the treatment site, the balloon having a balloon wall that defines a balloon interior;
generating a first assembly illumination beam with a first assembly illumination source;
moving the first assembly illumination beam in a first direction into the balloon interior;
moving a first returning energy beam from the balloon interior in a second direction that is opposite the first direction; and
optically analyzing the first returning energy beam from the balloon interior with a contact detector assembly to determine a contact condition between the balloon wall and the vessel wall.

15. The method of claim 14 further comprising the steps of receiving and retaining a catheter fluid within the balloon interior; sensing an internal balloon pressure of the catheter fluid within the balloon interior with a pressure sensor of a pressure sensor assembly; and determining an outer diameter of the balloon with a balloon diameter determination system based on the sensed internal balloon pressure and a balloon compliance chart that plots the outer diameter of the balloon versus the internal balloon pressure.

16. The method of claim 15 further comprising the step of determining an inner diameter of the blood vessel with a vessel diameter determination system; wherein the step of optically analyzing includes optically analyzing the first returning energy beam with the contact detector assembly to determine when good contact exists between the balloon wall and the vessel wall; and wherein when the contact detector assembly determines that good contact exists between the balloon wall and the vessel wall, the vessel diameter determination system determines that the inner diameter of the blood vessel is equal to the outer diameter of the balloon.

17. The method of claim 16 wherein the treatment site has a site length; wherein the step of determining the inner diameter of the blood vessel includes determining the inner diameter of the blood vessel with the vessel diameter determination system at multiple locations along the site length of the treatment site; and further comprising the step of ensuring a proper correlation of the outer diameter of the balloon and the inner diameter of the blood vessel during use of the catheter system in a therapeutic procedure with a diameter correlation system that is at least partially integrated within a system controller including one or more processors, the diameter correlation system utilizing data from the balloon diameter determination system regarding the outer diameter of the balloon, and data from the vessel diameter determination system regarding the inner diameter of the blood vessel at the multiple locations along the site length of the treatment site.

18. The method of claim 14 further comprising the steps of generating a second assembly illumination beam with a second assembly illumination source; moving the second assembly illumination beam in the first direction into the balloon interior; and moving a second returning energy beam from the balloon interior in the second direction that is opposite the first direction; and wherein the step of optically analyzing includes optically analyzing the first returning energy beam and the second returning energy beam from the balloon interior with the contact detector assembly to determine the contact condition between the balloon wall and the vessel wall.

19. The method of claim 18 wherein the step of moving the first assembly illumination beam includes moving the first assembly illumination beam in the first direction through a beam guide from a guide proximal end to a guide distal end that is positioned within the balloon interior, the first assembly illumination beam including first light energy that is at a first wavelength; wherein the step of moving the first returning energy beam includes moving the first returning energy beam in the second direction through the beam guide from the guide distal end to the guide proximal end, the first returning energy beam including second light energy from at least a portion of the first light energy being reflected from the vessel wall; wherein the step of moving the second assembly illumination beam through the beam guide from the guide proximal end to the guide distal end that is positioned within the balloon interior, the second assembly illumination beam including first light energy that is at a second wavelength that is different than the first wavelength; and wherein the step of moving the second returning energy beam includes moving the second returning energy beam through the beam guide from the guide distal end to the guide proximal end, the second returning energy beam including second light energy from at least a portion of the first light energy from the second assembly illumination beam being reflected from blood that is positioned between the balloon wall and the vessel wall.

20. A catheter system for treating a treatment site within or adjacent to a vessel wall of a blood vessel, the treatment site having a site length, the catheter system comprising:
a balloon that is positionable substantially adjacent to the vessel wall at the treatment site, the balloon having a balloon wall that defines a balloon interior, the balloon being configured to receive and retain a catheter fluid within the balloon interior;
a pressure sensor assembly including a pressure sensor that is configured to sense an internal balloon pressure of the catheter fluid within the balloon interior, the pressure sensor being in fluid communication with the catheter fluid retained within the balloon interior;
a balloon compliance chart that plots an outer diameter of the balloon versus the internal balloon pressure;
a balloon diameter determination system that determines the outer diameter of the balloon based on the sensed internal balloon pressure and the balloon compliance chart,
a beam guide including a guide proximal end and a guide distal end, the guide distal end being positioned within the balloon interior;
a first assembly illumination source that generates a first assembly illumination beam that moves through the beam guide in a first direction from the guide proximal end to the guide distal end, the first assembly illumination beam including first light energy that is at a first wavelength, the first assembly illumination beam being directed from the guide distal end toward the vessel wall;
a second assembly illumination source that generates a second assembly illumination beam that moves through the beam guide in the first direction from the guide proximal end to the guide distal end, the second assembly illumination beam including first light energy that is at a second wavelength that is different than the first wavelength, the second assembly illumination beam being directed from the guide distal end toward the vessel wall;
a contact detector assembly that is configured to optically analyze (i) a first returning energy beam from the balloon interior that moves through the beam guide in a second direction from the guide distal end to the guide proximal end, the first returning energy beam including second light energy from at least a portion of the first light energy being reflected from the vessel wall, and (ii) a second returning energy beam from the balloon interior that moves through the beam guide in the second direction from the guide distal end to the guide proximal end, the second returning energy beam including second light energy from at least a portion of the first light energy from the second assembly illumination beam being reflected from blood that is positioned between the balloon wall and the vessel wall, the contact detector assembly being configured to analyze the first returning energy beam and the second returning energy beam to determine a contact condition between the balloon wall and the vessel wall;
a vessel diameter determination system that is configured to determine an inner diameter of the blood vessel; wherein when the contact detector assembly determines that good contact exists between the balloon wall and the vessel wall, the vessel diameter determination system determines that the inner diameter of the blood vessel is equal to the outer diameter of the balloon, the vessel diameter determination system being configured to determine the inner diameter of the blood vessel at multiple locations along the site length of the treatment site;
a system controller including one or more processors; and
a diameter correlation system that is at least partially integrated within the system controller, the diameter correlation system being configured to utilize data from the balloon diameter determination system regarding the outer diameter of the balloon, and data from the vessel diameter determination system regarding the inner diameter of the blood vessel at the multiple locations along the site length of the treatment site, for purposes of ensuring a proper correlation of the outer diameter of the balloon and the inner diameter of the blood vessel during use of the catheter system in a therapeutic procedure.
